SEQUENCE LISTING (2 sequences)

SEQ ID NO: 1 - DNA, 36 bp, synthetic construct
  misc_feature 1..36: note = Anchor ssDNA - Anti-Lysozyme Aptamer
  source 1..36: mol_type = other DNA
ttttttatcagggctaaagagtgcagagttacttag

SEQ ID NO: 2 - DNA, 36 bp, synthetic construct
  misc_feature 1..36: note = Complementary analyte ssDNA
  source 1..36: mol_type = other DNA
ctaagtaactctgcactctttagccctgataaaaaa

(19) United States
(12) Patent Application Publication
(10) Pub. No.: US 2024/0402165 A1
(43) Pub. Date: Dec. 5, 2024

(54) SPIKY METAL STRUCTURES

(71) Applicant: YEDA RESEARCH AND DEVELOPMENT CO. LTD., Rehovot (IL)

(72) Inventors: Ulyana SHIMANOVICH, Rehovot (IL); Aleksei V. SOLOMONOV, Rehovot (IL); Alexander TESLER, Rehovot (IL)

(73) Assignee: YEDA RESEARCH AND DEVELOPMENT CO. LTD., Rehovot (IL)

(21) Appl. No.: 18/688,809

(22) PCT Filed: Sep. 5, 2022
(86) PCT No.: PCT/IL2022/050967

§ 371 Date: Mar. 4, 2024

(30) Foreign Application Priority Data
Sep. 5, 2021 (IL) 286155

(51) Int. Cl. G01N 33/543

(52) U.S. Cl. CPC G01N 33/54346; G01N 33/54386

(57) ABSTRACT

This invention provides nanostructures comprising a base. and nano-protrusions attached to the base. The invention further provides arrays of such nanostructures on substrates. Also provided by this invention are analysis and catalysis. separation and purification systems and methods. comprising or making use of the novel nanostructures. Particles and films comprising nano-protrusions are included in this invention as well.

### SEQUENCE LISTING STATEMENT

The instant application contains a Sequence Listing which has been submitted electronically in .xml format and is hereby incorporated by reference in its entirety. Said .XML copy, created on Aug. 31, 2022, is named P-605999-PCT_31AUG22.xml and is 3.40 Kilo bytes in size.

### FIELD OF THE INVENTION

This invention provides nanostructures comprising a base, and nano-protrusions attached to the base. The invention further provides arrays of such nanostructures on substrates. Also provided by this invention are analysis and catalysis, separation and purification systems and methods comprising or making use of the novel nanostructures. Particles and films comprising nano-protrusions are included in this invention as well.

### BACKGROUND OF THE INVENTION

Small particles that exhibit large surface area are important for many uses, among which are applications such as catalysis and detection. The small size of the particles, their large surface area and the morphology of the particles are advantageous for some applications as it allows facile binding of certain molecules to their surface, sometimes in amounts that are much higher than what would bind to bulk or to a large flat surface. Binding of molecules to the particle surface enables analysis of these molecules and also may catalyze a chemical reaction involving the bound molecules. Such binding also allows indirect reaction/analysis of compounds that interact with the molecules.

One analysis technique that utilize such binding is spectroscopy. Numerous spectroscopic techniques utilize the enhancement of signal from surfaces of substrates to which analyte molecules are bound directly or indirectly (through a linker molecule or spacer). These techniques are termed surface enhanced spectroscopies (SES). One such SES is surface enhanced Raman spectroscopy (SERS). Raman spectroscopy is a powerful and widely applied technique for material characterization, yielding detailed spectral structural fingerprints of the substances under study. One advantage of Raman spectroscopy over other vibrational spectroscopy techniques such as infrared (IR) spectroscopy is the detection of low wavenumber frequencies that correspond to e.g. metal-oxide and metal-carbon bonds which cannot be detected by IR spectroscopy. Raman spectroscopy relies on Raman scattering of photons (inelastic scattering) to yield information about the vibrational modes in a system. As typically 1 in 10⁸ a.u. of the incident radiation undergoes spontaneous Raman scattering, the detection limit of the technique is inherently low. A serendipitous discovery from 1974 has ultimately provided means to overcome this challenge. An enhancement of the pyridine spectrum measured on silver electrode was found, which could not be correlated to the concentration of the compound on the substrate. It was later postulated that excitation of localized surface plasmon or the formation of charge-transfer complexes of for example rough metal surfaces could lead to Raman signal enhancement of up to 10¹⁰/10¹¹, which in theory allows detection at the single-molecule level. The Raman signal sensitivity-enhancing technique that followed (SERS) had become a widely applied research technique in a broad range of fields. For example, in spectroelectrochemistry, sensor technologies, biomolecule detection and mechanistic elucidation in catalysis. SERS is clearly a useful technique with a wide range of applications.

Over the last few years, useful synthesis approaches for specific SES transducers have been developed and reported. Most of the transducers are based on gold or silver or their composites in different sizes, shapes, and aggregation states.

A large portion of SES techniques are based on the preparation of nanoparticles in solution, followed by exposure of these nanoparticles to the molecules of interest. The interactions between the nanoparticles and the molecules of interest enables its further detection and analysis (structural or behavioral). Efforts have been made to create SES transducers with well-defined and homogeneously distributed so-called “hot spots”, where the strongest signal enhancement occurs due to localized surface plasmon resonance (LSPR) coupling, which is strongest in these hot spots. Hot spots often exist at junctions between plasmonically-active metallic nanostructures. For example, a junction between two gold or silver nanoparticles or between a nanoparticle and a metal surface. In such junctions, gap plasmon modes can form, which can significantly enhance signal intensity in Raman, infrared, fluorescence and LSPR spectroscopies. Combination of some of these methods with atomic force microscopy (AFM) with its modification for tip-enhanced spectroscopies can be considered as well.

For example, LSPR spectroscopy is mainly based on the effect of absorbance band appearance in UV-Vis region upon interaction with resonant light wavelength. The plasmon resonant frequency (wavelength) as well as its intensity are very sensitive to refractive index of the environment and these parameters are changed when the refractive index changes. This may happen upon interaction of plasmon nanostructures with ambient solution, as well as due to interaction with an interface in which the nanostructures have been placed or due to interaction with specific molecules adsorbed on the surface of the nanostructures. These changes can be tracked by observing a shift in plasmonic resonance band in the spectrum.

One of the main parameters of these interactions is refractive index sensitivity, which is defined by the ratio of the resonance wavelength shift (or alternatively the intensity increase) to the change of refractive index of the surrounding medium. For LSPR spectroscopy and biosensors based on this phenomenon, it is extremely important to register a large distinct variation of resonance wavelength with small refractive index change of the environment. Finding and choosing the appropriate particle size and shape in order to increase the refractive index sensitivity is a very important task and challenge for modern science and technology.

Efforts to transfer this effect from solution to a surface have been made and include methods such as nanoparticles adsorption followed by drying (self-assembly of nanoparticles into loosely packed arrays), nanoimprinting, e-beam and laser interference lithography, focused ion beam milling, and template-based technologies. However, these methods are expensive and require large number of resources and in many cases appears to be time consuming and hardly scalable. Moreover, many existing methods are restricted by low level of flexibility in device manufacturing in terms of adaptation to specific customer needs.

Facile, reproducible and reusable synthesis procedures for producing SES transducers not in the liquid phase are still lacking. A common problem in the preparation protocol of plasmonic nanostructures, which is generally performed in the liquid phase, is the introduction of capping-agents. The initial liquid phase required for nanoparticles generation and surface modifications, cannot be performed in a solid phase. Attempts to form and modify particles in a solid phase have led to structural instability. In liquid phase, capping agents such as citrate/ascorbate ions or surfactants are used to stabilize nanoparticles in solution. Stabilization includes preventing particle aggregation with time, repulsing them from each other or producing specific surface charge on a nanoparticle. However, when it comes to end point application, for example analysis of an analyte of interest, the analyte's buffers which possesses high ionic strength often lead to particle aggregation, i.e., non-reproducible optical signals due to significant change in the nanoparticles closest environment. During the preparation of solid-state substrates, such interfering molecules have to be removed via additional chemical modification or using plasma cleaning. Such removal causes substrate instability. In the case where capping agents remain on the surface, they may interfere interaction with the analyte. In addition, the presence of capping agents either in solution-based nanoparticles dispersion or when being a part of solid-state substrate after deposition from liquid phase, may result in difficulties in analysis.

Therefore, solutions for overcoming these technological issues to allow simplification of the manufacturing process are the matter of intensive research. Specifically, a reproducible, facile method for forming such particles/structures on a solid substrate is needed.

### SUMMARY OF THE INVENTION

In one embodiment, this invention provides an array of gold nanostructures (e.g., nanostars) attached to a solid surface. The nanostars are produced on the surface of a solid substrate using a two-step formation process. The process involves the initial formation of metal (e.g. gold) islands on a substrate using metal (e.g. gold) vapor deposition followed by optional post-deposition treatment. The second step involves formation of metal (e.g. gold) spikes on the islands using wet deposition. The wet deposition involves reduction of metal (e.g. gold) ions from liquid solution onto the gold islands, thus growing spikes, needles (or other protruding shapes, as outlined herein) on the islands to form “(nano) stars” or “hedgehog” structures. The shape, size, geometry and distribution of the nano-protrusions is controlled by varying the experimental conditions. The combination of the nanostars shape and small size is an advantage for their function as spectroscopic transducers and as catalysts. The attachment of nanostars to solid surfaces allows them to be used repeatedly as the substrate carrying the nanostars can be easily rinsed/cleaned.

In one embodiment the initial step of gold vapor deposition results in films, and the films are subsequently subjected to high temperature exposure. According to this aspect and in one embodiment, the high temperature treatment (annealing) converts the films to isolated metal (e.g. gold) islands. In some embodiments, the high temperature treatment causes other beneficial modifications, such as enhancing binding of the islands to the substrate.

In one embodiment the initial step of metal vapor deposition already results in the formation of isolated islands. According to this aspect and in one embodiment, the islands are not further exposed to high temperature treatment. However, according to another embodiment, these islands are subsequently subjected to high temperature exposure. According to this aspect and in one embodiment, the high temperature treatment (annealing) causes one or more of the following: it shapes the islands, it changes the crystallinity of the islands, it strengthens the attachment of the islands to the substrate, it embeds the islands into the substrate.

As noted above, one of the uses of such nanostar arrays is for spectroscopic assays of analyte molecules/compounds. In this aspect and in one embodiment, the analyte molecule is attached to the nanostar, and the attachment is being monitored using optical, spectroscopic and/or other colorimetric techniques. The techniques, namely surface enhanced spectroscopies (SES) include but are not limited to: surface enhanced Raman spectroscopy (SERS), localized surface plasmon resonance (LSPR), surface-enhanced infrared absorbance spectroscopy (SEIRAS), surface-enhanced fluorescence (plasmon-enhanced) spectroscopy (SEFRS) and surface enhanced circular dichroism (SECD). The amount and intrinsic characteristics of the analyte can be quantitatively and qualitatively evaluated using such techniques. The application of the nanostar array is detailed as follows: in one embodiment, in this work, a facile, scalable 3-step preparation procedure SES transducers is presented, the procedure is based on the formation of gold island layers deposited directly on substrates followed by the growth of gold spikes using a simple wet-chemistry procedure. The 3-step process includes (1) gold evaporation of desired thickness on a substrate, (2) high temperature annealing causing formation of gold nanoparticles, embedded in the substrate (gold islands) and (3) formation of spikes or protrusions (rough surface) with desired geometry using wet-chemical approach. The transducers are comprised of two components: (i) Au nanostructures and (ii) the substrate on which the nanostructures are grown (for example borosilicate glass). Production of the transducers is possible due to mechanical stabilization of the initially-prepared Au islands by their partial embedding into the amorphous glass matrix by means of high temperature annealing. One major advantage of this approach is the ability to exclude other substances (e.g. surfactants or capping ligands) or impurities, that may affect processes under study with the transducers and plasmonic shift caused by particle movement. In addition, the formed nanostructures allow to work with solutions having high ionic strength without worry of destroying a sample or causing particles aggregation, what may happen in solutions. For example, processes such as self-assembly of analyte molecules (e.g. proteins) on the transducer surface are enabled by the novel transducers. Furthermore, patterns can be made using this production procedure which can be useful for different applications. For example, the formation of wells or spots, and the integration with microfluidic devices. Surface patterning can also be combined with the preparation of the novel nanostructures. Application of shadow masks for patterning allows selective deposition of gold on specific areas of a substrate during the gold evaporation process (step i) followed by surface modification, namely spikes formation. This allows case and facile preparation of different patterns for many technological purposes. For instance, patterning of spots of various sizes provides formation of an ordered array of structures for ELISA-like substrates for multiple parallel and potentially automated analysis; patterning allows integration of gold nanostar-based substrates with other technological solutions like PDMS-based structures, usually used for microfluidic device manufacturing. The method described above can be extended to include the formation of nanostars made of other metals and alloys.

In one embodiment, in order to provide high flexibility of spiked gold-structure production and device production and to fit to specific purposes and due to the relatively high cost of stainless steel-made shadow masks available in the market, a low cost, facile and fast method of shadow masks production has been herein proposed.

As noted herein above, some advantages of the novel gold structures preparation method are: (i) a facile 3-step technique (2-step+1-chemical), (ii) surface free of capping agents, (iii) stable adhesion of Au to the substrate, allowing patterning modifications, (iv) devices are stable and reusable, (v) devices are stable in wide range of pH and are not sensitive to high ionic strength. In one embodiment, the 3-step technique includes 2-steps of (i) forming a film and (ii) treat the film to form islands, and a third step (iii) which comprises production of the spikes or nano-protrusions on the islands. Production of the spikes or nano-protrusions in the third step is conducted from a liquid comprising metal ions (e.g. gold ions). This is a wet chemical step. In one embodiment, the preparation process described herein is not limited to the formation of protrusions on noble-metal islands, but is also extended to the formation of nano-protrusions on continuous or partially-continuous noble-metal films and on holes comprising a noble metal onto which the nano-protrusions are grown.

In other embodiments, a procedure of the invention is viewed as a 2-step method wherein the first step comprises forming gold islands on a substrate and the second step comprises forming the spikes on the islands. According to this aspect and in one embodiment, a plurality of methods are utilized to form the islands on the substrate. Such methods will be detailed herein below.

In one embodiment, an array of nanostars or nano-protrusions is produced on a surface of a substrate. In one embodiment, the nanostar or nano-protrusion array is used for catalysis. According to this aspect and in one embodiment, the small size, high surface area, the special geometry or any combination thereof contribute to enhanced catalytic activity as will be further described below.

According to this aspect and in one embodiment, the array is used for photocatalysis. In one embodiment, nanostructures of this invention are formed on tin oxide or on indium tin oxide. In one embodiment, nanostructures of this invention are formed on TiO₂.

In one embodiment, this invention provides a nanostructure comprising:
a metallic base;
metallic nano-protrusions comprising a first end and a second end;
   wherein, said first end is attached to said base and said second end is exposed to the environment; and
   wherein said metallic base and said metallic nano-protrusions comprise a noble metal or an alloy comprising a noble metal.

In one embodiment, this invention provides a nanostructure comprising:
an isolated metallic base;
metallic nano-protrusions comprising a first end and a second end;
   wherein, said first end is attached to said base and said second end is exposed to the environment; and
   wherein said metallic base and said metallic nano-protrusions comprise a noble metal or an alloy comprising a noble metal.

In one embodiment, the shape of said nano-protrusions is selected from spikes, rounded structures, rods, balls, domes, squares, rectangles, oval, irregular-shaped or any combination thereof.

In one embodiment, this invention provides a nanostructure comprising:
a metallic base;
metallic nanospikes comprising a first end and a second end;
   wherein, said first end is attached to said base and said second end is exposed to the environment; and wherein said metallic base and said metallic nanospikes comprise a noble metal or an alloy comprising a noble metal.

In one embodiment, this invention provides a nanostructure comprising:
an isolated metallic base;
metallic nanospikes or nano-protrusions comprising a first end and a second end;
   wherein, said first end is attached to said base and said second end is exposed to the environment; and wherein said metallic base and said metallic nanospikes comprise a noble metal or an alloy comprising a noble metal.

In one embodiment, the length of at least one dimension of said nanostructure ranges between 1 nm and 2 mm. In one embodiment, the length of at least one dimension of said nanostructure ranges between 1 nm and 100 μm. In one embodiment, the base comprises a flattened shape, wherein the length of at least one lateral dimension of said shape is larger than the height of said shape. In one embodiment, the base comprises a non-flattened shape, wherein the length of at least one lateral dimension of said shape is smaller or equivalent to the height of said shape.

In one embodiment, the length of at least one lateral dimension of said base ranges between 1 nm and 100 μm. In one embodiment, the length of at least one lateral dimension of said base ranges between 1 nm and 2 mm. In one embodiment, the length of at least one lateral dimension of said base is larger than 1 mm, or larger than 100 μm or larger than 500 μm. In one embodiment, the length of at least one lateral dimension of said base ranges between 100 μm and 2 mm, or between 10 μm and 2 mm, or between 100 μm and 10 mm, or between 1 μm and 10mm. In one embodiment, the height of said base ranges between 1 nm and 1 μm. In one embodiment, the height of said base ranges between 1 nm and 100 nm or between 1 nm and 10nm. In one embodiment, the first portion of said base is covered by said nanospikes or nano-protrusions and a second portion of said base is not covered by said nanospikes or nano-protrusions. In one embodiment, at least one portion of the base is covered by said nano-protrusions. In one embodiment, the base comprises at least one facet. In one embodiment, the at least one facet is covered by said nanospikes. In one embodiment, at least one facet is covered by said nanospikes and other facet(s) is/are not covered by said nanospikes. In one embodiment, one portion of said at least one facet is covered by said nanospikes and another portion of said facet is not covered by said nanospikes. In one embodiment, the inner area on the surface of said facet is covered by said nanospikes and the outer perimeter area of said facet is not covered by said nanospikes. In one embodiment, the flattened shape assumes a hexagonal, rectangular, circular, oval, triangular or cylindrical shape. In one embodiment, the number of nanospikes of each nanostructure ranges between 2 and 1,000,000. In one embodiment, the spacing between the nanospikes ranges between 1 nm and 100 nm. In one embodiment, the noble metal comprises gold. In one embodiment, the alloy is a gold alloy. In one embodiment, the noble metal is selected from Ru, Rh, Pd, Ag, Re, Os, Ir and Pt. In one embodiment, the alloy is selected from alloys comprising Ru, Rh, Pd, Ag, Re, Os, Ir and Pt. In one embodiment, the length of said nanospikes ranges between 1 nm and 10 μm. In one embodiment, the base of said nanostructure is attached to a substrate. In one embodiment, the nanostructure is at least partially embedded in said substrate.

In one embodiment, the nanostructure is crystalline. In one embodiment, the nanostructure is a single crystal. In one embodiment, the nanostructure is polycrystalline. In one embodiment, where the nano-protrusions are grown on a film, the film is polycrystalline. In one embodiment, where the nano-protrusions are grown on an evaporated gold film, the evaporated gold film is polycrystalline.

In one embodiment, this invention provides an array of nanostructures attached to a substrate, said nanostructures comprises the nanostructures as described herein. In one embodiment, the substrate comprises silicon dioxide. In one embodiment, the silicon dioxide comprises glass. In one embodiment, the spacing between adjacent nanostructures ranges between 1 nm and 10 μm.

In one embodiment, this invention provides a system for performing analysis of an analyte, the system comprising:
an array of nanostructures attached to a substrate, the nanostructures comprise the nanostructure as described herein, wherein the analyte is bound directly or indirectly to the nanostructure;
a light source;
a light detector;
a processor;
   wherein the light source is configured to irradiate the array, the detector is configured to detect optical signal obtained from the array and the processor is configured to process the signal to yield analytical result.

In one embodiment, the analyte comprises a biomolecule, a nanoparticle, a biological cell or any combination thereof. In one embodiment, the biomolecule comprises a peptide, a protein, nucleic acid, DNA, RNA, a sugar molecule, proteoglycan, glycoproteins, a lipid, a carbohydrate, a fatty acid or a combination thereof. In one embodiment, the RNA is mRNA, siRNA, miRNA, tRNA, rRNA, snRNA. In one embodiment, the RNA is single stranded, double stranded or a combination thereof. In one embodiment, the DNA is nuclear or mitochondrial. In one embodiment, the DNA is double stranded or single stranded. In one embodiment, the analyte comprises other types of nucleic acids.

In one embodiment, the analyte comprises an atom, an ion, a molecule, a chemical compound, a biomolecule, a nucleic acid, a nanoparticle, a biological cell, a component of a biological cell, a toxin, a polymer, a perfluoro-molecule or any combination thereof.

In one embodiment, the analysis is colorimetric analysis. In one embodiment, analysis is an optical analysis. In one embodiment, analysis is carried out using electrochemical techniques. In one embodiment, the analysis is a spectroscopic analysis. In one embodiment, the spectroscopy comprises IR, circular dichroism (CD), Raman, UV, visible, fluorescence, tip-enhanced techniques, or any combination thereof. In one embodiment, the spectroscopy is surface enhanced spectroscopy (SES). In one embodiment, the Raman spectroscopic analysis is SERS.

In one embodiment, this invention provides a system for performing catalysis of a chemical reaction, said system comprising:
an array of nanostructures attached to a substrate, said nanostructures comprises the nanostructure as described herein;
a container in which the array is placed;
means for introducing gas or liquid into the container, the gas or liquid comprise atoms, ions, molecules or any combination thereof;
optionally a heater;
optionally a pump;
   wherein the atoms, ions or molecules are brought into direct or indirect contact with the nanostructures such that a chemical reaction involving the atoms, ions or molecules is catalyzed by the nanostructures.

In one embodiment, the container is selected from a vessel, a cup, a dish, a tube, a tank, a chamber, a conduit or any combination thereof. In one embodiment, the rate of the chemical reaction is higher than the rate of the same reaction wherein the atoms, ions or molecules are not brought into direct or indirect contact with the nanostructures.

In one embodiment, this invention provides a method of preparation of the nanostructures as described herein, the method comprising:
depositing metal from a vapor phase on a substrate to form a metal-coated substrate wherein the metal-coating is in the form of metal islands, non-uniform metal film, a continuous film or any combination thereof;
optionally annealing said metal-coated substrate to form metal islands on the substrate, the metal islands comprise the nanostructure's base;
depositing metal atoms from a liquid solution comprising metal ions, onto the metal-coated substrate, thus forming the nanospikes on the island base.

In one embodiment, the metal is gold. In one embodiment, the annealing is performed at a temperature above the glass transition temperature (T_{g}) of the substrate. In one embodiment, the substrate is or comprises glass. In one embodiment, the substrate is selected from silicon, silicon oxide or silicon coated by silicon oxide. In one embodiment, the substrate comprises glass, borosilicate glass or quartz. In one embodiment, the substrate is coated. In one embodiment, the substrate is coated by a coating layer. In one embodiment, the coating layer is a buffer layer or an adhesion layer. In one embodiment, the substrate is at least partially coated by Cr, Ti or TiO₂. In one embodiment the buffer/adhesion layer improves adhesion of the subsequently deposited metal (e.g, gold) to the substrate.

In one embodiment, the metal atoms are the same metal as the metal ions. For example, metal atoms are formed from metal ions present in the liquid solution. In one embodiment, the metal ions in the liquid solution, receive electrons and form electrically neutral metal atoms. The neutral metal atoms deposit on the surface of the metal islands in one embodiment. Deposition of metal atoms on the metal that coats the substrate generates protrusions on the metal-coating in one embodiment.

In one embodiment, this invention provides a method of preparation of the nanostructures as described herein, the method comprising:
depositing metal from a vapor phase on a substrate to form a metal-coated substrate wherein the metal-coating is in the form of metal islands, non-uniform metal film, a continuous film or any combination thereof;
optionally annealing said metal-coated substrate to form metal islands on the substrate, the metal islands comprise the nanostructure's base;
depositing metal atoms from a liquid solution comprising metal ions, onto the metal-coated substrate, thus forming nanospikes on the metal coating.

In one embodiment, the metal-coating is in the form of metal islands. In one embodiment, the metal-coating comprises metal islands. In one embodiment, the metal-coating consists of metal islands.

In one embodiment, this invention provides a method of preparation of the nanostructures as described herein, the method comprising:
providing a metal-coated substrate wherein the metal-coating is in the form of metal islands, non-uniform metal film, a continuous film or any combination thereof;
optionally annealing the metal-coated substrate;
depositing metal atoms from a liquid solution comprising metal ions, onto the metal-coated substrate, thus forming nano-protrusions on the metal coating.

In one embodiment, this invention provides a method of preparation of nanostructures, the nanostructures comprising:
a metallic base;
metallic nano-protrusions comprising a first end and a second end;
   wherein, said first end is attached to said base and said second end is exposed to the environment; and
   wherein said metallic base and said metallic nano-protrusions comprise a noble metal or an alloy comprising a noble metal; wherein the method comprising:
depositing metal from a vapor phase on a substrate to form a metal-coated substrate wherein the metal-coating is in the form of metal islands, non-uniform metal film, a continuous film or any combination thereof;
optionally annealing said metal-coated substrate to form metal islands on the substrate, the metal islands comprise the nanostructure's base;
depositing metal atoms from a liquid solution comprising metal ions, onto the metal-coated substrate, thus forming nanospikes on the metal coating.

In one embodiment, this invention provides an analysis method comprising:
providing an array of nanostructures attached to a substrate, the nanostructures comprise the nanostructures as described herein;
optionally binding a linker to said nanostructures;
bringing the array in contact with a liquid solution or with a vapor phase comprising an analyte molecule, thus enabling binding of the analyte molecule to the linker or to the nanostructure;
analyzing the analyte.

In one embodiment, the analyzing step comprises:
impinging radiation onto the array from a radiation source;
detecting a radiation signal obtained from the nanostructure, from the analyte, from the linker or from a combination thereof, using a detector;
processing said detected radiation signal, thus analyzing the analyte molecule.

In one embodiment, the analyzing step comprises:
impinging radiation onto the array from a radiation source;
detecting a radiation signal obtained from the substrate, from the nanostructure, from the analyte, from the linker or from a combination thereof, using a detector;
processing said detected radiation signal, thus analyzing the analyte molecule.

In one embodiment, the analyte comprises a biomolecule, a nanoparticle, a biological cell, a cell component, or any combination thereof. In one embodiment, the biomolecule comprises a peptide, a protein, an enzyme, nucleic acid, nucleic acid, DNA, RNA, a sugar molecule, proteoglycan, glycoproteins, a lipid, a fatty acid or a combination thereof. In one embodiment, the RNA is mRNA, siRNA, miRNA, tRNA, rRNA, snRNA. In one embodiment, the RNA is single stranded, double stranded. In one embodiment, the DNA is nuclear or mitochondrial. In one embodiment, the DNA is double stranded or single stranded. Any other types of nucleic acids are included in embodiments of this invention. In one embodiment, the analysis is a spectroscopic analysis. In one embodiment, the spectroscopy is IR, circular dichroism (CD), Raman, UV. visible, fluorescence or any combination thereof. In one embodiment, the Raman spectroscopic analysis is surface enhanced Raman spectroscopy (SERS). In one embodiment, the analysis is a colorimetric analysis.

In one embodiment, this invention provides a catalysis method comprising:
providing an array of nanostructures attached to a substrate, the nanostructures comprise the nanostructure as described herein above;
optionally binding a linker to the nanostructures;
bringing the array in contact with a liquid solution or with a vapor phase comprising species such as atoms, ions, molecules or any combination thereof, thus enabling direct or indirect binding of the species to the linker or to the nanostructure, such that a chemical reaction involving said atoms, ions or molecules is catalyzed by the nanostructures.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1: FIG. 1 is a scheme of Au nanostars formation.

FIG. 2: FIG. 2A is a schematic representation of SERS transducer formation based on Au nanostars formed directly on solid-state (glass) substrates; HR-SEM images of as-deposited Au films (FIG. 2B-Figure 2E), Au films annealed at 580° C for 10 h (FIG. 2F-FIG. 2I), and nanostars formation (FIG. 2J-FIG. 2M). Nominal mass thicknesses of Au: (B, F, J) 1 nm, (C, G, K) 3 nm. (D. H. L) 5 nm and (E, I, M) 7 nm. Scale bars in all images are 100 nm. In the inset images of J-M the scalebar is 20 nm.

FIG. 3: Area fraction (%) and NP density (μm²) (number of particles per square micrometer) (FIG. 3A); Major diameter (nm) and Aspect ratio (AR) (FIG. 3B) vs. Au film thickness (nm) during evaporation of Au NPs annealed at 580 C. for 10 h. The calculation of the parameters has been performed in ImageJ 1.5 image analysis software by means of establishing a color threshold range of the objects apart from the background followed by automatic calculation of the desired parameters; AR is the ratio between the major axis and the minor axis of the particle. NP=nanoparticle.

FIG. 4 UV-Vis extinction spectra of (FIG. 4A) 1, 3, 5, 7-nm-thick Au films deposited on glass substrates and annealed at 580° C. for 10 h, and (FIG. 4B) the same films after nanostar formation. Insets: digital images of the samples.

FIG. 5: TEM images of bright (FIGS. 5A-5E) and dark field (FIGS. 5F-5J) of gold nanostars of 5 nm gold evaporation thickness lifted off from the glass and transferred to a TEM grid.

FIG. 6A-6E: FIG. 6A shows a schematic of Rhodamine 6G (R6G) monolayer adsorbed on the surfaces of gold nanostars. FIGS. 6B shows SERS spectra of Rhodamine 6G (R6G) monolayer adsorbed on the surfaces of gold nanostars (NS) of 1, 3, 5, 7 nm of nominal gold evaporation thickness, a demonstration of surface Raman signal appearance and enhancement upon adsorption of Rhodamine 6G (R6G) on gold nanoislands and on spiked gold structures. FIG. 6C shows an extended spectrum for gold nanostars of 7 nm of nominal gold evaporation thickness. FIG. 6D shows a comparison of SERS spectrum for R6G monolayers adsorbed on gold nanostars compared to a control. FIG. 6E shows transmission spectra for Rhodamine 6G on gold nanoislands and nanostars. FIG. 6F shows the spectrum of FIG. 6E but at a different x-axis scale. FIG. 6G shows transmission spectrum of R6G on gold surfaces. FIG. 6H shows the extinction maximum for different gold+R6G configurations with FIG. 6I being the same but with the y-axis being in a.u.

FIG. 7: Circular dichroism (CD) spectra of the (−)Riboflavin monomolecular layer adsorbed directly on nanospiked gold substrates of 7 nm of nominal gold evaporation thickness before (FIG. 7A) and after (FIG. 7B) glass baseline subtraction. FIG. 7C shows the dependence of the CD spectra of the (−)Riboflavin monomolecular layer adsorbed of 1, 3, 5 and 7 nm of nominal gold evaporation thickness.

FIG. 8: HR-SEM images of Au patterns at different magnifications (FIGS. 8A-8E).

FIG. 9: FIG. 9A shows a digital photo of the slides prepared with a pattern. SEM images of patterns made of Au nanoislands on glass substrates; FIGS. 9B-FIG. 9F are ‘negative’ images; FIG. 9G-FIG. 9K are ‘positive’ images; shadow masks for patterning were made of SU8 (Kayaku-Microchem. USA) photoresist with a thickness range of 150-300 μm. The masks were either placed on a substrate and sticked to it with Capton (or Scotch tape) or assembled in a specially designed holder providing good hard contact between mask and substrate.

FIGS. 10A-101: Scanning electron microscopy (SEM) images of large-scale gold nano-hedgehog samples, illustrating variety of spike sizes, distribution and density on the primary gold structure. Scale bars are 200 nm.

FIG. 11. FIG. 11A shows a schematic representation of spiked gold structure formed on the glass surface. FIG. 11B shows digital images of the samples showing color variations of spiked gold nanostructures formed on the glass slide after 1, 3, 5, 7-nm-thick Au films evaporation respectively on the glass substrates, and after high temperature annealing. FIGS. 11C-11F show SEM images of the four samples presented in FIG. 11B. Scale bars for all images are 100 nm. The scale bar of all insets is 20 nm.

FIG. 12. FIGS. 12A. 12F and 12K show digital images of samples showing color variations with respect to spike-formation conditions of spiked gold nanostructures formed on glass slides after 5 nm-thick Au films evaporation and after high temperature annealing. Substrates (slides) are glass substrates. SEM images of the same samples are shown accordingly: FIGS. 12B-12E correspond to FIG. 12A; FIGS. 12G-12J correspond to FIG. 12F; FIGS. 12L-12M correspond to FIG. 12K. For example: the top left slide in FIG. 12A corresponds to the SEM image in FIG. 12B; the top right slide in FIG. 12A corresponds to FIG. 12C. etc.

FIG. 13 Digital images of samples showing transparency of spiked gold samples FIG. 13A: in this image, the slides are placed over a white paper, the white paper is printed with black ink. Although the slides are colored by the nanostar array, the black writing shows through the colored slide; FIG. 13B is an example of intensive color change (refractive index sensitivity) due to refractive index change of the slide after immersion of ethanol droplet; A variety of spiked gold structures that were prepared using a variation of formation conditions are shown in FIG. 13C-FIG. 13E; The SEM images of the structures were obtained by means of simple placing of gold islands slides of 3, 5 and 7 nm of nominal gold thickness into a beaker with gold precursor salt and HEPES with or without magnet stirrer (procedure further detailed herein below). The different images show the diversity of spikes density. dimensionalities and inter-spike/inter-particle distances obtained when formation conditions are varied.

FIG. 14 TEM images of dry-off sample of gold nanostructures and electron energy loss distribution map. The images show dry take off sample of nanostars (scratched from a slide) transferred onto TEM grid and imaged. The enlarged region in FIG. 14A is the ‘spectrum image’. The images show electron energy loss (EELS) map during electron beam interaction with nanostructures. FIG. 14B is an enlarged image of FIG. 14A. Colors (FIG. 14C) show energy loss of electrons coming from a gun upon interaction with electron cloud of nanostructures and corresponding matter (or de Broglie) wavelength. FIG. 14C shows electron energy loss spectra (EELS) showing different plasmon modes at different regions of scanning.

FIGS. 15A-15C Finite elements electromagnetic field distribution simulation comparison of gold nanoparticle and gold nanostar partially embedded into a glass structure when changing wavelength of incident light; bar on left and right represents electromagnetic field intensity. FIG. 15A is for 535 nm; FIG. 15B is for 635 nm; FIG. 15C is for 785 nm.

FIG. 16. Finite elements electromagnetic field distribution simulation comparison of gold nanostar (NS) partially embedded into glass structure when changing the gap between the structures; FIG. 16A-16B are for 535 nm; FIG. 16C-16D are for 635 nm; FIG. 16E-16F are for 785 nm; WG=wide gap, NG=Narrow gap; and (FIG. 16G) simulated spectra of these systems in comparison to embedded gold nanoparticle (NP).

FIG. 17 Examples of LSPR spectra change of layer-by-layer polyelectrolyte (PE) self-assembly on spiky gold transducers; FIG. 17A and FIG. 17B represent two different samples. Difference in the spectra demonstrates excitation of transverse mode (short wavelength peak) of the nanoparticle electron cloud oscillation, and also longitudinal (long wavelengths); spectrum variations correspond to nanostar geometry change.

FIGS. 18A-18E SEM images of examples of large gold nano-hedgehogs. A demonstration of the effect of shape-independent spikes growth and spikes surface density.

FIG. 19A schematic representation of the principle of protein adsorption and monolayers self-assembly on spiked gold structures. Proteins demonstrated are BSA and Lysozyme.

FIGS. 20 An example of LSPR spectra change of bare gold (black, Mostly lower line) upon interaction with proteins (BSA, FIGS. 20A-20B, and Lysozyme, FIGS. 20C-20D) for two spiked gold systems.

FIG. 21A demonstration of ability to form spiked structures of gold on conductive surfaces (FTO): FIG. 21A: digital image of (from left to right) FTO, FTO after gold evaporation, FTO after high temperature annealing, FTO after spikes formation. FIG. 21B: resistance measurements of FTO glass with formed nanospiked gold.

FIG. 22A schematic representation of DNA hybridization scheme on spiked gold structures (FIG. 22A) and corresponding Raman spectra (FIG. 22B) SEQ ID NO. 1, Anchor ssDNA-Anti-Lysozyme Aptamer:

5′-TTTTTTATCAGGGCTAAAGAGTGCAGAGTTACTTAG-3′-SH.

SEQ ID NO. 2, Complementary analyte ssDNA:

5′-CTAAGTAACTCTGCACTCTTTAGCCCTGATAAAAAA-3′. See right side of image in FIG. 22A.

FIG. 23A demonstration of nanospiked gold application for CD analysis, example of schematic representation of riboflavin binding to the nanostars (FIG. 23A) and corresponding CD spectra (FIG. 23B).

FIG. 24A schematic representation of nanospiked gold application for avidin-biotin binding.

FIGS. 25A-25E An example of spiked gold structures to be applied in pattern formation using shadow mask during gold evaporation.

FIGS. 26A-26F SEM images of patterned spiked structures of gold at different magnifications.

FIG. 27 An example showing a schematic of spiked structures of gold integrated with microfluidic devices (FIG. 27A) and a digital photograph of a number of such microfluidic devices (FIG. 27B).

FIGS. 28A-28B shows photographs of slides prepared under different treatment conditions. FIG. 28A shows a comparison of the slide's transparency and color for pure glass, after 1 Å of gold evaporation thickness and after spikes formation via dipping procedure; FIG. 28B shows a comparison of the slides (1 Å of gold evaporation thickness) obtained via two different procedures, dipping (left) and rotation (right).

FIGS. 29A-29D shows SEM images at different magnification of the gold structures with spikes grown at the slide of 1 Å of gold evaporation thickness using dipping procedure.

FIG. 30 shows photographs of slides prepared with different substrates. FIG. 30A shows slides prepared on borosilicate glass substrates with thicknesses of between 100 μm to 2 mm (from top to bottom). FIG. 30B shows slides prepared on FTO substrates (200 μm). FIG. 30C shows slides prepared on silicon substrates with 250 nm oxide. FIG. 30D shows slides prepared on quartz substrates. FIG. 30E shows slides prepared with silicon substrates with native oxides.

FIG. 31A and 31B shows high-angle annular dark-field (HAADF) scanning transmission electron microscopy (STEM) images of nanostars transferred onto TEM grid at different magnifications. FIG. 31C shows an example of energy-dispersive X-ray spectroscopy (EDS) of nanostars transferred onto a TEM grid. Composition is as expected, Fe and Co are background signal from the sample holder, Cu originates from the TEM grid.

FIG. 32 shows a comparison between nanoislands and nanostars for layer-by-layer polyelectrolyte (PAH/PSS) self-assembly and transducers sensitivity comparison. UV-visible extinction spectra for nanoislands (FIG. 32A) or nanostars (FIG. 32B) with increasing number of polyelectrolyte layer. Change in wavelength peak (FIG. 32C), extinction peak (FIG. 32D) comparing nanoislands to nanostars; UV-visible extinction spectra for bare gold with increasing number of polyelectrolyte layers (FIG. 32E).

FIG. 33 shows configuration used for comparison study of SERS and CD on amyloid fibers formation, folding-unfolding dynamics and ligand binding investigation. This configuration was also used for tip-enhanced Raman spectroscopy (TERS) and a single giant nano-hedgehog.

FIG. 34 shows a scheme for a comparative study of antigen-antibody interaction on surface-tethered nanostars using gold nanoislands system as comparison system. The figure depicts the application of the interface for SERS sensing on lysozyme-anti-lysozyme model systems.

FIG. 35 shows CD spectra of gold nanostars before and after adsorption of riboflavin at different nominal gold thickness evaporation for 1 nm gold nanostar (FIG. 35A), 3 nm gold nanostar (FIG. 35B), 5 nm gold nanostar (FIG. 35C) and 7 nm gold nanostar (FIG. 35D).

FIG. 36 depicts an example scheme demonstrating possibilities of patterns created with gold nanoislands/nanostars using simple lithographic or masking steps.

FIG. 37 depicts another scheme of pattern creation using lithography on pre-existing nanoisland/nanostar structures.

FIG. 38 shows a comparison of the gold island slides (FIG. 38A) and spiked gold structures made with protocol 1 (FIG. 38B) and nano-gold structures made with protocol 2(FIG. 38C), with digital images and corresponding LSPR UV-vis spectra obtained after 1, 3, 5, 7 nm of nominal gold thickness evaporation and high temperature annealing with the following corresponding spectra: FIG. 38D corresponds to the structure presented in FIG. 38A; FIG. 38E corresponds to the structure presented in FIG. 38B; FIG. 38F corresponds to the structure presented in FIG. 38C.

FIG. 39 shows SEM images of different gold nanostructures at different evaporated gold thicknesses. FIG. 39A shows SEM images for 1, 3, 5, 7 nm (‘a’ to ‘d’) Au deposition for random gold islands. FIG. 39B shows SEM images for 1, 3, 5, 7 nm (‘e’ to ‘h’) Au deposition for gold islands. FIG. 39C shows SEM images for 1, 3, 5, 7 nm (‘e’ to ‘h’) Au deposition for nano-structures on gold islands. FIG. 39D shows SEM images for 1, 3, 5, 7 nm (‘i’ to ‘l’) Au deposition for nanostars on gold islands. Scale bars all correspond to 40 nm.

FIG. 40 shows several statistical parameters (number of particles vs. minor and major diameter) for gold islands (FIG. 40A) with respect to the nominal gold evaporation thickness of 1 (FIG. 40B), 3 (FIG. 40C), 5 (FIG. 40D) and 7 nm (FIG. 40E).

FIG. 41 shows a comparison of the statistical distribution parameters (major diameter, area fraction, aspect ratio and islands density) of gold islands (FIG. 41A) with respect to the nominal gold evaporation thickness of 1, 3, 5 and 7 nm for the major diameter (FIG. 41B), the area fraction (FIG. 41C), the aspect ratio (FIG. 41D) and the NP density (FIG. 41E).

FIG. 42 shows several statistical parameters (number of particles vs. minor and major diameter) for gold nano-structures (FIG. 42A) with respect to the nominal gold evaporation thickness of 3 (FIG. 42B), 5 (FIG. 42C) and 7 (FIG. 42D) nm obtained in accordance with the protocol 2, described herein.

FIG. 43 shows a comparison of statistical distribution parameters (major diameter, area fraction, aspect ratio and islands density) for gold nano-structures shown in FIG. 42A with respect to the nominal gold evaporation thickness of 3, 5 and 7 nm obtained in accordance with the protocol 2, described herein, for the major diameter (FIG. 43A), area fraction (FIG. 43B), aspect ratio (FIG. 43C) and NP density (FIG. 43D).

FIG. 44 shows LSPR spectra of gold islands/nanostars before and after lysozyme adsorption (schematic in FIG. 44A) for different gold evaporation thickness of 1, 3, 5 and 7 nm, for relative LSPR peak shift (FIG. 44B), relative peak intensity shift (FIG. 44C) and enhancement factor (FIG. 44D).

FIG. 45 shows LSPR spectra of gold islands/nanostars before and after RSF adsorption (schematic in FIG. 45A) for different gold evaporation thickness of 1, 3, 5 and 7 nm for relative LSPR peak shift (FIG. 45B), relative peak intensity shift (FIG. 45C) and enhancement factor (FIG. 45D).

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Nanostructures of this Invention

In one embodiment, this invention provides a nanostructure comprising:
a metallic base;
metallic nano-protrusions comprising a first end and a second end;
   wherein, said first end is attached to said base and said second end is exposed to the environment; and
   wherein said metallic base and said metallic nano-protrusions comprise a noble metal or an alloy comprising a noble metal.

In one embodiment, this invention provides a nanostructure comprising:
an isolated metallic base;
metallic nano-protrusions comprising a first end and a second end;
   wherein, said first end is attached to said base and said second end is exposed to the environment; and
   wherein said metallic base and said metallic nano-protrusions comprise a noble metal or an alloy comprising a noble metal.

In one embodiment, the shape of said nano-protrusions is selected from spikes, rounded structures, rods, balls, semi-spheres, domes, squares, rectangles, oval, irregular-shaped protrusions or any combination thereof. In some embodiments the terms “spike”, “nano-spike”, “nano-protrusions” and “protrusions” are used interchangeably.

In one embodiment, the shape of the base is selected from spikes, rounded structures, rods, balls, domes, semi-spheres, squares, rectangles, quadrangles, pentagon, hexagon, triangle, oval, irregular-shaped or any combination thereof.

In one embodiment, this invention provides a nanostructure comprising:
a metallic base;
metallic nanospikes comprising a first end and a second end;
   wherein, said first end is attached to said base and said second end is exposed to the environment; and
   wherein said metallic base and said metallic nanospikes comprise a noble metal or an alloy comprising a noble metal.

In one embodiment, this invention provides a nanostructure comprising:
an isolated metallic base;
metallic nanospikes comprising a first end and a second end;
   wherein, said first end is attached to said base and said second end is exposed to the environment; and
   wherein said metallic base and said metallic nanospikes comprise a noble metal or an alloy comprising a noble metal.

In one embodiment, the length of at least one dimension of said nanostructure ranges between 1 nm and 2 mm. In one embodiment, the length of at least one dimension of said nanostructure ranges between 1 nm and 100 μm. “Dimension” can refer to any spatial dimension as related to a particular shape of a nanostructure. For example, the length of “at least one dimension” of a rod-like nanostructure can refer to its radius, diameter or length. Whereas for a nanospike “at least one dimension” can refer to the diameter/radius of the conic base, the length/height of the spike or the width of the tip of the spike. As used herein “protrusion” refers to a structure that extends beyond or above the surface of another structure. In one embodiment, “nano-protrusions” refer to protrusions on the nanoscale. In one embodiment, the noble metal comprises gold. In one embodiment, the noble metal consists of gold. In one embodiment, the alloy is a gold alloy. In one embodiment, the noble metal is selected from Ru, Rh, Pd, Ag, Re, Os, Ir and Pt. In one embodiment, the alloy is selected from alloys comprising Ru, Rh, Pd, Ag, Re, Os, Ir and Pt. In one embodiment, the alloy comprises at least one of Cu, Al. C. In one embodiment, the alloy is an alloy of noble metals. In one embodiment, the noble metal alloy comprises at least two metals selected from Au, Ru, Rh, Pd, Ag, Re, Os, Ir and Pt. In one embodiment, the nanostructure consists of gold. In one embodiment, the nanostructure comprises gold. In one embodiment, the nanostructure comprises gold alloy. In one embodiment, the metallic base and/or the metallic nanospikes consists of a noble metal. In one embodiment, the metallic base and/or the metallic nanospikes consists of an alloy comprising a noble metal. In one embodiment, the metallic base and/or the metallic nanospikes consists of an alloy consisting of noble metals. In one embodiment, the noble metal is silver.

In one embodiment, the base of said nanostructure is attached to a substrate. In one embodiment, the nanostructure is at least partially embedded in said substrate. In one embodiment, the base of the nanostructure is fully embedded in the substrate. In one embodiment, the base comprises a flattened shape, wherein the length of at least one lateral dimension of said shape is larger than the height of said shape. In one embodiment, the base comprises a shape wherein the length of at least one lateral dimension of said shape is smaller than the height of said shape. In one embodiment, the base comprises a non-flattened shape, wherein the length of at least one lateral dimension of said shape is smaller or equivalent to the height of said shape.

In one embodiment, the length of at least one lateral dimension of the base ranges between 1 nm and 100 μm. In one embodiment, the length of at least one lateral dimension of the base ranges between 1 nm and 10 μm, or between 1 nm and 1 μm, or between 1 nm and 500 nm, or between 1 nm and 100 nm, or between 1 nm and 10 nm. In one embodiment, lateral side is a side that is parallel to the substrate. In one embodiment, lateral side is a side that is parallel to the surface of the substrate. In one embodiment, the height of said base ranges between 1 nm and 1um. In one embodiment, the height of said base ranges between 1 nm and 10 nm, or between 1nm and 20 nm, or between 1 nm and 50 nm, or between 1 nm and 100 nm, or between 0.5 nm and 50 nm, or between 1 nm and 10 μm. In one embodiment, the height of the base ranges between 1nm and 100 μm. In one embodiment, the base is in the shape of a rod extending from the substrate with a height ranging between 1 nm and 100 μm. In one embodiment, a first portion of said base is covered by the nanospikes and a second portion of the base is not covered by the nanospikes. In one embodiment, at least one portion of the base is covered by said nano-protrusions. In one embodiment, the base comprises at least one facet. In one embodiment, the at least one facet is covered by said spikes. In one embodiment, one portion of said at least one facet is covered by said spikes and another portion of said facet is not covered by said spikes. In one embodiment, the inner area on the surface of said facet is covered by said spikes and the outer perimeter area of said facet is not covered by said spikes. In one embodiment, the base comprises at least one top facet and at least one side facet. In one embodiment, at least one top facet is covered by spikes while at least one side facet is not covered by spikes. In one embodiment, the top facet(s) are covered by spikes (at least partially) and the side facet(s) are not covered by spikes. In one embodiment, the flattened shape of the base assumes a hexagonal, rectangular, circular, oval, triangular, pentagonal or cylindrical shape.

In one embodiment, the base is spherical. In one embodiment, the base has a rod-like shape. In one embodiment, the base is oval, square, rectangular, tear-drop shaped, dome shaped, cylindrical, cone-shaped, helical, pentagonal or possesses hexagonal feature. In one embodiment, the base is symmetric, and in another embodiment, asymmetric. In one embodiment, the base has high symmetry, and in another embodiment, low symmetry. In one embodiment, the base has no regular shape. In one embodiment, one or more region on the surface of the base are rounded while different one or more regions on the surface of the base are sharp, flat, rough, pointed, cone-shaped or helix-shaped. In one embodiment, the shape of the base consists or comprises of a polygon. In one embodiment, the number of edges of said polygon ranges between 3 and 12. In one embodiment, the edges of the polygon are all equal in length. In one embodiment, at least one edge of said polygon has a length that is different from one or more other edges of the polygon.

In one embodiment, the upper face of the base (the upper face that is not bound to the substrate) is flat. In one embodiment, the upper face is curved. In one embodiment, the upper face is smooth. In one embodiment, the upper face is rough. In one embodiment, the upper face comprises step(s). In one embodiment, a portion of the upper face is rough or smooth, flat or curved, and comprises or does not comprise step(s). In one embodiment, different portions of the upper face have different surface characteristics selected from the characteristics described herein above. In one embodiment, the length of one lateral dimension of the base ranges between 5 nm and 80 nm.

In one embodiment, the spikes or nano-protrusions consist of gold. In one embodiment, the spikes or nano-protrusions comprise gold. In one embodiment, the spikes or nano-protrusions are cylindrical in shape. In one embodiment, the spikes or nano-protrusions are cone shape. In one embodiment, the spikes or nano-protrusions are rod-shaped. In one embodiment, the second end of the spikes or nano-protrusions that is exposed to the environment is tapered. In one embodiment, the second end of the spikes or nano-protrusions that is exposed to the environment is flat. In one embodiment, the second end of the spikes or nano-protrusions that is exposed to the environment is sharp. In one embodiment, the number of spikes or nano-protrusions of each nanostructure ranges between 2 and 1,000,000. In one embodiment, the spacing between the nanospikes or nano-protrusions ranges between 0.5 nm and 100 nm. In one embodiment, the length of said nanospikes or nano-protrusions ranges between 1 nm and 10 μm.

In one embodiment spikes or nano-protrusions of this invention have a length, or a long axis dimension ranging from between 1 nm-1 μm or between 1 nm-100 nm. In one embodiment the spikes or nano-protrusions have a long axis dimension ranging between 1-5 nm, or between 1-10 nm. In one embodiment the spikes or nano-protrusions have a long axis dimension ranging between 10-50 nm, or between 1 nm-50 nm, or between 50-150 nm, or between 10-100 nm or between 100-1000 nm, or between 100-300 nm, or between 300-500 nm, or between 500-700 nm. In one embodiment the spikes or nano-protrusions have a long axis dimension ranging between 10 nm-1000 nm.

In one embodiment, the width, diameter, cross-section or a combination thereof of said nanospikes or nano-protrusions ranges between 0.5 nm and 100 nm, or between 1 nm and 50 nm or between 1 nm and 10 nm, or between 1 nm and 200 nm. In one embodiment, the nanostructure is crystalline. In one embodiment, the nanostructure is a single crystal. In one embodiment, the nanostructure is polycrystalline. In one embodiment, the base of the nanostructure, the spikes or nano-protrusions or a combination thereof is crystalline. In one embodiment, the base of the nanostructure, the spikes or nano-protrusions or a combination thereof is a single crystal. In one embodiment, the base of the nanostructure, the spikes or nano-protrusions or a combination thereof is polycrystalline. In one embodiment, the base of the nanostructure, the spikes or nano-protrusions or a combination thereof is amorphous.

In one embodiment, nanostructures are referred to in short as ‘structures’. In one embodiment, nanospikes are referred to in short as ‘spikes’ or ‘nano-protrusions’.

In one embodiment, second end that is ‘exposed to the environment’ means that the second end is not bound to the base of the nanostructure. In one embodiment, the second end that is ‘exposed to the environment’ means that the second end is not bound to the substrate. In one embodiment, second end that is ‘exposed to the environment’ means that the second end is ‘free’ i.e., it is not bound to the substrate or to the base of the nanostructure. In one embodiment, environment in this context can be for example gas, air, vacuum, liquid, or solution that surrounds the second end of the spikes.

In one embodiment, the base, the spikes the nanostructure or a combination thereof is a particle with the length of at least one dimension being in the nanoscale. In one embodiment, the particle has at least one axis, one dimension, a length, a width, a height, a thickness, a diameter or a combination thereof ranging between 1 nanometer and 1000 nanometers or between 1 nm and 100 nm.

In one embodiment, the base, the spikes or a combination thereof comprise gold alloy. In one embodiment, the material from which the nanostructure, the particle or the surface is made is a gold/palladium alloy. In one embodiment, the material from which the nanostructure, the particle or the surface is made is silver.

In one embodiment, following metal or alloy vapor deposition, metal (or alloy) islands are formed on the substrate. In one embodiment, the metal islands are isolated from one another. In one embodiment, at least some of the metal islands are connected to other islands. In one embodiment, following metal or alloy vapor deposition, a continuous or non-continuous film is formed on the substrate.

In one embodiment, the base structure (the base before application of the nano-protrusions) is obtained by deposition of Au nanoparticles (NPs) on pretreated glass. For example a glass (or other silicon dioxide comprising substrate) is pretreated by adsorption of e.g. amino silane or mercapto-silane to the substrate. Following such adsorption, NP's are deposited on the modified substrate. The same annealing step may be then carried out to stabilize the NP's on the functionalized substrate (i.e. a substrate functionalized by amino-or mercapto-silane or by other silane molecules). According to this aspect and in one embodiment, the formation of the film/islands/base of this invention is not limited to PVD techniques.

In one embodiment, following an annealing (high temperature) step, metal islands are formed on the substrate. In one embodiment, the metal islands are isolated from one another. In one embodiment, following an annealing (high temperature) step, metal islands that were present on the substrate prior to annealing, change at least one of: their dimensions, their size, their shape, their crystallinity, their connectivity to adjacent islands or any combination thereof. In one embodiment, following an annealing (high temperature) step, metal films (continuous or non-continuous) that were present on the substrate prior to annealing, are converted at least partially to isolated metal islands.

Without being bound to any theory, it seems that the gold islands (the bases) serve as “growing seeds”, or cores for spikes/nanoprotrusions growth. Growth of the spikes from Au salt solution on glass was checked, however, no such growth has been observed. It seems that the annealed gold islands serve as “growing seeds”, or cores for the spikes. Glass itself possesses partial negative charge as well as gold nanostructures, obtained by means of surfactant-less liquid solution-based process in their “naked” state. This means that due to electrostatic repulsion, gold nanostructures cannot be obtained by gold reduction from its salt on the bare glass provided in the same solution. This allows extended formation of gold nanostar-based patterns: the selective growth of spikes on gold rather than on the pure glass surface.

In one embodiment, this invention provides a collection of nanostructures of this invention. In one embodiment, this invention provides a powder comprising or consisting of a collection of nanostructures of this invention. In one embodiment, this invention provides a liquid solution, liquid suspension or dispersion comprising collection of nanostructures of this invention. In one embodiment, this invention provides a solid material comprising a collection of nanostructures of this invention. According to this aspect and in one embodiment, the collection of nanostructures of this invention is produced as follows: Initially, nanostructures of this invention are produced as described herein below on a substrate. Following nanostructure formation, the substrate is detached from the nanostructures and the nanostructures are collected. According to this aspect and in one embodiment, this invention provides a method for producing a collection of nanostructures, the method comprises:
formation of nanostructures as described herein on a substrate;
removal of the nanostructures from the substrate.

According to this aspect and in one embodiment, the nanostructures are produced on a glass substrate as described herein. Subsequently, the nanostructures are lifted off the substrates for example by dissolving the glass in an etchant. In one embodiment, separation of the nanostructures from the substrate can be done by soaking the substrate with the nanostructures in 5-40 vol. % HF solution, or in buffered oxide etch solution: (e.g. 6:1 volume ratio of 40% NH4F in water to 49% HF in water) to completely dissolve the glass. Following separation of the nanostructures from the substrate, the nanostructures can be washed and kept in a liquid. In another embodiment, the nanostructures can be washed and dried or dried without washing. Dried collection of nanostructures can be kept as a powder. Dried collection of nanostructures can be kept as a solid material in one embodiment. In one embodiment, the nanostructures are anisotropic. In one embodiment, such nanostructures are Janus-like particles (having spikes on one side and having no spikes on another side).

In one embodiment, nanostructures of this invention can be formed in solution. According to this aspect and in one embodiment, metallic particles are initially prepared/obtained and dispersed in a liquid solution. Next, spikes are formed on the particles from metal ions present in the liquid solution. According to this aspect and in one embodiment, the solution comprising the metallic particles is shaken or stirred while the reaction that produces the spikes takes place. In one embodiment, the solution comprising the metallic particles is mixed, shaken or stirred prior to and/or during and/or after the reaction that produces the spikes takes place. According to this aspect and in one embodiment, this invention provides:

A method of preparation of nanostructures, said nanostructures comprising:
a metallic particle;
metallic nanospikes or nano-protrusions comprising a first end and a second end; wherein, said first end is attached to said particle and said second end is exposed to the environment; and
   wherein said metallic particle and said metallic nanospikes or nano-protrusions comprise
   a noble metal or an alloy comprising a noble metal;
      said method comprising:
providing metal particles in a liquid solution, the liquid solution comprising metal ions;
depositing metal atoms from the liquid solution comprising metal ions, onto said metallic particles, thus forming the nanospikes on the metal nanoparticles.

In one embodiment, the method comprising:
providing metal particles in a first liquid solution;
adding a second liquid solution comprising metal ions to the first liquid solution;
depositing metal atoms from the liquid solution comprising metal ions, onto said metallic particles, thus forming the nanospikes on the metal nanoparticles.

In one embodiment, the method comprising:
providing a first liquid solution comprising metal particles;
providing a second liquid solution comprising metal ions;
mixing the first liquid solution and the second liquid solution;
depositing metal atoms from the liquid solution comprising metal ions, onto said metallic particles, thus forming the nanospikes or nano-protrusions on the metal nanoparticles.

According to this aspect and in one embodiment, the particle is covered by nanospikes or nano-protrusions on all sides. In one embodiment, the particle is isotropic. In one embodiment, the particle is non-isotropic. In one embodiment, all features described herein above for the spikes or protrusions in nanostructures of this invention are applicable to the spikes or protrusions of nanoparticles of this invention as described herein. In one embodiment, features described herein above for the base in nanostructures of this invention are applicable to the nanoparticles of this invention as described herein.

### Films of this Invention

In one embodiment, this invention provides a metallic structure comprising:
a metallic film:
metallic nano-protrusions comprising a first end and a second end;
   wherein, said first end is attached to said metallic film and said second end is exposed to the environment; and
   wherein said metallic film and said metallic nano-protrusions comprise a noble metal or an alloy comprising a noble metal.

In one embodiment, the shape of the nano-protrusions is selected from spikes, rounded structures, rods, balls, domes, squares, rectangles, oval, irregular-shaped or any combination thereof.

In one embodiment, this invention provides a metallic structure comprising:
a metallic film:
metallic nanospikes comprising a first end and a second end;
   wherein, said first end is attached to said metallic film and said second end is exposed to the environment; and
   wherein said metallic film and said metallic nanospikes comprise a noble metal or an alloy comprising a noble metal.

In one embodiment, the film is continuous. In one embodiment, the thickness of the film ranges between 1 nm and 1000 nm, or between 5 nm and 1000 nm, or between 1 nm and 100nm, or between 10 nm and 500 nm. In one embodiment, the noble metal comprises gold. In one embodiment, the noble metal consists of gold. In one embodiment, the alloy is a gold alloy. In one embodiment, the noble metal is selected from Ru, Rh, Pd, Ag, Re, Os, Ir and Pt. In one embodiment, the alloy is selected from alloys comprising Ru, Rh, Pd, Ag. Re, Os, Ir and Pt. In one embodiment, the metallic structure consists of gold. In one embodiment, the metallic structure comprises gold. In one embodiment, the metallic structure comprises gold alloy. In one embodiment, the metallic film and/or the metallic nanospikes or nano-protrusions consists of a noble metal. In one embodiment, the metallic film and/or the metallic nanospikes or nano-protrusions consists of an alloy comprising a noble metal. In one embodiment, the metallic film and/or the metallic nanospikes or nano-protrusions consists of an alloy consisting of noble metals.

In one embodiment, the film of said metallic structure is attached to a substrate. In one embodiment, the film is smooth. In one embodiment, the film is rough. In one embodiment, the height of the film ranges between 1 nm and 1 μm. In one embodiment, a first portion of said film is covered by the nanospikes and a second portion of said film is not covered by the nanospikes. In one embodiment, the film comprises at least one facet. In one embodiment, the at least one facet is covered by said spikes. In one embodiment, one portion of said at least one facet is covered by said spikes and another portion of said facet is not covered by said spikes. In one embodiment, the inner area on the surface of said facet is covered by said spikes and the outer perimeter area of said facet is not covered by said spikes. In one embodiment, the film assumes a quadrangle, a rectangular, a square, a hexagonal, circular, oval, triangular or cylindrical shape. In one embodiment the film comprises more than one shape attached together.

In one embodiment, the film is spherical. In one embodiment, the film has a rod-like shape. In one embodiment, the film is oval, square, rectangular, tear-drop shaped, dome shaped, cylindrical, cone-shaped, helical, pentagonal or possesses a hexagonal feature. In one embodiment, the film is flat. In one embodiment, the film is curved. In one embodiment, the film is wrapped around a curved substrate. In one embodiment, the film is symmetric, and in another embodiment, asymmetric. In one embodiment, the film has high symmetry, and in another embodiment, low symmetry. In one embodiment, the film has no regular shape. In one embodiment, one or more regions on the surface of the film are rounded while other one or more regions on the surface of the film are sharp, flat, rough, pointed, cone-shaped or helix-shaped. In one embodiment, the surface of the film covers an area ranging between 1 nm² and 1 m². In one embodiment, the film covers an area ranging between 1 μm² and 100 cm².

In one embodiment, all features described herein above for the spikes or nano-protrusions in nanostructures of this invention are applicable to the spikes or nano-protrusions in metallic structures comprising films of this invention as described herein. In one embodiment, features described herein above for the nanostructures comprising base and spikes or nano-protrusions are applicable to the metallic structures comprising film and spikes or nano-protrusions in metallic structures of this invention as described herein.

In one embodiment, the film is attached to a substrate. In one embodiment, the substrate comprises silicon dioxide. In one embodiment, the silicon dioxide comprises glass. In one embodiment, the percentage of the surface of the substrate that is covered by the film ranges between 20% and 100% or between 20% and 99.99% or between 1% and 99.99% of the full surface of the substrate. In one embodiment, the percent of the surface of the substrate that is covered by the film is 100%.

In one embodiment, the substrate comprises glass or a glassy material. In one embodiment, the substrate comprises quartz, pyrex, or glass containing any metal ions. In one embodiment, the substrate comprises silicon. In one embodiment, the substrate comprises alumina or silica. In one embodiment, the substrate comprises aluminum coated by aluminum oxide or silicon coated by silicon oxide. In one embodiment, the substrate comprises Ti or TiO₂. In one embodiment, the substrate comprising an amorphous material. In one embodiment, the substrate comprising a crystalline or a semicrystalline material. In one embodiment, the substrate comprising different domains with different crystal structures. In one embodiment, the substrate comprising organic material. In one embodiment the organic material is a polymeric material. In one embodiment, the polymeric material comprises polystyrene, PMMA, PDMS or a combination thereof. In one embodiment, the substrate is flat. In one embodiment, the substrate is curved. In one embodiment, the substrate comprises tin oxide or indium tin oxide or fluorine-doped tin oxide (FTO). In one embodiment, the substrate is electrically-conductive. In one embodiment, the substrate is electrically-insulating. In one embodiment, the substrate is transparent. In one embodiment, the substrate is non-transparent. In one embodiment, the substrate is at least partially transparent. According to this aspect and in one embodiment, the substrate is transparent or semi-transparent at a certain wavelength or a certain wavelength range, and is opaque or semi-transparent at other wavelength(s) or other wavelength ranges.

In one embodiment, the metallic structure comprising the film and the nanospikes is hydrophobic. In one embodiment, the metallic structure comprising the film and the nanospikes is superhydrophobic.

In one embodiment, nanostructures of this invention comprise a base and nanospikes wherein the base and the nanospikes comprise or consist of the same noble metal or the same metal alloy. In one embodiment, nanostructures of this invention comprise a base and nanospikes wherein the base and the nanospikes comprise or consist of different noble metal or different metal alloys.

In one embodiment, structures of this invention comprise a film and nanospikes wherein the film and the nanospikes comprise or consist of the same noble metal or the same metal alloy. In one embodiment, nanostructures of this invention comprise a film and nanospikes wherein the film and the nanospikes comprise or consist of different noble metal or different metal alloys.

In one embodiment, structures of this invention comprise a particle and nanospikes wherein the particle and the nanospikes comprise or consist of the same noble metal or the same metal alloy. In one embodiment, nanostructures of this invention comprise a particle and nanospikes wherein the particle and the nanospikes comprise or consist of different noble metal or different metal alloys.

In one embodiment, for structures comprising gold base, gold film or gold particles, the gold provides a seed for the subsequent formation of gold nanospikes on the base/film/particles, from a liquid solution comprising gold ions. Such seeding mechanism applies to other metals and alloys in structures, particles and films of this invention.

### Arrays of this Invention

In one embodiment, this invention provides an array of nanostructures attached to a substrate, said nanostructures comprise the nanostructure as described herein above.

In one embodiment, this invention provides an array of nanostructures attached to a substrate, said nanostructures comprising:
a metallic base;
metallic nanospikes or nano-protrusions comprising a first end and a second end;
   wherein, said first end is attached to said base and said second end is exposed to the environment; and
   wherein said metallic base and said metallic nanospikes or nano-protrusions comprise a noble metal or an alloy comprising a noble metal.

In one embodiment, the noble metal comprises gold. In one embodiment, the substrate comprises silicon dioxide. In one embodiment, the silicon dioxide comprises glass. In one embodiment, the spacing between adjacent nanostructure in the array ranges between 1 nm and 10 μm. In one embodiment, the spacing between adjacent nanostructure in the array ranges between 1 nm and 1 μm, or between 1 nm and 100 nm, or between 1 nm and 10 nm, or between 0.1nm and 1 μm. In one embodiment, the percent of the surface of the substrate that is covered by the nanostars (nanostructures) ranges between 20% and 99% of the full surface of the substrate. In one embodiment, the percent of the surface of the substrate that is covered by the nanostars ranges between 10% and 30%, or between 10% and 50%, or between 10% and 70%, or between 10% and 90%, or between 20% and 30%, or between 5% and 90%, or between 1% and 99% of the full surface of the substrate. In one embodiment, the percent of the surface of the substrate, in a certain area defined on that substrate that is covered by the nanostars ranges between 20% and 99% of that area defined on the surface of the substrate. In one embodiment, 99.9% of the substrate is covered by the nanostructures.

In one embodiment, the substrate comprises glass or a glassy material. In one embodiment, the substrate comprises quartz, pyrex, or glass containing any metal ions. In one embodiment, the substrate comprises silicon. In one embodiment, the substrate comprises alumina or silica. In one embodiment, the substrate comprises aluminum coated by aluminum oxide or silicon coated by silicon oxide. In one embodiment, the substrate comprises conductive material. In one embodiment, the substrate comprises tin oxide. In one embodiment, the substrate comprises indium tin oxide (ITO). In one embodiment, the substrate comprises fluorinated tin oxide (FTO). In one embodiment, the substrate comprises metal oxide. In one embodiment, the substrate comprises titanium oxide. In one embodiment, the substrate comprising doped silicon or doped silicon oxide.

In one embodiment, the substrate comprises an amorphous material. In one embodiment, the substrate comprises a crystalline or a semicrystalline material. In one embodiment, the substrate comprises different domains with different crystal structures. In one embodiment, the substrate comprises organic material. In one embodiment the organic material is a polymeric material. In one embodiment, the polymeric material comprises polystyrene or PMMA. In one embodiment, the polymeric material comprises organic and inorganic materials. In one embodiment, the polymeric material comprises polydimethylsiloxane (PDMS). In one embodiment, the substrate comprises plastic. In one embodiment, the substrate comprises metal. In one embodiment, the substrate comprises a textile. In one embodiment, the substrate comprises a fabric. In one embodiment, the substrate comprises silk.

In one embodiment this invention provides an array of nanostructures. In one embodiment, an array is a collection of nanostructures. In one embodiment, an array is an assembly of particles (nanostructures). In one embodiment, an array is a structure containing a few or many nanostructures. In one embodiment an array contains 2-10 nanoparticles (nanostructures), or 2-5, 5-10, 10-20, 20-30, 30-50, 10-100, or 100-500 nanoparticles. In one embodiment an array contains 1000-10,000 nanoparticles or 1000-100,000, 1000-1,000,000, 1000-10,000,000, 1000-100,000,000 nanoparticles. In one embodiment an array contains 10-100,000,000 nanoparticles. Any other number-range for nanostructures in arrays of this invention is included in embodiments of this invention.

In one embodiment, an array contains particles of the same size and geometry. In one embodiment, an array contains particles of different size and/or of different geometry. In one embodiment, an array contains particles with a small size distribution (e.g. most particles comprise the same or similar size). In one embodiment, an array contains particles with a large size distribution (e.g. large size differences between different particles in the array).

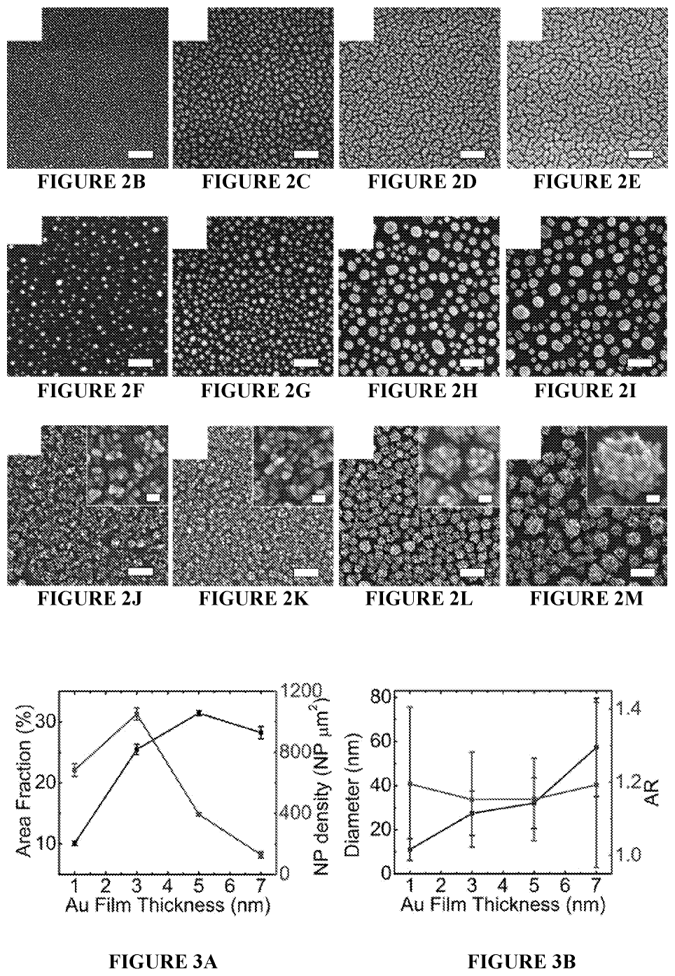

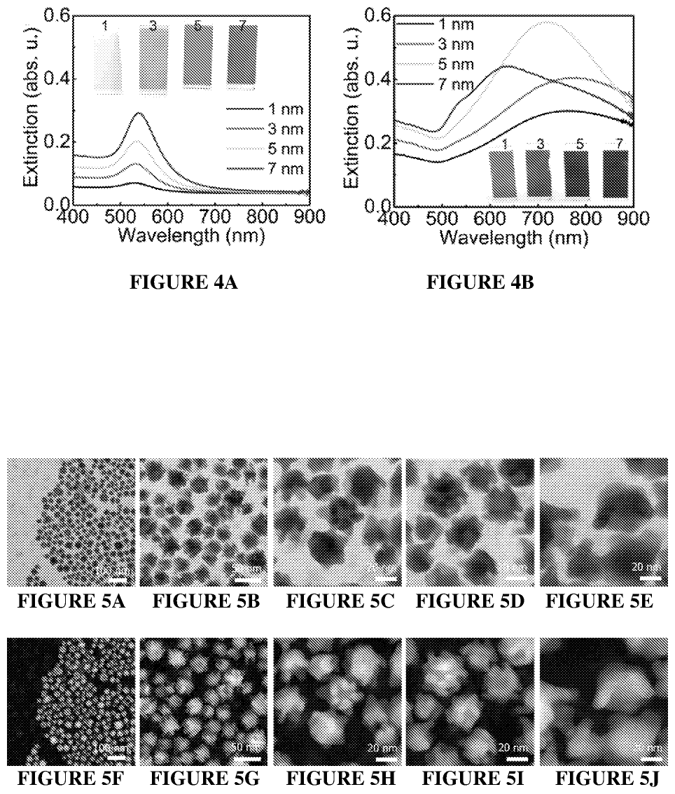

In one embodiment, the absorption spectra of arrays of this invention wherein the nanostructures comprise base and spikes as described herein, is different from the absorption spectra of the same array before application of the spikes, i.e. an array of metal bases (metal islands) before spike addition. According to this aspect and in one embodiment, formation of the spikes changes the color of arrays of this invention as shown for example in FIG. 2A. Absorption spectra described in this embodiment corresponds to UV-Vis spectra in one embodiment.

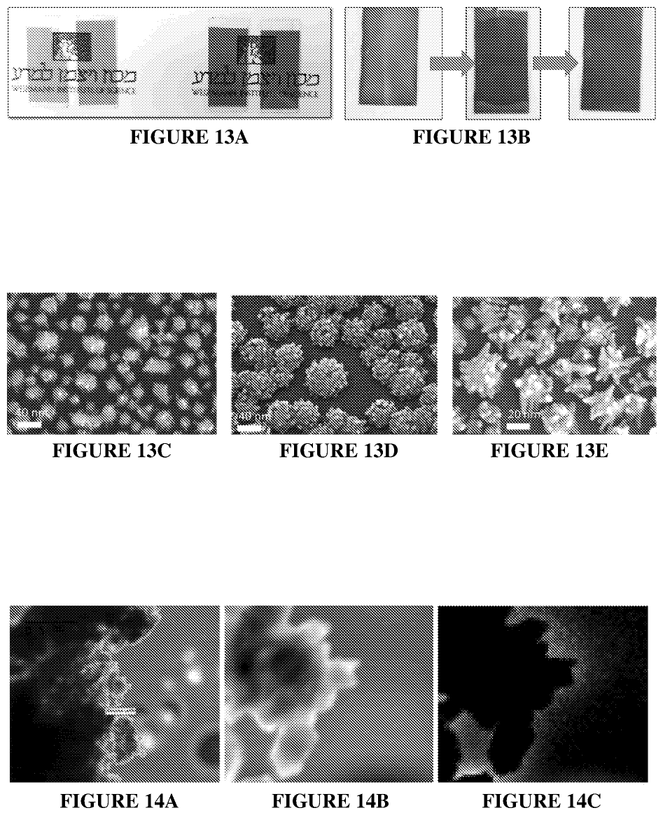

In one embodiment, absorption spectra of arrays of this invention changes when a liquid is applied to the array. According to this aspect and in one embodiment, when liquid is applied to arrays of this invention, the color of the array is changed. This is shown for example in FIG. 13B, where a drop of ethanol was applied to an array of this invention, changing its color from purple/violet as seen in air (RI=1) to blue in ethanol (RI=1.36). Without being bound to any theory, it is believed that this is color change is because LSPR absorbance peak red-shifts from a yellow/green spectral range (530-560 nm) to the red spectral range (>600 nm).

In one embodiment, arrays of this invention have a visible color. In one embodiment, arrays of this invention are transparent. In one embodiment, arrays of this invention have a visible color, however they possess a certain degree of transparency. In one embodiment, colored arrays of this invention allow transmission of light of certain wavelength(s) through the array.

In one embodiment, this invention provides solid-state arrays that are stable, clean (consists of gold and glass only) and that are easily scaled up to very large substrates (e.g. 5-inch diameter wafers). The array parameters can be controlled to have variable surface roughness with plasmonic hot points introduction from the noble metal for a variety of process modifications and process control as required in view of a specific application. In one embodiment, all embodiments described herein for nanostructures of this invention are applicable to nanostructures in arrays of this invention.

### Methods of Preparation of Nanostructures/Arrays of this Invention

In one embodiment, this invention provides a method of preparation of the nanostructure as described herein, the method comprising:
depositing metal from a vapor phase on a substrate to form a metal-coated substrate wherein the metal coating is in the form of metal islands, non-uniform metal film, a continuous film or any combination thereof;
optionally annealing said metal-coated substrate to form metal islands on the substrate, said metal islands comprise said nanostructure's base;
depositing metal atoms from a liquid solution comprising metal ions, onto said metal-coated substrate, thus forming the nanospikes or nano-protrusions on the island base.

In one embodiment, the metal is gold.

In one embodiment, this invention provides a method of preparation of nanostructures, said nanostructures comprising:
a metallic base;
metallic nanospikes or nano-protrusions comprising a first end and a second end;
   wherein, said first end is attached to said base and said second end is exposed to the environment; and
   wherein said metallic base and said metallic nanospikes or nano-protrusions comprise a noble metal or an alloy comprising a noble metal;
   said method comprising:
depositing metal from a vapor phase on a substrate to form a metal-coated substrate wherein said metal coating is in the form of metal islands, non-uniform metal film, a continuous film or any combination thereof;
optionally annealing said metal-coated substrate to form metal islands on said substrate, said metal islands comprise said nanostructure's base;
depositing metal atoms from a liquid solution comprising metal ions, onto said metal-coated substrate, thus forming the spikes on said island base.

In one embodiment, the metal is gold. In one embodiment, the annealing is performed at a temperature above the glass transition temperature (T_{g}) of the substrate. In one embodiment, the substrate is glass. In one embodiment, the substrate comprises glass. In one embodiment, the substrate is selected from silicon, silicon oxide or silicon coated by silicon oxide. In one embodiment, the substrate comprises glass, borosilicate glass, quartz.

In one embodiment, the method of preparation of nanostructures as described herein is also a method of preparation of arrays of nanostructures. Some embodiments related to methods of preparing the nanostructures are applicable to and are included in methods of preparing arrays of nanostructures as described herein.

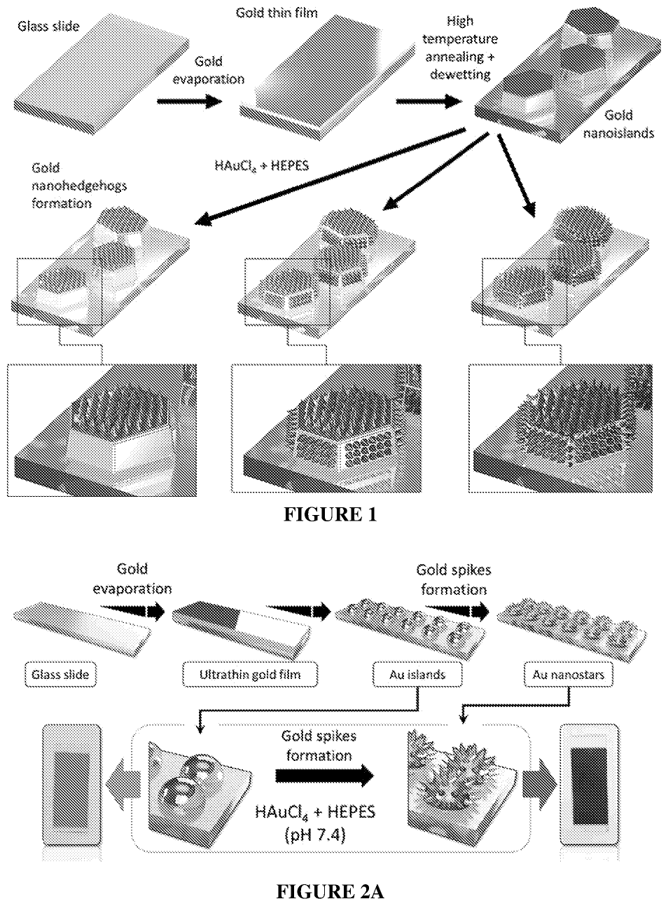

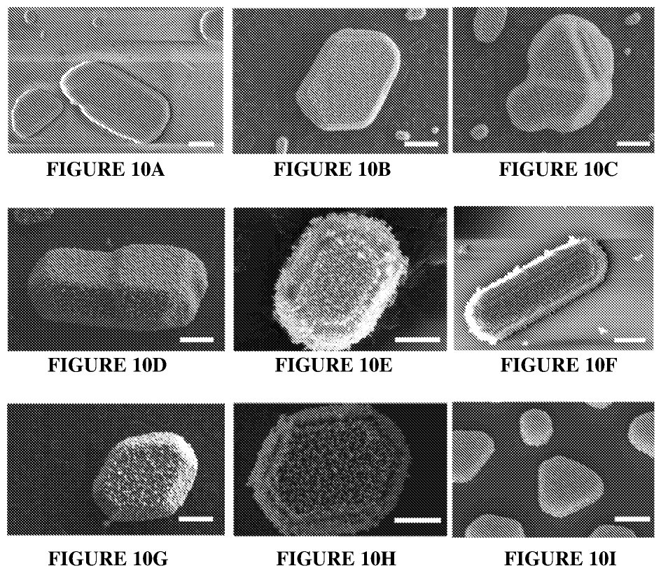

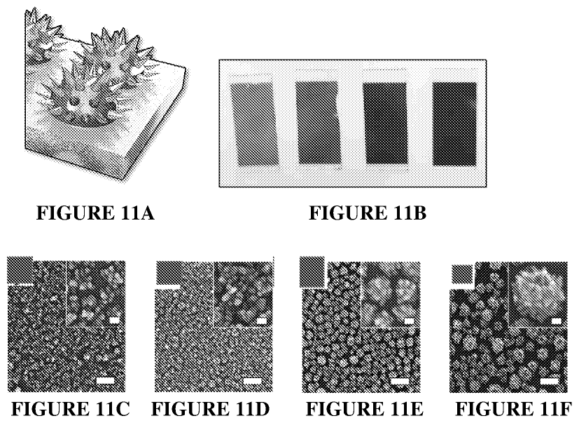

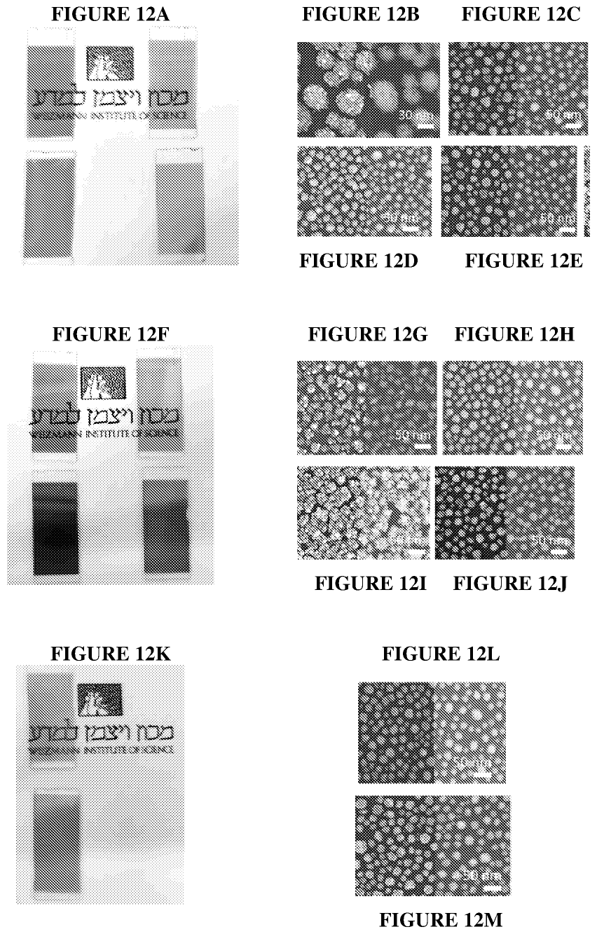

In one embodiment, depositing gold from a vapor phase on a substrate to form a gold-coated substrate comprises the deposition of 1, 3, 5 or 7 nm (nominal gold evaporation thickness as read by the thickness monitor). In general, methods of this invention that are used to form the spiked nanostructures work in a range of thicknesses of deposited metal from 0.1 nm to at least 20 nm (for formation of the base). Example of the nanostructures formed on islands that were formed from gold deposited on a substrate at a thickness higher than 7 nm are shown in FIG. 1 (schematically, facet-selective growth) and in FIG. 10 (SEM images).

In one embodiment, in order to prepare metal films for spikes formation on films, gold films are prepared by depositing between 5 nm and 1000 nm of metal or between 10 nm and 1000nm of the metal on a substrate in some embodiments. In other embodiments, the metal film is provided from another source and the spikes or nano-protrusions are formed on it as described above.

In one embodiment, prior to deposition of the metal (e.g. gold) on the substrate, an adhesion layer or a buffer layer such as Cr or Ti or TiO₂ is deposited onto the substrate to improve stability of the subsequently deposited metal (e.g. gold). Such adhesion layer deposition is also applicable underneath gold films of this invention, films onto which nano-protrusions of this invention are grown.

In one embodiment, the step of ‘optionally annealing said metal-coated substrate’ is conducted not only for the formation of islands, but also or instead for shaping the islands, adjusting the form of the islands, enhancing attachment between the islands and the substrate, embedding or partially-embedding the islands in the substrate, changing the crystallinity of the islands, or any combination thereof. Accordingly, the optional annealing step in embodiments of this invention, refers to any of the aforementioned actions or to any combination thereof.

In one embodiment, the step of “depositing metal atoms from a liquid solution comprising metal ions, onto said metal-coated substrate, thus forming the spikes on the island base” comprises the reduction of metal ions to form the deposited metal atoms. According to this aspect and in one embodiment, the metal ions in the solution and the metal atoms that form the spikes are ions and atoms of the same metal (e.g. gold). The metal ions in the solution form the metal atoms of the spikes. The metal ions in the solution accept electron(s) and are reduced (converted to) the metal atoms that form the spikes in one embodiment.

In one embodiment, variation of parameters of the process of preparation of the nanostructures/films, affect the structural/geometrical and other parameters of the nanostructures/films. According to this aspect and in one embodiment, parameters such as but not limited to: the choice of the substrate, choice of the metal(s), thickness of vapor-deposited metal, deposition rate of vapor deposited metal, annealing time and temperature, content of solution for spike formation and time of the spike-formation step, these parameters may affect nanostructure/film parameters such as but not limited to: nanostructure dimensions, size, shape, geometry, spacing between nanostructures, number of nanostructures, base and spike dimensions, size, shape, geometry, spacing between spikes, number of spikes, nanostructure crystallinity, film roughness, NS array roughness etc. Such variations are included in embodiments of this invention.

In one embodiment, this invention provides nanostructures, particles, films or any combination thereof, prepared by a process of the invention as described herein above.

In one embodiment, methods of preparing the nanostructures, the protrusions, the spikes, or the particles or the films of this invention do not include using a Pb-based catalyst. In one embodiment, methods of preparing nanostructures, protrusions, spikes, particles or films of this invention are electroless. According to this aspect and in one embodiment, methods of preparing nanostructures, protrusions, spikes, particles or films of this invention do not comprise application of a voltage or of an electrical current.

In some embodiments, as described herein, the step of formation of the nano-protrusions (e.g. nanospikes) is conducted using a liquid solution comprising metal ions, wherein the metal ions are reduced to form neutral metal atoms that are deposited on a metal base/film/particle, thus forming a nano-protrusion. In embodiments of this invention, various materials are used in the deposition solution such as ascorbic acid, surfactants and other buffers similar to HEPES.

In one embodiment, an advantage of methods of this invention and of substrates/films of this invention is that the metal on the substrate forms a stable structure, which is coated with Au nano-protrusions particles. According to this aspect and in one embodiment, stability of the functionalized substrate is an advantage of methods and structures of this invention, such stability has not been previously suggested. In one embodiment, an annealing process of gold films on a substrate, enables stabilization of the structures as described herein. In one embodiment, annealing causes embedding of the gold (or other metal) at least partially into the substrate, thus stabilizing the gold film/islands.

In one embodiment, buffers that are suitable to include in the metal-ion solution used for growth of the protrusions are Good's buffers, especially, HEPES. In one embodiment, protrusion growth was obtained with Triton X-100 surfactants. According to this aspect and in one embodiment, protrusion growth was surfactant-supported. In one embodiment, protrusion growth was surfactant-less as in one embodiment of the HEPES-based process.

In one embodiment, various buffer pH values of the protrusion-growth solution can be used in methods of this invention. In one embodiment, other Good's buffers such as MES, HEPPS, MOPC are used in protrusion-growth solutions. For example and in one embodiment, using MOPC, an extremely fine facet-selective surface coverage of a big gold island with spikes was obtained.

In one embodiment, this invention provides a method of preparation of the nanostructures as described herein, the method comprising:
providing a metal-coated substrate wherein the metal-coating is in the form of metal islands, non-uniform metal film, a continuous film or any combination thereof;
optionally annealing the metal-coated substrate;
depositing metal atoms from a liquid solution comprising metal ions, onto the metal-coated substrate, thus forming nano-protrusions on the metal coating.
   According to this aspect and in one embodiment, the base for nanostructures of this invention is provided ready for the step of forming the nano-protrusions. According to this aspect and in one embodiment, the metal-coated substrate (metal film/metal islands coating) is pre-formed. Methods for pre-forming the coated substrate include but are not limited to photolithography, imprinting, e-beam lithography, other mask-based methods, nanosphere lithography, metal evaporation, vapor deposition, CVD, PVD, sputtering, nanoparticle deposition, pattern transfer or any combination thereof. According to this aspect and in one embodiment, methods of this invention comprise depositing metal atoms from a liquid solution comprising metal ions, onto a metal-coated substrate, thus forming nano-protrusions on the metal coating. In one embodiment, this invention provides a method of preparation of the nanostructures as described herein, the method comprising: depositing metal atoms from a liquid solution comprising metal ions, onto a metal-coated substrate, thus forming nano-protrusions on the metal coating. In one embodiment, nanostructures, films and/or particles comprising a base and nano-protrusions are formed by this method.

### Devices and Systems of this Invention

In one embodiment, this invention provides a system for performing analysis of an analyte, said system comprising:
an array of nanostructures attached to a substrate, said nanostructures comprises the nanostructure described herein above, wherein the analyte is bound directly or indirectly to the nanostructure;
a light source;
a light detector;
a processor;
   wherein the light source is configured to irradiate the array, the detector is configured to detect optical signal obtained from the array and the processor is configured to process the signal to yield analytical result.

In one embodiment, this invention provides a system for performing analysis of an analyte, said system comprising:
an array of nanostructures attached to a substrate, said nanostructures comprising:
   metallic base;
   metallic nanospikes comprising a first end and a second end;
      wherein, said first end is attached to said base and said second end is exposed to the environment, and
      wherein said metallic base and said metallic nanospikes comprise a noble metal or an alloy comprising a noble metal; and
      wherein the analyte is bound directly or indirectly to the nanostructure;
a light source;
a light detector;
a processor;
   wherein the light source is configured to irradiate the array, the detector is configured to detect optical signal obtained from the array and the processor is configured to process the signal to yield analytical result.

In one embodiment, the analyte comprises a biomolecule, a nanoparticle, a biological cell or any combination thereof. In one embodiment, the biomolecule comprises a peptide, a protein, an enzyme, nucleic acid, DNA, RNA, a sugar molecule, proteoglycan, glycoproteins, a lipid, carbohydrate, a fatty acid or a combination thereof. In one embodiment, the RNA is mRNA, siRNA, miRNA, tRNA, rRNA, snRNA. In one embodiment, the analyte comprises other types of RNA, DNA or LNA. In one embodiment, the RNA is single stranded, double stranded or a combination thereof. In one embodiment, the DNA is nuclear or mitochondrial. In one embodiment, the DNA is double stranded or single stranded. In one embodiment, the biomolecule comprises a nucleic acid. In one embodiment, the nucleic acid comprises DNA, RNA or derivatives and modifications thereof. In one embodiment, the nucleic acid comprises other sequences. In one embodiment, the nucleic acid is not DNA, RNA or their derivatives. In one embodiment, the analysis is a spectroscopic analysis. In one embodiment, the spectroscopy comprises IR, circular dichroism CD, Raman, UV, visible, fluorescence or any combination thereof. In one embodiment, the spectroscopy is surface enhanced spectroscopy (SES). In one embodiment, the Raman spectroscopic analysis is SERS.

In one embodiment, the analyte comprises an atom, an ion, a molecule, a chemical compound, a biomolecule, a nanoparticle, a biological cell, a component of a biological cell, a toxin, a polymer, a perfluoro-molecule or any combination thereof.

In one embodiment, this invention provides a system for performing catalysis of a chemical reaction, said system comprising:
an array of nanostructures attached to a substrate, said nanostructures comprises the nanostructures as described herein above;
a container in which said array is placed;
means for introducing gas or liquid into said container, said gas or liquid comprise atoms, ions, molecules or any combination thereof;
optionally a heater;
optionally a pump;
   wherein said atoms, ions or molecules are brought into direct or indirect contact with said nanostructures such that a chemical reaction involving said atoms, ions or molecules is catalyzed by said nanostructures.

In one embodiment, this invention provides a system for performing catalysis of a chemical reaction, said system comprising:
an array of nanostructures attached to a substrate, said nanostructures comprising:
   a metallic base;
   metallic nanospikes comprising a first end and a second end;
wherein, said first end is attached to said base and said second end is exposed to the environment; and wherein said metallic base and said metallic nanospikes comprise a noble metal or an alloy comprising a noble metal;
a container in which said array is placed;
means for introducing gas or liquid into said container, said gas or liquid comprise atoms, ions, molecules or any combination thereof;
optionally a heater;
optionally a pump;
   wherein said atoms, ions or molecules are brought into direct or indirect contact with said nanostructures such that a chemical reaction involving said atoms, ions or molecules is catalyzed by said nanostructures.

In one embodiment, the container is selected from a vessel, a cup, a dish, a tube, a tank, a chamber, a conduit or any combination thereof. In one embodiment, the rate of said chemical reaction is higher than the rate of the same reaction wherein said atoms, ions or molecules are not brought into direct or indirect contact with said nanostructures.

### Methods of Use of Nanostructures/Arrays of this Invention

In one embodiment, this invention provides an analysis method comprising:
providing an array of nanostructures attached to a substrate, said nanostructures comprises the nanostructures as described herein above;
optionally binding a linker to said nanostructures;
bringing the array in contact with a liquid solution or with a vapor phase comprising an analyte molecule, thus enabling binding of said analyte molecule to said linker or to said nanostructure;
analyzing said analyte.

In one embodiment, the analyzing step comprises:
impinging radiation onto said array from a radiation source;
detecting a radiation signal obtained from said nanostructure, said analyte, said linker or from a combination thereof, using a detector;
processing said detected radiation signal, thus analyzing said analyte molecule.

In one embodiment, this invention provides an analysis method comprising:
providing an array of nanostructures attached to a substrate, said nanostructures comprising:
   a metallic nano base;
   metallic nanospikes comprising a first end and a second end;
wherein, said first end is attached to said base and said second end is exposed to the environment; and wherein said metallic base and said metallic nanospikes comprise a noble metal or an alloy comprising a noble metal;
optionally binding a linker to said nanostructures;
bringing the array in contact with a liquid solution or with a vapor phase comprising an analyte molecule, thus enabling binding of said analyte molecule to said linker or to said nanostructure;
analyzing said analyte.

In one embodiment, the analyzing step comprises:
impinging radiation onto said array from a radiation source;
detecting a radiation signal obtained from said nanostructure, said analyte, said linker or from a combination thereof, using a detector;
processing said detected radiation signal, thus analyzing said analyte molecule.

In one embodiment, the analyte comprises a biomolecule, a nanoparticle, a biological cell or any combination thereof. In one embodiment, the biomolecule comprises a peptide, a protein, an enzyme, nucleic acid, DNA, RNA, a sugar molecule, proteoglycan, glycoproteins, a lipid, a fatty acid or a combination thereof. In one embodiment, the RNA is mRNA, siRNA, miRNA, tRNA, rRNA, snRNA. In one embodiment, the RNA is single stranded, double stranded. In one embodiment, the DNA is nuclear or mitochondrial. In one embodiment, the DNA is double stranded or single stranded. In one embodiment, the analysis is a spectroscopic analysis. In one embodiment, the spectroscopy is IR, circular dichroism (CD), Raman, UV, visible, fluorescence or any combination thereof. In one embodiment, the Raman spectroscopic analysis is surface enhanced Raman spectroscopy (SERS).

In one embodiment, the step of “detecting a radiation signal obtained from said nanostructure, said analyte, said linker or from a combination thereof, using a detector” refers to radiation transmitted by, reflected from, generated by, refracted, diffracted, or by any mechanism obtained from the nanostructure, the analyte, the linker or from a combination thereof. In one embodiment, the substrate affects this radiation signal and the signal obtained depends on the substrate.

In one embodiment, the description of analysis using an array is applicable for analysis using particles/nanostructures and films of this invention. In one embodiment, arrays, nanostructures and films of this invention, when used for analysis as described herein above, are reusable. According to this aspect and in one embodiment, following analyte binding and detection, the analyte is washed away from the array/structure/particle/film. The array is thus cleaned and ready for adsorption/binding of an additional analyte (same or different analyte).

In one embodiment, analysis methods of this invention comprise measurement of a control sample. According to this aspect and in one embodiment, the array/film is measured (e.g. optically detected) without the analyte, and this measurement is compared to a measurement of the same array/film with the analyte. In some embodiments, the difference between these two measurements yields a qualitative and/or a quantitative result with regards to the nature and/or the quantity of the analyte. In one embodiment, the analysis is a colorimetric analysis. In one embodiment, analysis methods of this invention comprise colorimetric analysis/sensing methods. In one embodiment, colorimetric analysis/sensing means that the optical change is observable by a naked eye.

In one embodiment, this invention provides a catalysis method comprising:
providing an array of nanostructures attached to a substrate, said nanostructures comprises the nanostructures as described herein;
optionally binding a linker to said nanostructures;
bringing the array in contact with a liquid solution or with a vapor phase comprising species such as atoms, ions, molecules or any combination thereof, thus enabling direct or indirect binding of said species to said linker or to said nanostructure, such that a chemical reaction involving said atoms, ions or molecules is catalyzed by said nanostructures.

In one embodiment, this invention provides a catalysis method comprising:
providing an array of nanostructures attached to a substrate, said nanostructures comprising:
   a metallic nano base;
   metallic nanospikes comprising a first end and a second end;
wherein, said first end is attached to said base and said second end is exposed to the environment; and wherein said metallic base and said metallic nanospikes comprise a noble metal or an alloy comprising a noble metal;
optionally binding a linker to said nanostructures;
bringing the array in contact with a liquid solution or with a vapor phase comprising species such as atoms, ions, molecules or any combination thereof, thus enabling direct or indirect binding of said species to said linker or to said nanostructure, such that a chemical reaction involving said atoms, ions or molecules is catalyzed by said nanostructures.

In one embodiment, the array is placed in a container. In one embodiment, the container is selected from a vessel, a cup, a dish, a tube, a tank, a chamber, a conduit or any combination thereof. In one embodiment, the rate of said chemical reaction is higher than the rate of the same reaction wherein said atoms, ions or molecules are not brought into direct or indirect contact with said nanostructures.

In one embodiment, this invention provides a method of increasing the surface area of a structure/film. According to this aspect and in one embodiment, a structure, a particle, a base, an island, or a film is being coated by nanospikes or nano-protrusions, thus increases the surface area of the structure, the particle, the base, the island or the film. According to this aspect and in one embodiment, this invention provides a method of increasing the surface area of a material, the method comprising:
providing a metallic material;
forming metallic nanospikes on the surface of the material, the metallic nanospikes comprising a first end and a second end;
   wherein, said first end is attached to said material and said second end is exposed to the environment; and wherein said metallic material and said metallic nanospikes comprise a noble metal or an alloy comprising a noble metal.

Features of the nanospikes and of the nanostructures/bases/particles/islands/films described herein are applicable to this method of increasing the surface area of a material as described herein above.

In one embodiment, this invention provides a method of roughening the surface of a structure/film. According to this aspect and in one embodiment, a structure, a particle, a base, an island, or a film is being coated by nanospikes, thus roughening the surface of the structure, the particle, the base, the island or the film. According to this aspect and in one embodiment, this invention provides a method of roughening the surface of a material, the method comprising:
providing a metallic material;
forming metallic nanospikes on the surface of the material, the metallic nanospikes comprising a first end and a second end;
   wherein, said first end is attached to said material and said second end is exposed to the environment; and wherein said metallic material and said metallic nanospikes comprise a noble metal or an alloy comprising a noble metal.

Features of the nanospikes or nano-protrusions and of the nanostructures/bases/particles/islands/films described herein are applicable to this method of roughening the surface of a material as described herein above. In embodiments of this invention, formation of the spikes as described herein is used in methods for changing the properties of surfaces of materials. Such properties affected by the formation of spikes on the surface of the material include optical properties, hydrophobicity, geometry, surface area, roughness, affinity toward reagents/analytes, friction, adsorption. Accordingly, methods to induce such and similar changes in the properties of a material are included in embodiments of this invention.

### Coating

In one embodiment, surfaces of the nanostructures or of films or of particles of this invention are covered with a coating layer. In one embodiment, the coating layer is or comprises an organic molecule with at least one functional end group. In one embodiment, the molecule coating the surface is termed a ‘ligand’ or a ‘linker’. In one embodiment, a “ligand” is synonymous with a “molecule”. In one embodiment, the term ‘ligand’ is used because the molecules are bonded around the particle. In one embodiment, the molecules are ligating the surface of the particle/nanostructure. In one embodiment, the term ‘ligand’ is borrowed form coordination chemistry in which a ligand is a molecule coordinated to a metal ion. In coordination chemistry, a ligand is a molecule or an ion having a lone electron pair that can be used to form a bond to a metal ion. In the coated nanoparticle field, the nanoparticle may represent the metal ion, and the molecule coating the nanoparticle may be called a ‘ligand’ in analogy to coordination chemistry. This terminology is known to a person skilled in the art.

In one embodiment the functional end group of the molecule coating the surface or the particle is a thiol group. In one embodiment, a thiol group is a group comprising a sulfur atom bound to a hydrogen atom. In one embodiment, a thiol group is denoted by —SH. In one embodiment the functional end group is a carboxylic acid group. In one embodiment the functional end group is an amine group. In one embodiment, the functional end group serves as the anchor of a rod-like organic molecule to the surface of the structure/particle. Once an anchor is made between the functional end group and the surface or the nanoparticles, neighboring organic molecules that have similar rod-like structure are self-assembled with their long axis perpendicular to, or with a certain tilt angle, with respect to the surface of the material or with respect to the particle surface. The long axis of one molecule is assembled parallel to the long axis of a neighboring molecule. Two molecules are held stretched in this way due to the van der Waals forces between the long “tails” of the molecules. Such arrangement forms a packed mono-molecular layer on the surface of the material or the particle. In one embodiment, “mono-molecular layer” is synonymous with the term “monolayer”. In one embodiment a monolayer is a single layer of organic molecules arranged on the material surface or the particle surface. In one embodiment the long tail of the molecule or a portion of it, is hydrophobic. In one embodiment the long tail comprising a hydrocarbon chain. In one embodiment, the hydrocarbon is an alkane. In one embodiment, the alkane is made of a chain of single-bonded carbon atoms, wherein each carbon atom is bonded to hydrogen atoms as well. In one embodiment, such monolayer is referred to as “self-assembled” monolayer. In one embodiment “self-assembled” means that the monolayer is formed spontaneously from solution onto the surface. In one embodiment, “self-assembled” means that under the right conditions, molecules will approach the structure/particle surface, or any other material surface, will anchor to it, and will stretch their tails by interacting with neighboring anchoring molecules, forming an ordered layer. In one embodiment, “self-assembly” means that the ordered or partially-ordered assembly of molecules was formed without further intervention. In one embodiment, self-assembly of molecules on the surfaces or on the nanoparticles means a spontaneous bonding of molecules to, attraction of molecules to, adsorption of molecules on, association of molecules with, precipitation of molecules on the surfaces/nanoparticles of this invention.

In one embodiment, the coating or self-assembly process results in surfaces/nanoparticles/nanostructure having a coating comprising between 60%-98% of their surface area. In one embodiment, the surfaces/nanoparticles organic-monolayer coating comprising 98%-100% of the particle's surface area. In one embodiment, the surfaces/nanoparticles organic-monolayer coating comprising 98%-99% or 95%-99% or 98%-99.9% of the surface/particle surface area. In one embodiment, the surfaces/nanoparticles organic-monolayer coating comprising 85%-95% or 75%-90% or 40%-60% or 10%-40% of the surface/particle surface area. In one embodiment, the process results in surfaces/nanoparticles having a coating comprising between 50%-100% of their surface area.

In one embodiment, an additional functional group is present on the self-assembled molecules. The additional functional group is located at the molecule end that is exposed to the environment. In one embodiment, this additional functional group is used for chemical reactions/chemical interactions. In one embodiment, this additional functional group is used for analysis. In one embodiment, this additional functional group is used for catalysis. In one embodiment this additional functional group is used for linking other molecules to the surface/particle. In one embodiment this additional functional group is used for linking or for attracting a substrate. In one embodiment, this additional group is an imidazole. In one embodiment this additional functional group is a carboxylic acid, an amine, a biotin, hydroxyl, ethylene glycol, an unsaturated hydrocarbon, or a phenyl. In one embodiment, the additional functional group contains a halogen atom. In one embodiment, the additional functional group contains a metal or a metal ion. In some embodiments the functional group is or resembles the polar end group of natural and synthetic lipids. In one embodiment, the additional functional group comprises a porphyrin, hydroxamate, catechol, EDTA, or other organic and biological ligands or chelating groups. In some embodiments the additional functional groups can be bound to a protein or a DNA molecule. In one embodiment, the additional group comprises an enzyme or a ribozyme. In one embodiment, the additional functional group comprises an enzyme-mimetic molecule. In one embodiment the additional exposed functional group can bind the surface or the nanoparticles to a cell receptor or to a cell membrane. In one embodiment the additional functional group form links between two surfaces/nanoparticles. In one embodiment the additional functional group or the molecule bound to it represent a targeting moiety, for use e.g. in in-vivo applications or in biological assays. In one embodiment the targeting moiety bound through the surface-exposed additional functional groups is used for tissue targeting. In one embodiment the targeting moiety binds to receptors on cells. In one embodiment the targeting moiety adheres to cell membranes. In one embodiment, the additional functional group is or comprises nucleic acid, DNA, RNA, peptide.

In one embodiment a cleavable moiety is bounded through the functional group. In one embodiment cleavable moiety is used for controlled drug release. In one embodiment the functional group or the molecule bound to it is a fluorescent marker. In one embodiment the functional group or the molecule bound to it are used for immunoassays. In one embodiment, engineering of the additional functional groups on the organic molecule (linker) renders the coated surface/particle compatible with various environmental conditions. In one embodiment, choice of the functional group that is exposed to the environment, results in the desired miscibility of the coated surface/particle in aqueous or in organic solvent. In one embodiment, choice of the functional group that is exposed to the environment, results in the desired permeation ability of the coated surface/particle through cell membranes or filters. In one embodiment, choice of the functional group that is exposed to the environment, results in the desired chemical reactivity of the coated surface/particle. In one embodiment, choice of the functional group that is exposed to the environment, results in the desired catalytic activity of the coated surface/particle. In one embodiment, choice of the functional group that is exposed to the environment, results in the desired stability of the coated surface/particle. In one embodiment, choice of the functional group that is exposed to the environment, results in the desired affinity of the coated surface/particle to aqueous or to organic solvents/solutions.

In one embodiment, the coating molecule or the linker can be described as having two portions. One portion is the end group that is attached to the surface of the structure/particle, and another end group that is exposed to the environment. In another embodiment, the coating molecule or the linker can be described as having three portions. One portion is the end group that is attached to the surface of the structure/particle, another portion is the end group that is exposed to the environment, and a third portion is located between these two end groups. The third portion is a long rod-like structure (e.g. alkane or alkene chain or RNA/DNA strand). The functional groups described above may fit the first and the second functional groups (end groups/portions) in embodiments of this invention.

In one embodiment, the functional group is only partially exposed or is not exposed to the environment, until certain conditions such as pH, temperature, ionic concentration, or chemical environment cause the functional group to be exposed to the external environment of the structure/particle.

In one embodiment, a catalyst molecule possesses (i) an anchor group to the surface (of e.g. a particle), (ii) a spacer or a linker, and (iii) a functional group that is responsible for catalysis. In one embodiment, the term catalyst refers to the whole molecule with the three parts. In another embodiment, the term catalyst refers to the functional group only.

In one embodiment, the external end group of the coating molecule is used for binding an analyte for analysis. In one embodiment, the external end group of the coating molecule is used for catalysis. In one embodiment, the catalyst part of the coating molecule comprises an inorganic group such as a metal ion, a metal cluster, a metal oxide particle/cluster. In one embodiment, the catalyst comprises an organo-metallic moiety. In one embodiment, the catalyst comprises an organic moiety.

In one embodiment, the surface of the structure/particle itself is the binder of the analyte with no additional coating molecules attached to it. In one embodiment, the surface of the structure/particle itself is the catalyst with no additional coating molecules attached to it. According to this aspect and in one embodiment, analysis of an analyte is by direct binding of the analyte to the surface of the structure/particle/film. According to this aspect and in one embodiment, catalysis of a reaction proceeds by direct binding of the reactants to the surface of the structure/particle.

In one embodiment a portion of the coating molecules have a hydrophobic terminus that is surface exposed after monolayer formation. In one embodiment the majority of surface exposed hydrophobic groups, makes the particles soluble in an organic solvent. In one embodiment a portion of the organic coating molecules have a hydrophilic end group exposed to the surface after monolayer formation. In one embodiment the hydrophilic molecules may modify or control the total solubility of the particles. In one embodiment, some hydrophilic end groups exposed to the surface enables better solubility of the particles in less hydrophobic organic solvents. In one embodiment, the ratio of hydrophobic to hydrophilic exposed groups on the molecules forming the particle coating, fine-tunes the solubility of the molecules in a certain organic solvent. In one embodiment, if the majority of the exposed end-groups are hydrophilic, or if some of the exposed end-groups are highly hydrophilic, the particle may be soluble in aqueous solutions or in water. In one embodiment, when hydrophobicity/hydrophilicity issues described herein above are addressed to large surfaces instead of to particles, such modifications will modify the wettability of the surfaces by polar/non-polar solvents and will affect the accessibility of the surfaces to species from organic/aqueous solutions, according to the solubility considerations described herein above.

In one embodiment, the process of forming the spikes is done by immersing the metallic surface of the structure/particle/film in solution. In one embodiment, a solution containing a positive metal ion is brought under conditions in which the metal ion is reduced to form the corresponding metal atom (e.g. gold metal ion forms gold metal atom). In one embodiment, the solution contains a reduction agent. In one embodiment, forming metal atoms join together to form a spike. In one embodiment, addition of metal atoms to the spike increases the size of the spike.

In one embodiment, spike synthesis is carried out using an aqueous solution of a gold salt. In one embodiment, the gold salt is HAuCl₄·3H₂O. In another embodiment, spike synthesis is carried out using an organic solution.

In one embodiment, the additional material adsorbed on or coating the surface is biological. In another embodiment, the additional material adsorbed on or coating the surface is a drug or therapeutic agent. In one embodiment the material is a protein. In one embodiment the material is an enzyme or an enzyme-mimetic molecule.

In one embodiment the material is a peptide. In one embodiment the material is a receptor. In one embodiment the material can bind to a receptor. In one embodiment the material is an antibody or an antigen. In one embodiment, the material enables the bonding of a particle to a cell, or bonding of a cell to a surface. In one embodiment bonding of particle to a cell (or a cell to a surface) induces catalysis of a reaction. In one embodiment, the protein is fluorescent. In one embodiment, the biological material is a DNA, RNA, a nucleic acid or a nucleic acid sequence. In one embodiment the additional material is adsorbed onto the surface from solution. In one embodiment adsorption involves covalent bonds. In one embodiment adsorption involves polar, ionic, coordination or van der Waals bonds. In one embodiment adsorption is reversible.

In one embodiment a process of this invention comprises preparing a solution containing ions which will be used to form the spikes. In one embodiment, formation of the spikes is conducted using HEPES buffer solution. In one embodiment, formation of the spikes is conducted using other aqueous solutions. In one embodiment, formation of the spikes is conducted using organic solutions. In one embodiment the solvent in organic solutions used in processes of this invention is toluene. In one embodiment the solvent is benzene, ether or hexane. In one embodiment, the solvent is methanol, ethanol, acetonitrile, DMF, THF, methylene chloride or a mixture of two or more solvents. In one embodiment the organic solvent contains organic molecules dissolved in the solvent.

In one embodiment, this invention provides a kit for catalysis or for separation of chemicals, the kit comprising the surfaces, or the nanoparticles/structures of this invention. In one embodiment the kit comprises:
a permeable structure comprising the surfaces, or a porous structure comprising the nanoparticles of this invention;
a container comprising an inlet and an outlet such as a column, a tank, a cylinder, a pipe, a vessel, a tube in which the permeable structure or the porous structure of nanoparticles/structures is placed/packed; and
a means for introducing and dispensing a solution or a phase to and from the container.

In one embodiment, the phase is a gas phase. In one embodiment, the phase is a liquid phase. In one embodiment, the catalysis or separation kit of the invention, further comprises one or more of: reagents, solvents, a pump, a syringe, a filter, a collection chamber, a detection system, heater, cooler, gas cylinder, liquid flow controller, temperature and/or pressure gauges/controllers, valve(s), tube(s), reagent and/or solvent containers. In one embodiment, the kit can be connected to a computer. In one embodiment, the kit is compatible with automated systems. In one embodiment, the detection system is an optical detection system. In one embodiment, surfaces described herein above are surfaces of films of this invention. In one embodiment, surfaces described herein above are surfaces of structures/particles/spikes of this invention. In one embodiment, surfaces of this invention are surfaces including spikes of this invention. In one embodiment, surfaces of this invention are surfaces of films, nanostructures, structures, particles of this invention.

According to this aspect and in one embodiment, this invention provides a method for separation of chemicals/species, the method comprising:
providing the nanostructures/particles/films/surfaces of the invention;
bringing a gas phase or a liquid phase comprising at least two species into contact with the nanostructures/particles/films/surfaces of the invention, such that at least one species binds to said nanostructures/particles/films/surfaces, while at least one other species does not bind to said nanostructures/particles/films/surfaces;
removing the phase containing the unbound species.

### Catalysis

In one embodiment, this invention provides a method of reaction catalysis, the method comprising:
contacting a reactant with a surface comprising nanospikes or nano-protrusions of this invention;
   wherein said contacting results in catalysis of a reaction involving the reactant.

In one embodiment, the surface comprising the nanospikes or nano-protrusions is selected from the surface of a particle, the surface of a nanostructure, the surface of a film. In one embodiment, the nanostructure is attached to a substrate. In one embodiment, the reaction is a chemical reaction. In one embodiment, reaction catalysis is the catalysis of the reaction.

In one embodiment, the reaction rate of the reaction that is catalyzed by a surface or a particle or a structure or a film is 2-4 times faster than the rate of a corresponding reaction that is catalyzed by a similar surface that does not comprise the spikes. In one embodiment, the reaction rate of the reaction that is catalyzed by a surface or a particle or a structure or a film is 5%, 10%, 20%, 50%, 75%, 100% 200%, 300%, 500% faster than the rate of a corresponding reaction that is catalyzed by a similar surface that does not comprise the spikes. In one embodiment, the reaction rate of the reaction that is catalyzed by a surface or a particle or a structure or a film is 5%-10%, 5%-20%, 5%-50%, 5%-75%, 5%-100%, 5%-200%, 5%-300%, 5%-500% faster than the rate of a corresponding reaction that is catalyzed by a similar surface that does not comprise the spikes. In one embodiment, the reaction rate of the reaction that is catalyzed by a surface or a particle or a structure or a film is at least 5%, at least 10%, at least 20%, at least 50%, at least 75%, at least 100% at least 200%, at least 300%, or at least 500% faster than the rate of a corresponding reaction that is catalyzed by a similar surface that does not comprise the spikes.

In one embodiment, the term “catalysis” refers to the process in which the rate of a chemical reaction or a biological process is increased by a catalyst. In one embodiment, a catalyst is an agent that increases the rate of a chemical reaction. In one embodiment, a catalyst is an atom, a molecule, an ion, a radical, a surface, an aggregate of molecules, a monolayer, a multilayer, a cluster of atoms, a particle, a cluster of particles, a nanoparticle, a microparticle, a polymer, a dendrimer, a macromolecule, a biomolecule, a protein, an enzyme, a ribozyme or any other substance that increases the rate of a chemical reaction.

In one embodiment, the catalyst increases the rate of a chemical reaction. In one embodiment, the catalyst reduces the activation energy of a reaction. In one embodiment, by reducing the activation energy of the reaction, a much greater fraction of the collisions between reacting species is effective. In one embodiment, effective collision is a collision involving a reactant molecule that leads to a reaction and to the formation of products.

In one embodiment, catalysis enables to increase a reaction rate without the need to increase the temperature of the reaction. In one embodiment, a catalyst (such as the structures/surfaces of this invention) acts on the reactant species. In one embodiment, the catalyst weakens bonds within a reactant molecule. In one embodiment, the catalyst breaks bonds within a reacting molecule. In one embodiment, a catalyst arranges the reactant in a configuration that facilitates a reaction. In one embodiment, the catalyst activates the reactant. In one embodiment, the catalyst helps in the migration of the reactants. In one embodiment, the catalysts bring reactants to a close proximity. In one embodiment, the catalyst binds to one or more of the reactants. In one embodiment, the catalyst changes the polarity of the reactant. In one embodiment, the catalyst changes the electron configuration of the reactant. In one embodiment, the catalyst changes the energy state of the reactant.

In one embodiment catalysts of the present invention increase the reaction rate by reducing steric hindrance, or by increasing the fraction of collisions with effective orientations. In one embodiment, when the catalyst is confined to a nanoscale region, it can be oriented with respect to the reactant in such a way that the reactant obtains an effective orientation toward a reaction. In one embodiment, the catalyst increases the reaction rate by increasing the catalyst's cross section available for reacting with the reactant. In one embodiment, the dimensions and the geometry of the spikes and its compatibility with molecular dimensions, results in preferred reactant orientation, orientation that increases the reaction probability.

In one embodiment, the catalyst is confined to a nanoscale region. In one embodiment, a catalyst that is confined in a nanoscale domain regulates the way the reactant molecules are presented to the catalyst. In one embodiment, a catalyst that is confined in a nanoscale domain improves the way the reactant molecules are presented to, or are attached to the catalyst. In one embodiment, the spike structure regulates the way the reactant molecules approach the catalyst's surface. In one embodiment, a portion of the reactant binds to the catalyst, thus improving orientation of the reactant, and improving reaction rate.

In one embodiment, a catalyst generates an intermediate species. In one embodiment, the intermediate species is a molecule. In one embodiment, the intermediate species is a molecule adsorbed or bound to a surface. In one embodiment, the intermediate further reacts to yield another intermediate. In one embodiment, the intermediate further reacts to form a product of the reaction. In one embodiment, a catalyst is a substance that speeds up the reaction without being consumed.

### Sensors

In one embodiment, substrates of this invention are used as sensors. In one embodiment, the term ‘substrate’ refers to the substrate comprising an array of nanostructures of this invention. In one embodiment the ‘substrate’ refers to a film comprising nano-protrusions of this invention. In one embodiment the ‘substrate’ refers to a film to which nano-protrusions of this invention are attached. In one embodiment, the substrate refers to the substrate on which nanostructures or nano-protrusions of this invention are produced. In one embodiment, the substrate refers to the substrate to which nanostructures or nano-protrusions of this invention are attached. In one embodiment, the meaning of the term ‘substrate’ is apparent from the context as described herein. In one embodiment, substrates of this invention are useful as sensors. In one embodiment, substrates including nanostructures, nano-protrusions, films, particles of this invention are useful for detection of biological material and/or detection of chemicals. In one embodiment, substrates of this invention are useful for biomolecules detection, for detection of chemical compounds and other components (e.g. biological moieties, chemical species, molecules/atoms/ions). In one embodiment, sensing/detection of chemical or biological materials by substrates of this invention is applicable to materials that are dissolved/dispersed in solution. In one embodiment, sensing/detection of chemical or biological materials by substrates of this invention is applicable to materials that appear in solid state or in the gas phase or in a transient state (e.g. from solid to gas phase). In one embodiment, sensing/detection of chemical or biological materials by substrates of this invention is applicable to liquid materials.

In one embodiment, films of this invention that comprises spiked/protrusions are utilized as gas sensors. In one embodiment, gas sensors of this invention comprise continuous metal (e.g. gold) films covered by nano-protrusions as described herein. In one embodiment, gas sensors of this invention comprise substrates covered by nanostructures of this invention. In one embodiment, sensors of this invention comprise a collection of particles of this invention. In one embodiment, sensors of this invention comprise a powder comprising a collection of particles of this invention.

### Separation Techniques

In one embodiment, particles, films, substrates, nanostructures, arrays and powders of this invention are used as a separating medium for chemical/biological materials/species. According to this aspect and in one embodiment, at least one chemical/biological material that is present in a mixture or in solution is attached to the nano-structures/particles/films of this invention and is thereby separated from other components of the mixture/solution. In one embodiment, chemical/biological entities that are selectively attached to nano-structures/particles/films of this invention, can be subsequently collected. According to this aspect and in one embodiment, such separation of an entity provides a purification method for that entity. According to this aspect and in one embodiment, such separation of an entity provides quantitative and/or qualitative identification/assessment of that entity. In one embodiment, once a material is separated from a mixture/solution or other medium using structures of this invention, it can be further treated, collected, washed, dried, cooled, heated, dispersed, dissolved and manipulated for characterization or for further use by methods known in the art.

In one embodiment, color change and quantitative spectra changes (LSPR shift) with respect to the adsorption of small and big proteins (by mass) on gold islands and gold nanostars of this invention was tested. In one embodiment, gold nanostructures of this invention showed ca. 2-6 times increased LSPR shift compared to gold islands with no protrusions depending on substrate structure. Statistics regarding gold islands/nanostars surface coverage and distance between structures depending on initial evaporation thickness is described herein. In one embodiment, the spikes were obtained directly on a solid-state substrate representing stable gold islands embedded into the substrate surface. In one embodiment, substrates/films/nanostructures, arrays, particles and powders of this invention can find use in Application such as sensors, optical filters, catalysis and bio-oriented applications such as plasmonic-based or supported cellular technologies, cell manipulation and cell sorting.

### Definitions

The term ‘hedgehog-like nanostructures’ refers to the nanostructures having a base and spikes as described herein. The terms ‘star-shaped nanoislands’ or ‘star-shaped nanostructures’ or ‘nanostars’ also refer to the same nanostructures as described herein. In some embodiments, ‘nanostructures’ are referred to in short as ‘structures’. Sometimes the length of at least one dimension of the structures is in the micrometer range. However, such structures may be referred to as nanostructures in view of other dimensions. In one embodiment, a nanostructure has at least one dimension larger than the nanometer range (e.g. micrometer or millimeter range) while at least one other dimension is in the nanometer range. Such structure is referred to as a nanostructure and included in embodiments of this invention. The terms nanostars, nanostructures, nanoparticles and nano-hedgehogs are interchangeable in some embodiments of this invention. In some embodiments the nanostructures are referred to as transducers. This is in view of their function as optical transducers for analysis methods such as SERS as described herein.

In one embodiment, the description of an analyte that is bound directly to the nanostructure/film, means that there is no linker or spacer between the analyte and the nanostructure. It is the affinity of the analyte to the structure/film that causes their binding in one embodiment. In one embodiment, the description of an analyte that is bound indirectly to the nanostructure/film, means that there is a linker or spacer between the analyte and the nanostructure/film. It is the affinity of the linker to both the nanostructure on one side and to the analyte on the other side that enables indirect binding of the analyte to the structure/film in embodiments of this invention. Linkers (such as molecules) that enable such indirect binding of analyte to the structure/film are described herein above and are known to the skilled artisan.

In one embodiment, chloroauric acid or tetrachloroauric acid (HAuCl₄) forms gold chloride (AuCl₃) and vice versa. Accordingly in some embodiments, chloroauric acid or tetrachloroauric acid is referred to as gold chloride and vice versa.

Embodiments that refer to gold as the metal that forms the base, the spikes or the nanostructure, the film, the particle or any combination thereof, are applicable to any noble metal or any metal alloy of the invention in some embodiments.

In one embodiment, dewetting of a thin metal film describe the conversion of the film into a collection of particles or isolated structures.

In some embodiments, μ represents the symbol μ. In some embodiments, abbreviations are as follows: IR refer to infra-red, NP refer to nanoparticle, NS refer to nanostar or to nanostructure, Au refer to gold. In some embodiments, array is referred to as layer and vice versa. In one embodiment, a process refers to a method. In some embodiments, RI is refractive Index. In some embodiments, ‘ss’ refers to single strand while ‘ds’ refers to double strand.

In one embodiment, embodiments provided herein for nanospikes is relevant to protrusions of any shape as described herein. Embodiments described herein for nanospikes that are applicable to nano-protrusions of any other form or shape are included in this invention.

In some embodiments, nanospikes and spikes are interchangeable terms. In one embodiment, protrusions and nano-protrusions are interchangeable terms. In one embodiment, spikes or nanospikes are referred to as protrusions or nano-protrusions and vice versa.

Some embodiments disclosed herein for systems of the invention are also applicable and are included as embodiments that refer to methods of the invention that are related to or based on the described systems.

In one embodiment, a continuous is a film that is uninterrupted by areas that do not contain the film. In one embodiment, a continuous film is a film that contains voids, however, it is a film with a continuous area that surrounds or is adjacent to the voids. In one embodiment, a continuous film is a film where each film point is connected to another film point through a path that travels in/on the film. In one embodiment, a continuous film is a film that covers at least 80%, at least 90%, at least 95%, at least 99%, at least 99.9% of the surface of the substrate underneath the film. In one embodiment, a continuous film has at least one lateral dimension that has a length of at least 100 micron, or at least 1 mm, or at least 1 cm, or at least 10 cm.

Some embodiments that are described herein with reference to metal islands (with or without protrusions) apply to continuous or partially-continuous films (with or without protrusions) and vice versa. In one embodiment, silicon dioxide is referred to herein as silicon oxide as known in the art. Some embodiments that are described herein with reference to protrusions/nano-protrusions apply to spikes/nanospikes and vice versa. In one embodiment, silicon dioxide is referred to herein as silicon oxide as known in the art.

In one embodiment, a ‘nanostructure’ comprises at least one dimension in the nanoscale (1 nm-1000 nm or 1 nm-100 nm) and at least one other dimension in the micron scale or in the millimeter scale. According to this aspect and in one embodiment, such structure is referred to as a “nanostructure” for simplicity. In some embodiments, such structure is a nano-micro structure. Embodiments described herein for a “nanostructure” include structures comprising both nanoscale dimension(s) and micron/millimeter dimension(s). Some embodiments described herein for a “nanostructure” include structures with nanoscale dimension(s). Some embodiments described herein for a “nanostructure” include structures with nanoscale dimension(s) that do not have any larger (e.g. micron/millimeter) dimensions. In one embodiment, nanoparticle is referred to as nanostructure.

In one embodiment, the nano-protrusions are formed on metal islands/films/particles that were provided ready for use. According to this aspect and in one embodiment, no metal vapor deposition step is required in methods of preparations of this invention. Embodiments described herein for nanostructures/particles and films that were prepared using a step of metal vapor deposition to form the base, are also applicable and included in embodiments where the base is provided ready for use.

In one embodiment, an isolated nanostructure is a nanostructure that is not in contact with other nanostructures. In one embodiment, an isolated nanostructure is a nanostructure that is not in contact with adjacent nanostructures. In one embodiment, the space between an isolated nanostructure and neighboring nanostructure(s) does not comprise the metal or the alloy from which the nanostructure is made. In one embodiment, spacing between nanostructures are in the ranges described herein above. In one embodiment, spacing between nanostructures are in the nanometer range. In one embodiment, spacing between nanostructures are in the nanometer and/or the micrometer range.

In one embodiment, the term “a” or “one” or “an” refers to at least one. In one embodiment the phrase “two or more” may be of any denomination, which will suit a particular purpose. In one embodiment, “about” or “approximately” may comprise a deviance from the indicated term of +1%, or in some embodiments, −1%, or in some embodiments, ±1.0%, or in some embodiments, ±2.5%, or in some embodiments, ±5%, or in some embodiments, ±7.5%, or in some embodiments, ±10%, or in some embodiments, ±15%, or in some embodiments, ±20%, or in some embodiments, ±25%.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### EXAMPLES

### Example 1

### Preparation and Characterization of Nanostars

### Experimental Procedures

### Materials

Microscope borosilicate glass D263T no. 3, 22×22 (24×24) mm², with T_{g}˜557° C., (ORSAtec GmbH or Menzel-Glazer, Schott A G, Germany) glass cover-slides were used as substrates for growth after they were cut to 22×9 or 24×9 mm². The following chemicals and materials were used as purchased, hydrogen peroxide (30%, Bio-Lab Ltd., Israel), sulfuric acid (95-98%, AR-p, Bio-Lab Ltd., Israel), ethanol (abs, Gadot, Israel). Gold (99.999%, Kurt Lesker, UK); gold (III) chloride trihydrate (HAuCl₄: 3H₂O, ≥99.9% trace metals basis, Sigma-Aldrich, USA), HEPES buffer (1M, Biological Industries, Israel), Rhodamine 6G (99%, Acros Organics, China/Holland-Moran, Israel), Riboflavin (98%, Acros Organics, China/Holland-Moran, Israel). Where applicable, triple distilled water (TDW) was used (Milli-Q).

### Fabrication of Au Islands on Substrates

Glass slides were cleaned in a beaker with a Teflon glass holder containing freshly prepared “Piranha” solution (H₂O₂-H₂SO₄, 1:3 by volume) for 1 h. Subsequently, the slides were washed three times with deionized water, three times with TDW, and finally with ethanol (abs). Cleaning procedures are accompanied by the slides sonication for 5 min in an ultrasonic bath DU-32 (Argo-Lab, Italy). After the cleaning procedure, the batch of ca. 24 substrates was thoroughly dried under an N₂ stream, and then mounted to the evaporator holder. The chamber was evacuated to a pressure of 1-4×10-7 torr, and thin Au films were deposited onto the samples at a deposition rate of ˜0.1 Å s⁻¹, to nominal thicknesses of 1, 3, 5, and 7 nm (the nominal thickness is the reading of the evaporator QCM thickness monitor, i.e., the film mass thickness), using ODEM evaporator, evaporation height was 300 mm. The QCM sensor has been checked for calibration before evaporation by means of formation of 30 nm gold film on a silicon wafer surface and the step height was measured with atomic force microscopy, gravimetrical analysis or TEM cross-section. The slides were next annealed for 10 h at 580° C. in a Ney Vulcan 3-550 furnace, at a 5° C. min-1heating rate (total time required to reach 580° C. from room temperature and to anneal the samples is ˜11 h 50 min), and then subsequently slowly cooled to a room temperature inside the furnace by natural convection (ca. 12-14 h). The formed Au island films were collected and used as a primary structure for the further growth of spiked nanostructures. Prepared slides were stored in a desiccator under vacuum with relative humidity<30%.

### Formation of Gold Nanostars on Gold Nanoisland Array

Au nanostars were grown using the Au islands as a seeding layer by reduction of tetrachloroauric acid in HEPES buffer. Since the surface coverage of the annealed Au islands on the glass substrates varies with the deposited nominal mass thickness, several preparation procedures have been tested to optimize nanostar morphology and inter-star distance. In addition, other HEPES-like Good's buffer could be used (MOPC, EPPS etc.) as well as application of other solution growing techniques, including utilization of silver salts and other reducing agents.

Preparation procedure (i) The samples with Au islands were placed into a 2 mL Eppendorf test tube. In a separate test tube, 3 mL of 40 mM HEPES buffer was mixed with 30 μL of 20 mM aqueous solution of tetrachloroauric acid, mixed by vigorous shaking, and 2 ml of the resulting solution was immediately added to the test tube with Au islands slide. The test tube was then closed and immediately placed into RotoFlex Plus Tube Rotator (Argos Technologies) at 40 RPM for 30 min with the face side of the slide perpendicular to the rotor radius normal.

Preparation procedure (ii) The Au-island slides were placed in a holder in a 4 mL glass beaker with a magnetic stirrer. Next, 3 mL of 140 mM HEPES buffer was added. The stirring rate was set to 700 RPM throughout the experiment. Then, 15 μL of 40 mM aqueous solution of tetrachloroauric acid was added to the solution and the mixture was allowed to proceed stirring at 700 RPM for 30 min at room temperature of 23° C. After 30 min, the samples were rinsed gently with TDW, ethanol and dried with N₂ stream.

Preparation procedure (iii) A solution was prepared to contain 3 mL of 140 mM HEPES buffer and 15 μL of 40 mM solution of tetrachloroauric acid. Immediately after preparation, a drop of 150 AL of the solution was placed on an Au nanoisland slide. The reaction was propagated in a disposable petri dish for 30 min at 23° C. To keep the humidity constant, another small petri dish with distilled water (DW) was placed inside the petri dish with the sample. Prepared slides were stored in a desiccator with relative humidity<30% and/or under vacuum. The slides can also be stored in ethanol (abs.).

### Analyte Adsorption

Before adsorption of the analyte, in case of the gold islands slides, the slides were preliminarily cleaned with UV/Ozone generator (UVOCS Inc. model T10*10/OES/E, USA) for 20 min and EtOH (abs.) for 20 min. In case of spiked gold, after spikes growth, the slides were rinsed with distilled water, than with ethanol (abs.) or isopropanol and dried. After transducers preparation, a 1 mM solution of Rhodamine 6G (Sigma-Aldrich, USA) was prepared in TDW and was spread onto the slides in a volume of 150 μL. In case of Riboflavin, the saturated solution was prepared in TDW and the undissolved fraction was separated by centrifugation at 600 RPM for 10 min. The supernatant was separated and further used. The adsorption of both dyes was done for 1 h in a closed Petri dish with a vial of TDW or moist paper to maintain constant humidity. After adsorption, the slide was gently cleaned with TDW and dried under a nitrogen stream. Prepared slides, either with or without adsorbate molecule (analyte) were stored in a desiccator under vacuum with relative humidity<30%.

### Patterning

Patterning was performed using shadow masks for defining the areas where initial gold evaporation occurred. Following gold evaporation in the patterned areas, nanostars were formed according to the methods described herein above. The shadow masks used for evaporation are made out of SU8 (Kayaku-Microchem, USA) photoresist with a thickness range 150-300 μm. Application of shadow masks during the gold evaporation process and followed by spikes formation allowed case and preparation of different patterns for a variety of technological purposes. Shadow mask was usually placed on a substrate and sticked to it with Capton (or with Scotch tape) or it was assembled in a specially designed holder providing good hard contact between the mask and the substrate. In some embodiments, patterning provided formation of an ordered array of well-like or spot-like structures with different size applicable for e.g. ELISA-like substrates for multiple parallel and potentially automated analysis. Patterning allows integration of gold nanostar-based substrates with other technological solutions like PDMS-based structures, usually used for microfluidic device manufacturing. In order to provide high flexibility of the spiked gold structures production and device production and to fit to the specific purposes and due to the relatively high cost of stainless steel-made shadow masks available in the market, the low cost, facile fast method of shadow masks production has been used in embodiments of this invention. The method for producing the masks is based on a formation of thick layers (e.g. 150-250 μm) of SU8 photoresist in combination with convenient lithography on a Si wafer with sacrificial layer of evaporated gold (50-100 nm) or chromium. After exposure, bake and development of SU8 photoresist, the sacrificial layer can be easily removed with KCN, NaCN or gold etchant (for sacrificial layer of gold) or chromium etchant (for sacrificial layer of chromium) leading to detachment of the formed SU8-based shadow masks from the Si wafer. Time required to produce a set of ca. 24 different shadow masks with the size of 12×12 mm on a 4-inch wafer did not exceed 5 hours. The thickness of the shadow masks corresponds to the initial layer of the photoresist, and depending on the shadow mask type, it can be adjusted. Usually, in experiments described herein, the thickness was ranging between 150-300 μm. In case big patterns are required to prepare, masks can be manufactured via 3D printing, milling in metal frame and other methods as known in the art.

### Characterization

UV-vis Spectroscopy Measurements

Extinction spectra at normal incidence were measured using Cary 300 Bio (Agilent-Varian, USA) spectrophotometer in a special holder, while air was used as the baseline. Transmission spectra were recorded in the range of 370-900 nm with a scan rate, of 120 nm min⁻¹(average acquisition time per point of 0.5 s).

### Fluorescence Spectroscopy Measurements

Steady-state fluorescence measurements were carried out using a HORIBA Jobin Yvon Fluorolog-3 (Varian-Agilent, USA-Australia) spectrofluorometer, at 1 nm resolution, 0.2 s averaging time, and 5 nm excitation, emission band-pass, and detector voltage 950 V. The samples were placed on a solid sample holder, with the incident light hitting the sample at 30° from normal, and the light sensor at 60° off normal to the other side (front-faced detector). The fluorescence spectra were measured at wavelength ranges of 490-700 nm and of 285-800 nm, the excitation wavelengths were 280 nm and 480 nm. Optical and fluorescence microscopy images were obtained using fluorescence microscopes with dark/bright field modes, and phase contrast setups Micromed LUM-3 with digital camera ToupCam 5.0 MP CCD and Olympus BX-61 equipped with QImaging MicroPublisher 3.3 digital camera. To estimate the size of the clusters, ImageJ 1.6 software was used where possible. Side UV illumination was carried out using a UV lamp under 365 nm exposure wavelength.

### Surface-Enhanced Raman Spectroscopy (SERS) Measurements

Surface-enhanced Raman spectroscopy analysis of the substrates was performed on a LabRAM Raman microscope (Horiba, Japan) with lasers of 532, 633 and 780 nm, having a spot size of ˜0.2-10 μm2 (assuming spot as a circle with a diameter of ˜0.5-3.6 μm).

### Circular Dichroism (CD) Measurements

The CD spectra were used to monitor the conformational transformation of Riboflavin on substrates. The CD spectra were recorded with a Chirascan™ V100 Circular Dichroism Spectrometer (Applied Photophysics Ltd., UK) with a wavelength spectra range of 450-750 nm and a 1.2 nm wavelength step for 1 second with light incidence perpendicular to the substrate faces.

### Optical and Fluorescent Microscopy

Optical and fluorescent images of the protein samples were obtained using SteREO Discovery. V12 stereo microscope and Axio Observer 7 inverted microscope (Carl Zeiss, Germany) equipped with Axiocam 503 and 506 color-and mono-HDTV cameras (Carl Zeiss, Germany). UV source in both cases was HXP 120 V Fluorescence Mercury-based Light Source (365 nm, Carl Zeiss, Germany).

### Confocal Microscopy

For confocal microscopy, samples were prepared by depositing the aqueous solutions of analytes, without further purification, onto glass slides. The protein samples were analyzed using confocal microscope LSM 800 (Carl Zeiss, Germany) with the following LED lasers: 405 nm (for violet/blue excitation) 488/564 nm (for green excitation) and 680 nm (for red excitation).

### High-resolution Scanning Electron Microscopy (HRSEM)

HRSEM images were obtained using Ultra-55 and SIGMA Ultra-high-resolution SEM (Carl Zeiss, Germany) with accelerating voltage of 3-10 kV for both In-Lens, energy selective backscattered (ESB) and Everhart-Thorney secondary electron (SE2) detectors under vacuum of <2-5·10⁵ mbar with working distance of 3-6 mm. The slides were placed onto Al stubs and fixed with carbon tape and partially coated with carbon paste and dried for 10-20 min. Next 2-3 nm of Cr (ODEM or Yo evaporator) or Ir (Safematic CCU-010 HV high vacuum sputter coater, LabTech, UK, vacuum of <5·10⁵ mbar) were deposited before imaging to improve samples conductivity, image contrast and stability during imaging.

### Atomic Force Microscopy (AFM)

The sample was imaged on an AFM JPK Nano wizard 4 (Germany), assembled with Olympus optical microscope (Japan) for sample finding using AC240 or AC160 cantilevers in tapping mode. The images were processed with JPK data processing software.

### Transmission Electron Microscopy (TEM)

For regular TEM imaging the gold island array was transferred from the glass substrate onto a TEM grid as follows: a drop of 1% nitrocellulose (or 2% of collodion) solution was placed on the top surface of an annealed Au film and a copper grid (SPI, West Chester, USA) was carefully placed on the drop, followed by rotation of the assembly on a spin coater wheel until complete dryness (30 s, 500-100 RPM, 100 RPM/s acceleration). The glass substrate with the mounted Cu grid was then floated on 5% HF solution (or buffered oxide etch solution: 6:1 volume ratio of 40% NH₄F in water to 49% HF in water) to completely dissolve the glass. The grid with the island array was lifted, washed in distilled water and dried under a nitrogen stream. Prior to TEM measurement, the samples were covered with a thin carbon layer using an Edwards FTM6 evaporator.

### Cross-section Focused Ion Beam (FIB) Lamellae Preparation

Lamellac for cross-sectional TEM imaging were prepared using the Helios 600 dual-beam microscope (ThermoFisher Scientific, Waltham, MA USA). Samples were precoated with a carbon layer by thermal evaporation process (Safematic CCU-010 HV high vacuum sputter coater, LabTech, UK). Region of interest (ROI) for the lamellae preparation was locally protected by double CVD layer: (1) e-beam induced carbon layer of about 0.5 μm, (2) ion beam induced Pt layer of ˜1.5 μm. The final thickness of the lamellae was estimated as 50-100 nm. TEM images were acquired using the Titan THEMIS transmission electron microscope operating at 200 keV. equipped with a charge-coupled device (CCD) camera (2 k, Gatan Ultrascan 1000). Selected-area electron diffraction (SAED) was performed using a 50 nm aperture.

### Numerical Simulation

Electromagnetic numerical calculations were performed using a COMSOL Multiphysics software package and were based on a finite element method. It is assumed that Au NPs present oblate spheroids with a diameter of 30 nm and 50 nm and a height of 20 nm. A hexagonally packed Au NP layer with a center-to-center distance of 100 nm was used in the calculations; the surface coverage of the Au NPs in such an arrangement is 23% and fits experimentally found values. In the initial configuration, the Au NP layer was placed above the substrate with a separation of 2 nm from the substrate surface to avoid singularity in the calculation. For Au and glass, dispersive dielectric constants from the literature were used.

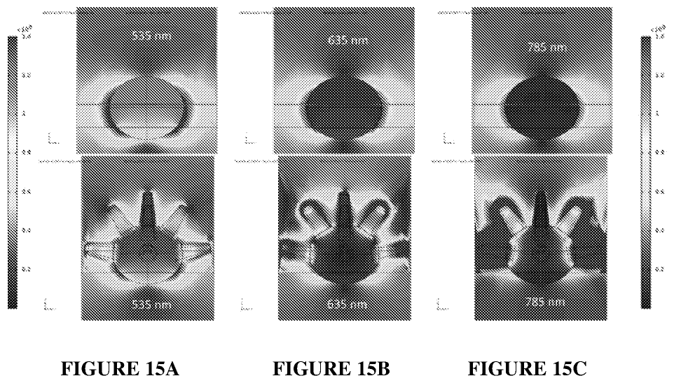

The color scale bar in all the simulation images (see for example FIG. 15) is in V/m and shows normalized local electric field intensity distribution (or strength) around a nano-object upon interaction with evanescent (or incident) electromagnetic wave of certain wavelength at the slice, passing through the center of the nano-object (XZ slice in all cases). When electromagnetic wave frequency is becoming closer to the natural plasmon frequency, the resonance state approaches. This has close dependency on nanoparticle size, shape and distance.

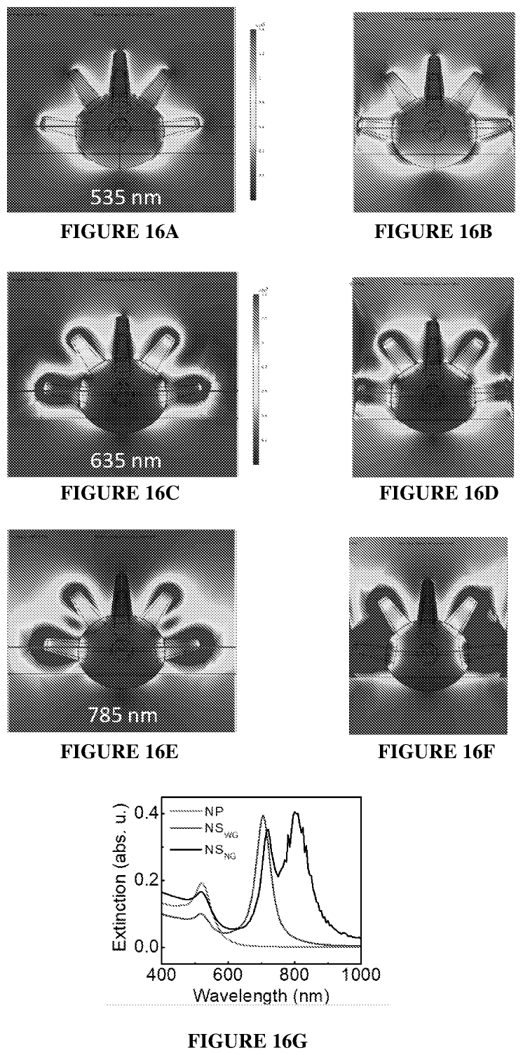

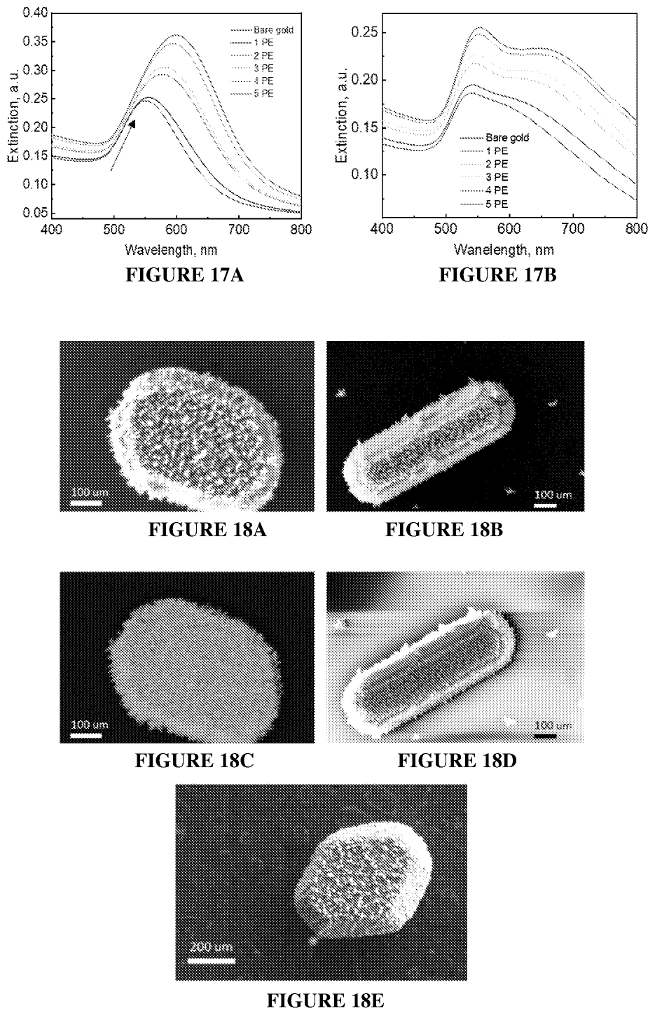

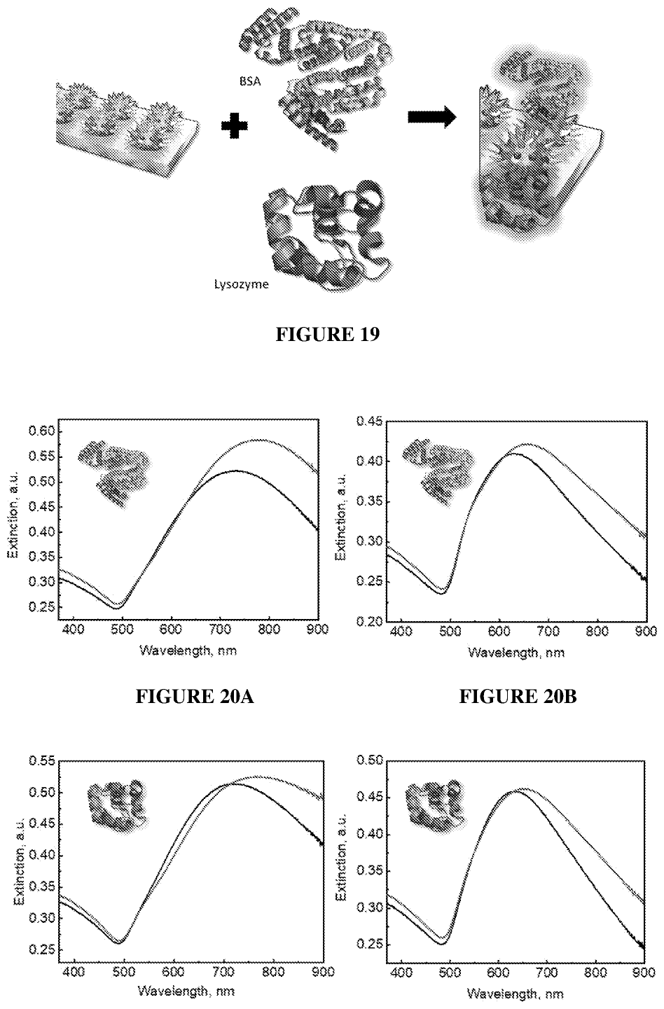

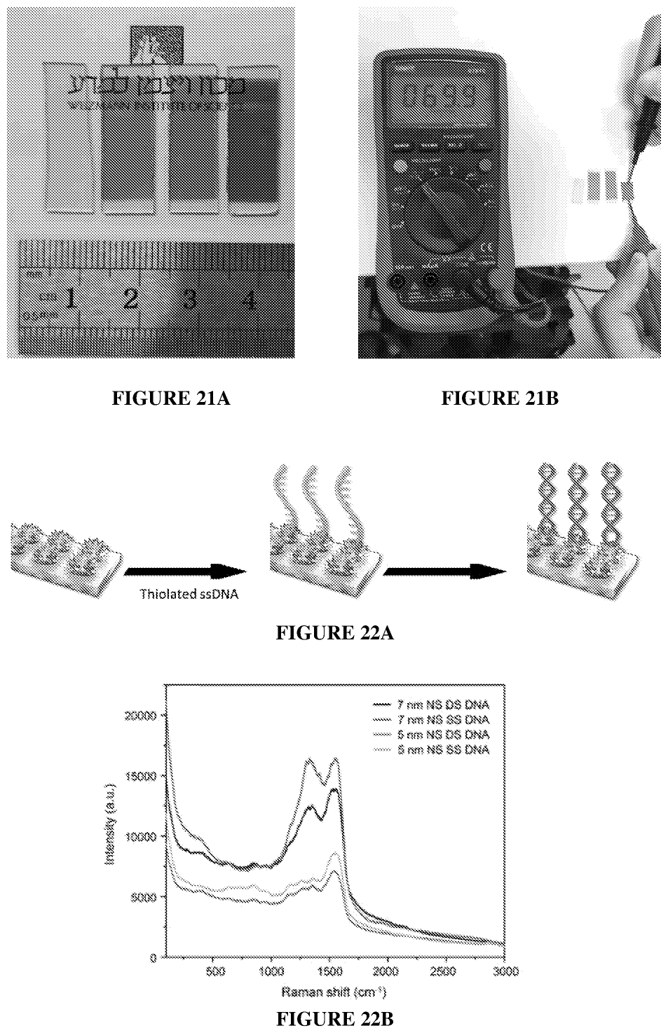

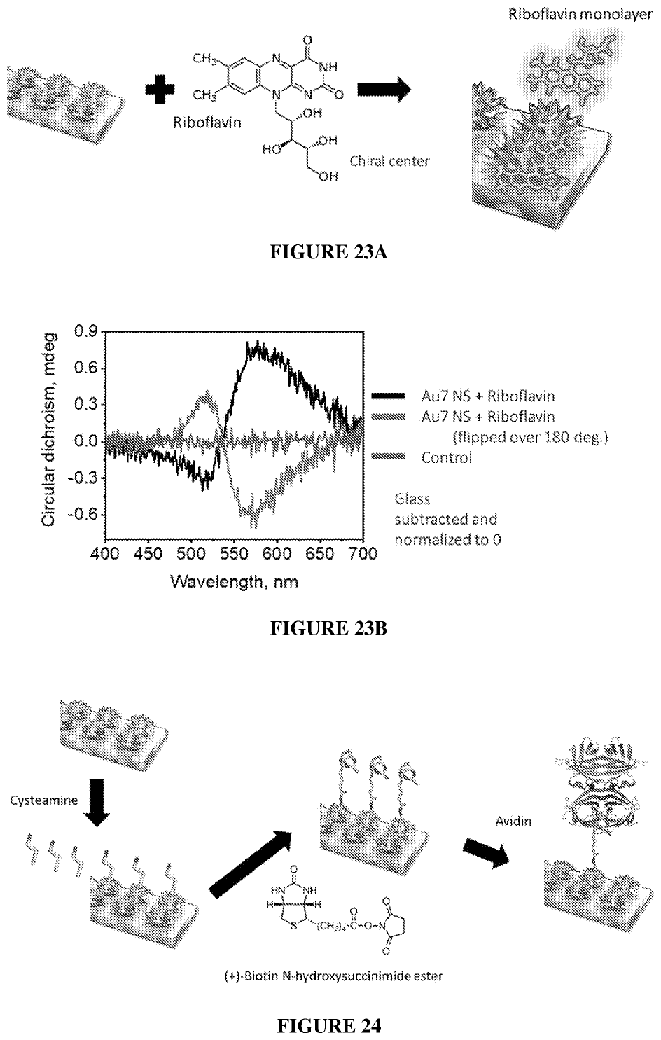

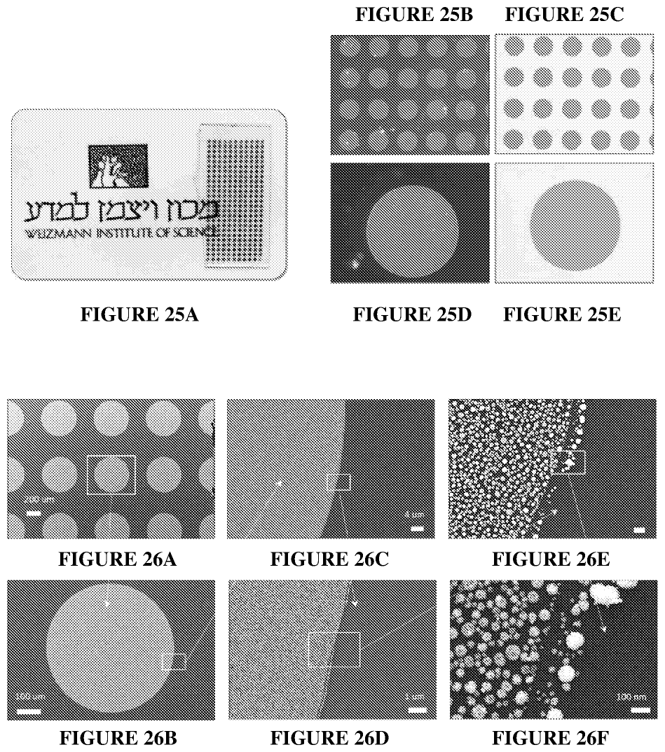

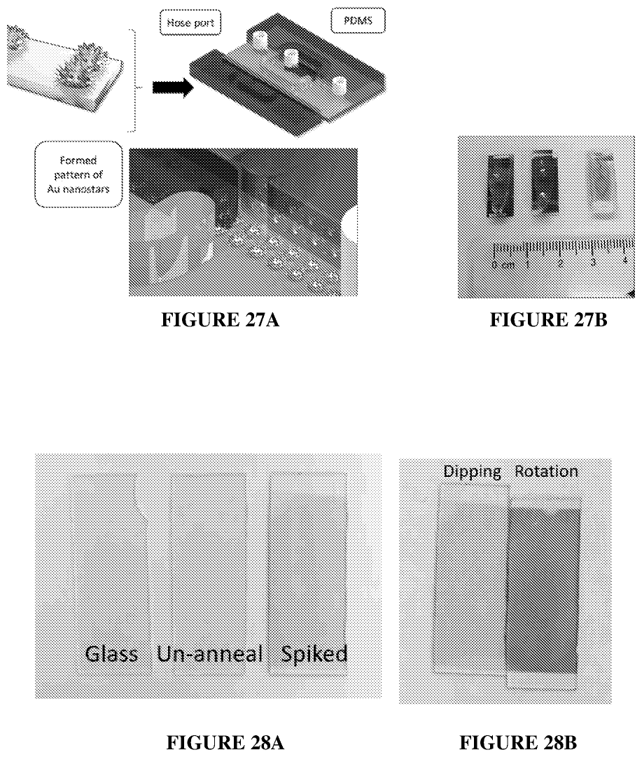

In FIG. 16, a ‘real size’ nanoparticle from the model of simulation window is shown. Specifically, in the model presented in the figures a narrow gap of 5 nm was used while wide gap was 20 nm. In addition, a large number of additional simulations varying other different parameters have been performed. In the model presented here, a single cell element is chosen, representing nanoparticle with spikes partially embedded in “glass”. In simulation the periodicity of this element can be set to repeat with certain gap between “spikes” (not geometrical centers of the structure). Here a virtual array of 100 elements (10*10) have been used.

### Aspect Ratio (AR)

Aspect ratio (AR), see FIG. 3B, and FIG. 41C was automatically calculated from ImageJ (1.5 image analysis software). AR is a measure of the ellipticity of the structures. The more elliptical the structures, the higher is the aspect ratio (major axis of the structure is in the numerator while minor axis is in the denominator). Ellipses with aspect ratio of 1:1 is a circle.

### Results and Discussion

FIG. 1A and FIG. 1B schematically present the procedure to form Au nanostars (Au NSs), discontinuous films on glass substrates. Initially, ultrathin discontinuous (according to nominal mass thickness quartz crystal microbalance monitor) Au unannealed films were obtained by e-beam or resistive evaporation of Au on borosilicate glass coverslips with RMS roughness of ˜1 nm. The nominal mass thicknesses used in this research were 1, 3, 5, and 7 nm. At these thicknesses, the as-deposited Au films have a depercolated (island-like) structure, and also possess low stability (FIG. 2B-FIG. 2E), while the percolation threshold for Au films on glass is approximately 8 nm (the limit when continuous gold film starts to form). The percolation threshold is highly important as dewetting/coalescence processes vary substantially at percolated films forming much larger islands.

The as-deposited Au islands have complex structures (see examples in FIG. 2B-FIG. 2E) resulting in a very broad localized surface plasmon resonance (LSPR) band with a maximum in the near-IR region. Therefore, the as-deposited ultrathin metallic films have been exposed to high-temperature treatment at 580° C. for 10 h in an ambient atmosphere. This is in order to (i) trigger a coalescence process in the initially depercolated islands to form well-defined (e.g. round) nanoparticles in some embodiments, and (ii) stabilize the islands on glass substrates.

It is known that the adhesion of Au to glass is weak. Therefore, the formation of either a metallic layer (Ti or Cr) and their oxides, or a self-assembly monolayer of mercapto- or amino-silane has been previously developed to stabilize Au NPs on the glass. However, the deposition of such metallic films significantly reduces the optical intensity of the transducers. Moreover, in both cases (inorganic/organic precursor layers), such buffer layers are highly sensitive to thermal treatment at ambient atmosphere and cannot be used in high-temperature harsh environment. Therefore, in this example, another approach was applied to stabilize Au islands, an approach that involves the partial embedding of the islands in the glass matrix by exposure to high temperature as described herein above. This approach has several substantial advantages: (i) formed Au islands are free from any capping molecules and are ready for their further structural and surface modifications; (ii) Au islands are extremely stable in any solvent, solvent mixtures and biological buffers, and can be easily applied to advanced lithographic processes without losing structural integrity; (iii) Au islands have a single crystalline structure in some embodiments; (iv) the LSPR peak of the annealed island film is well-pronounced with a narrow full width at half maximum (FWHM); and (v) the deposition/annealing approach forms a uniform coating of Au nanoislands over large area indicating that the process is easily scalable.

Despite these aforementioned advantages, high temperature annealing results in some embodiments, in the formation of well-defined spherical-like islands with inter-island distances that are of the same order as the NP size (FIG. 2F-FIG. 2I). This morphology does not fit the SERS requirements for hot spots, i.e. highly localized regions of intense local field enhancement. The inter-island spacing achieved is too large.

There are several routes to locally enhance the electromagnetic field strength near noble metal nanoparticles by taking advantage of the plasmonic phenomenon as follows: (i) decrease interparticle spacing to the order of single nanometers; or (ii) to increase the nanoparticle surface roughness by growing sharp features. The latter structural modification is commonly called nanostars. Both approaches have been previously applied for SES sensing.

However, in all these previously-tested cases, the nanoparticle assemblies/agglomeration (i) and nanostars (ii) were synthesized in solution. Such solution-based particle synthesis suffers from several limitations: (i) the solution synthesized nanofeatures should be subsequently immobilized on a solid substrate in a stable and reproducible manner. This significantly complicates the transducer fabrication process. (ii) Solution synthesized nanoparticles commonly consist of capping agents, such as surfactants or organic acids anions (citrate, ascorbate, etc.) required to either stabilize nanoparticles in solution preventing their aggregation and precipitation or create specific surface charge on a nanoparticle, that reduces the surface area that is available for analyte binding.

In this work, in one embodiment, it was found that all these aforementioned synthetic and structural complications can be overcome when the noble metal nanoparticles are initially formed steadily on the solid substrate as described herein.

In this example, the annealed Au islands attached to a substrate were modified using AuCl₃ reduction in HEPES buffer resulting in the growth of Au spikes selectively on the Au— island surface leading to the formation of star-shaped nanoislands. Here, the initially stabilized Au nanoparticles are used as seeds in the electroless growing process of the gold spikes.

### Example 2

### Additional Results

In one embodiment, for reproducibility tests, four different slides were prepared at the same conditions, (slides were placed at the same place of the evaporator's sample holder, the evaporation parameters were exactly the same in all cases, and the spikes were grown at the exact same conditions: 30 min in 2.03 ml of growth solution at 23° C.). The resulted slides color was uniform across the slide and the spectra were similar. The slides are quite stable under reasonable mechanical stress since they are comprising solid glass.

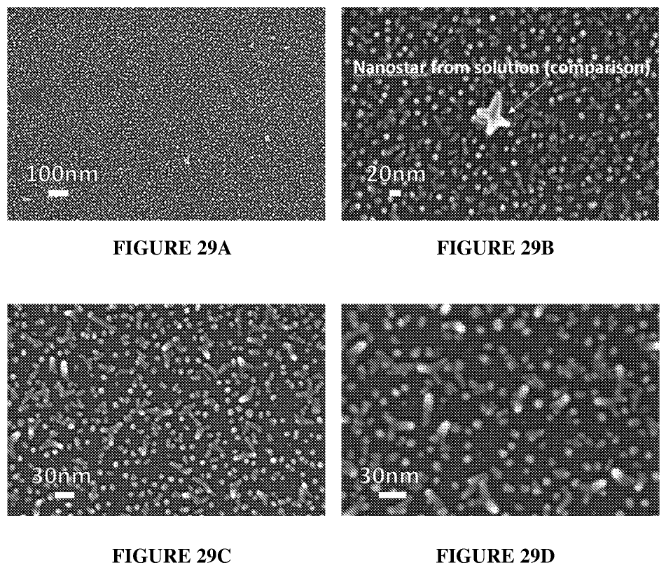

The procedure of spikes growth was extended onto extremely thin gold films beyond 1 nm nominal thickness limit (<1 nm). At these conditions extremely small gold islands can be obtained after high temperature annealing. As in the case of spikes growth at the nominal gold evaporation thickness>1 nm, this (thin) process was also split into two ways of spikes growth: a standard way using a rotator (rotation procedure) and a simple dipping of the slide into a growing solution in a beaker (dipping). Sec FIGS. 28-29: Top left-comparison of the slides transparency and color for pure glass, after 1 Å of gold evaporation thickness and after spikes formation via dipping procedure; bottom left-comparison of the slides (1 Å of gold evaporation thickness) obtained via two different procedures, dipping (left) and rotation (right); right panel-SEM images at different magnification of the gold structures with spikes grown at the slide of 1 Å of gold evaporation thickness using dipping procedure.

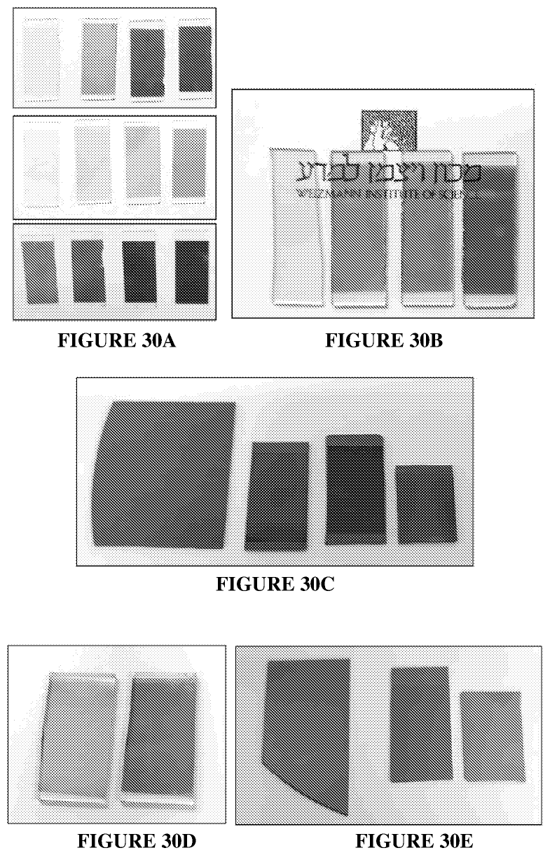

The procedure of spikes growth was not limited by borosilicate glass only, but was also conducted using other substrates such as quartz that provide transparency in the UV region, silicon wafers with or without thin or thick native or artificially grown oxide (e.g. via PECVD-plasma-enhanced chemical vapor deposition process) that provide reflectance mode, glass with FTO (fluorine doped-tin oxide) or ITO (indium-tin oxide) that provide layer conductivity. However, in these cases after evaporation and annealing, gold islands were not embedded when annealing at <600° C. since these structures represent crystal forms, not amorphous glass, and hence they do not have a glass transition temperature. The embedding of the particles can be possibly reached at temperatures close to the melting point of these substrates (depending on substrate class and its crystalline form, for quartz ca. 1650° C., for silicon ca. 1400° C.). Additionally, thin and flexible glass slides (<100 μm in thickness) can be used as optical fibers as well. Results for the various substrates can be seen in FIG. 30.

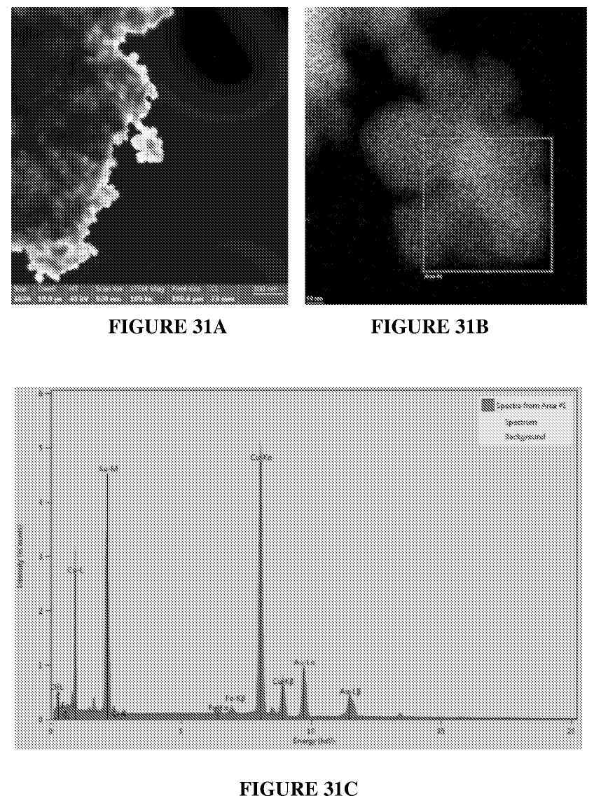

FIG. 31A and 31B show HAADF (high-angle annular dark-field scanning transmission electron microscopy) STEM images of nanostars transferred onto TEM grid at different magnifications and FIG. 31C electron energy loss spectra (EELS, right panel) showing different plasmon modes at different regions of scanning.

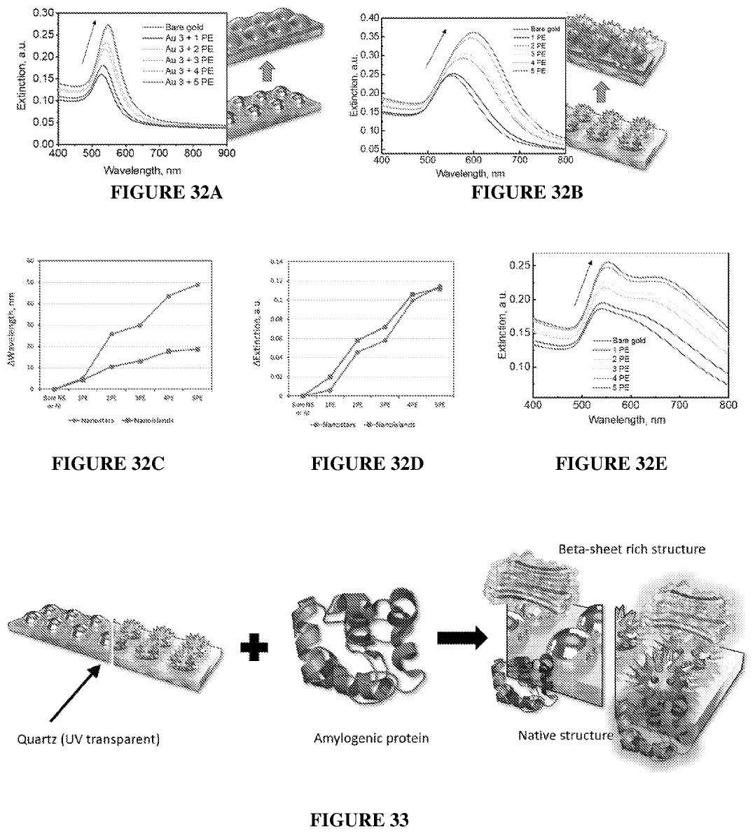

FIG. 32 shows the slides of comprising of nanoislands or nanostars were subjected to a layer-by-layer self-assembly test using organic polymer (polyelectrolyte) and the spectra were measured in terms of plasmon peak shift after each layer and compared. Slides were covered with alternating layers of positive (poly-allylamine hydrochloride) and then negative (poly-styrene sodium sulfonate) starting from positive one.

Comparison of the spectra of gold islands and nanostars after polyelectrolyte layer-by-layer self-assembly and respective relative changes of the plasmon peak position and respective relative changes of the extinction for both systems.

Examples of facet-selective types of spikes growth on big islands obtained after thick gold layer evaporation (>13-15 nm) followed by high temperature annealing. Growth either on a side of the gold island or full coverage of the island with spikes can be controlled by changing deposition parameters such as initial concentrations of gold salt or buffer, buffer type, solution temperature, deposition procedure (rotation or dipping). Gold island crystal type effects spikes growth in terms of their density on a single island (depending on the gold nominal evaporation thickness).

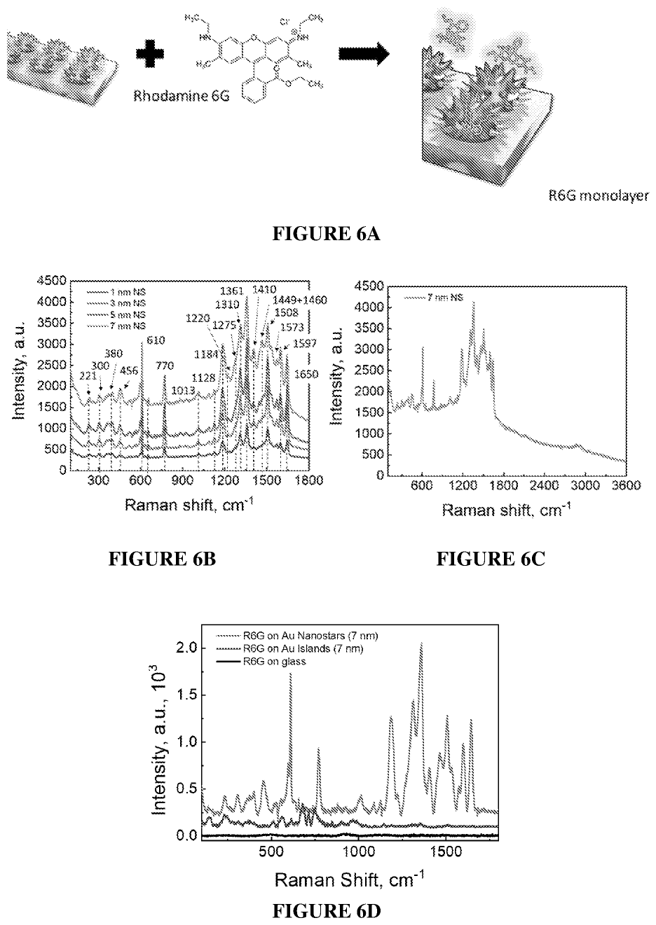

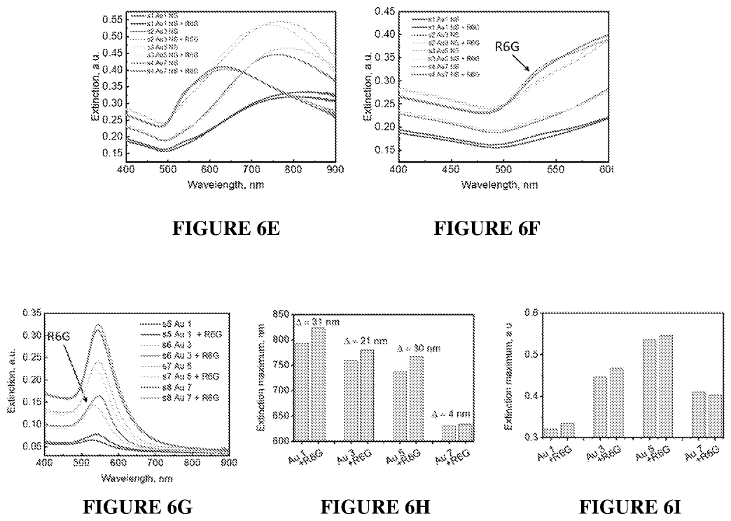

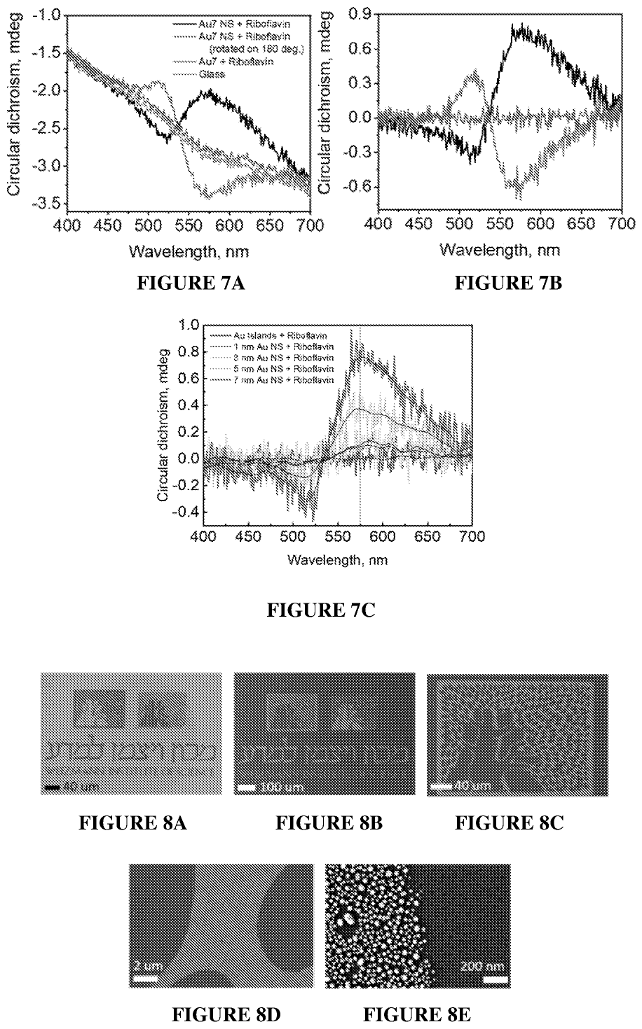

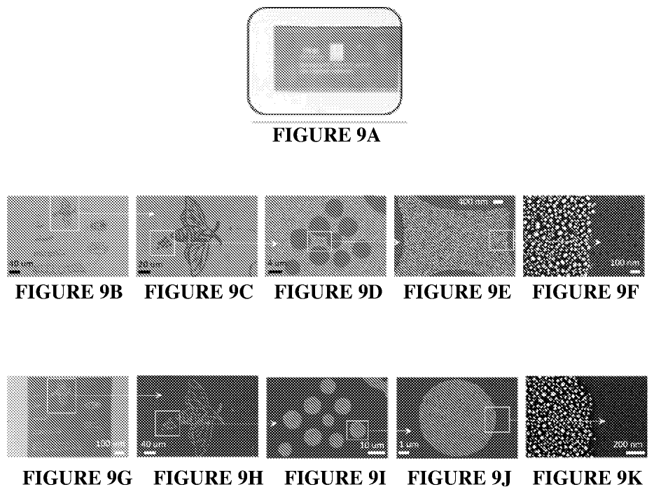

FIG. 6 shows comparative results on Rhodamine 6G (R6G, 1 mM) adsorption on gold nanoislands compared to gold nanostars. In case of gold islands there is an overlap between the R6G and plasmon peaks, while in case of nanostars they are located separately. Bottom diagrams show absolute plasmon peak and extinction maxima position before and after R6G adsorption as well as relative changes in these parameters during the adsorption process.

FIG. 33 represents the concept of IR. Raman, CD and TERS comparison study of proteins structure investigation using spiked substrate as signal of interest enhancer. The concept of the study related to revealing protein structural conformation changes when interacting with plasmonic structures located on a surface additionally serving as specific signal amplifiers.

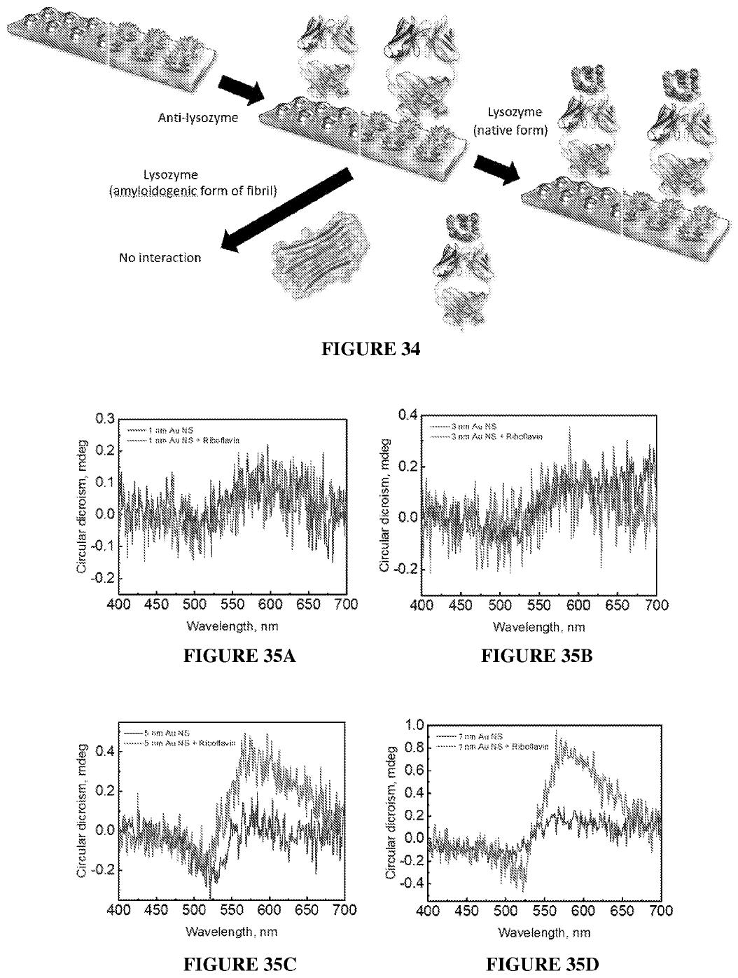

FIGS. 33 and 34 show concept schemes of comparative study of antigen-antibody interaction on surface-tethered nanostars using gold nanoislands system as comparison system.

FIG. 35 shows an example scheme of CD spectroscopy application study for surface-bound chiral molecules (example of (−)Riboflavin, adsorbed on the gold nanoislands-based and gold nanostars-based surface). CD spectra of gold nanostars slide with adsorbed riboflavin compared to pure glass and gold nanoislands of 7 nm nominal thickness with adsorbed riboflavin before (left) and after (right) of subtraction of glass baseline.

The FIG. 35 spectra of different gold nanostars substrate were obtained on corresponding gold nanoislands films of 1, 3, 5, 7 nm of nominal gold thickness evaporation after adsorption of riboflavin and before.

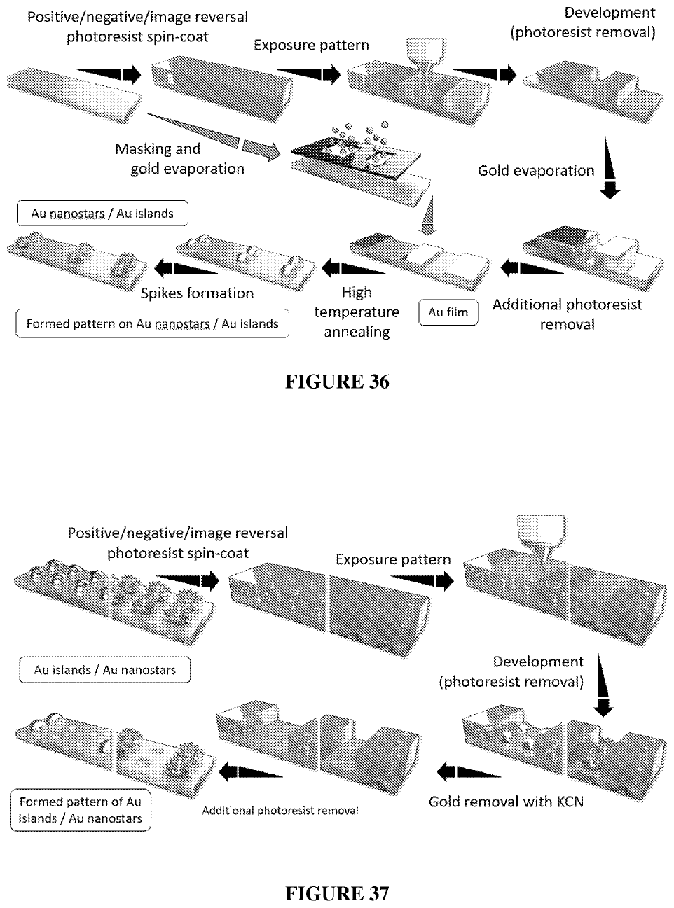

An example scheme demonstrating possibilities of patterns creating with gold nanoslands/nanostars using simple lithographical or masking steps. When choosing lithographical way, a positive/negative/image reversal photoresist was spin-coat onto glass slide followed by pattern exposure and photoresist removal (development). Next gold evaporation step was done and additional photoresist removal with a suitable solvent was carried out followed by the procedure of high temperature annealing and spikes growth. When choosing masking, a preliminary manufactured mask with the pattern was imposed onto a glass slide flowed by gold evaporation, high temperature annealing and spikes growth. As can be seen in FIG. 36.

Another scheme of patterns creation using lithography on already existing nanoislands/nanostars structures. On the first step, positive/negative/image reversal photoresist was spin-coat onto glass slide followed by pattern exposure and photoresist removal (development). Next, the gold in any form was removed with cyanide (KCN) followed by complete photoresist removal. As can be seen in FIG. 37.

There are many ways of potential integration of PDMS-based microfluidic devices with nanoislands/nanostars arrays especially in combination with patterning. Selective preparation of nanoislands/nanostars paths followed by alignment with the micro-channels allowed to free glass surface out of nanoparticles for good sticking with PDMS. In one example, a liquid containing analyte or e.g. cells may pass by the pattern in microfluidic channel and the analyte will bind to the spiky structures. The sample can be analyzed any suitable technique (SERS, CD, LSPR etc.).

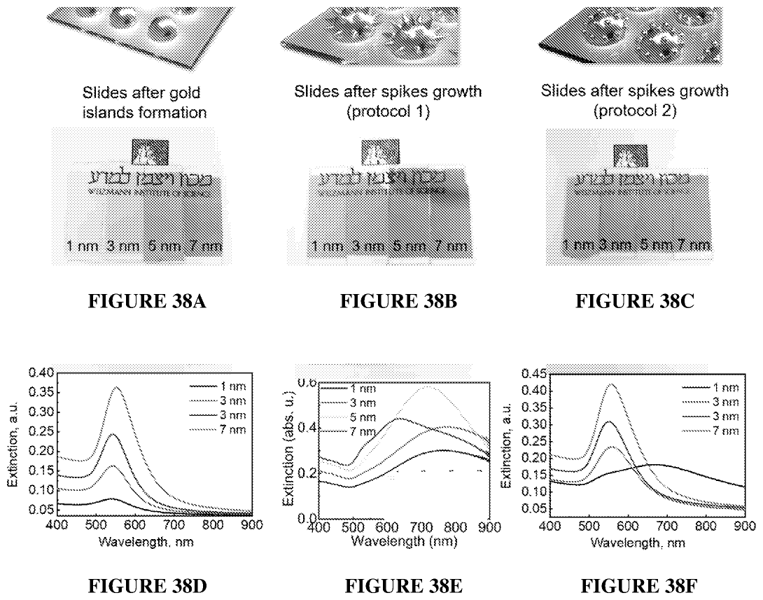

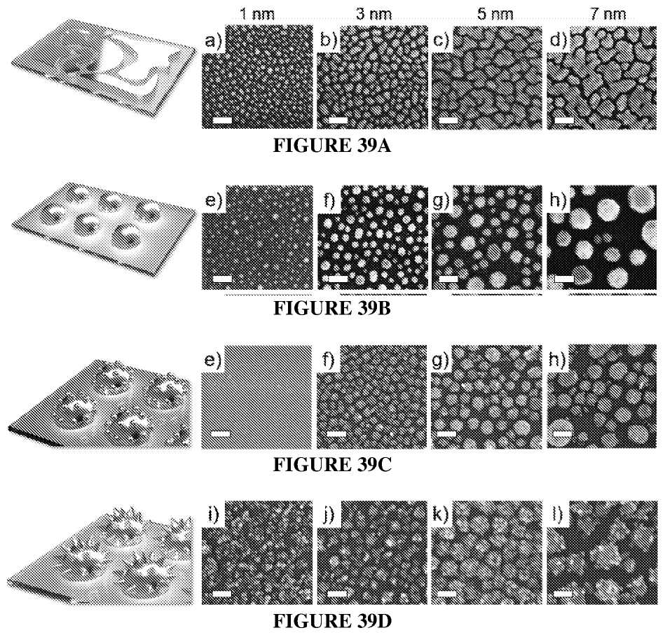

FIG. 38 shows a comparison of the gold islands slides and spiked gold structures (digital images and corresponding LSPR UV-vis spectra) obtained after 1, 3, 5, 7 nm of nominal gold thickness evaporation and high temperature annealing. Protocol 1 corresponds to the spikes formation from 2 ml solution in Eppendorf test tube prepared from mixing of 3 ml 100 mM HEPES and 30 μl 20 mM HAuCl₄ (pH is ca. 7.2-7.4) for 30 min in a rotator at 40 RPM at ca. 23° C. Protocol 2 referred to the spikes formation from 2 ml solution in Eppendorf test tube prepared from mixing of 3 ml 140 mM HEPES and 15 μl 20 mM HAuCl₄ (pH is ca. 7.2-7.4) for 30 min in a rotator at 40 RPM at ca. 23° C.

FIG. 39 shows SEM images (scale bar is 40 nm) corresponding to the slides prepared in accordance with the protocols 1 and 2 in FIG. 38.

### Reconstituted Silk Fibroin Solution (RSF) Preparation

Silk fibers spun by larvae of the silk moth Bombyx mori were degummed according to an established protocol. Briefly, silkworm cocoons were chopped and then boiled in 20 mM sodium carbonate solution (≥99.5%, Fischer Chemical, USA) at a ratio of 200 mL solution per gram of raw cocoon. The degummed fibers were then washed and dried, followed by dissolvation at 60° C. in a concentrated solution of aqueous lithium bromide (≥99%, ReagentPlus®, Sigma-Aldrich, USA; mass ratio 4:5 LiBr:H₂O) at a concentration of 100 mg/ml. The resultant solution was centrifuged and dialysed against Milli-Q water over 48 hours using a 10 kDa cut-off membrane (Snakeskin, Thermo Fisher).

Rockwood, D. N. et al. Materials fabrication from Bombyx mori silk fibroin. Nat. Protoc. 6, 1612-1631 (2011).

### Proteins Immobilization Protocols

Before adsorption of the analyte, the gold islands slides were preliminarily cleaned with UV/Ozone generator for 20 min and EtOH (abs.) for 20 min. The slides of nanostars after obtaining cleaned with isopropanol or EtOH (abs.). After transducers preparation, a drop (100-200 ul) of lysozyme or RSF solution with concentration of 0.1-10 mg/ml mM prepared in TDW or PBS (Lysozyme only) was spread onto the slides. Adsorption time 20 min-1 h. After adsorption, the slides were gently cleaned with Milli-Q water and dried under the nitrogen stream and then transmittance UV-Vis spectra were measured.

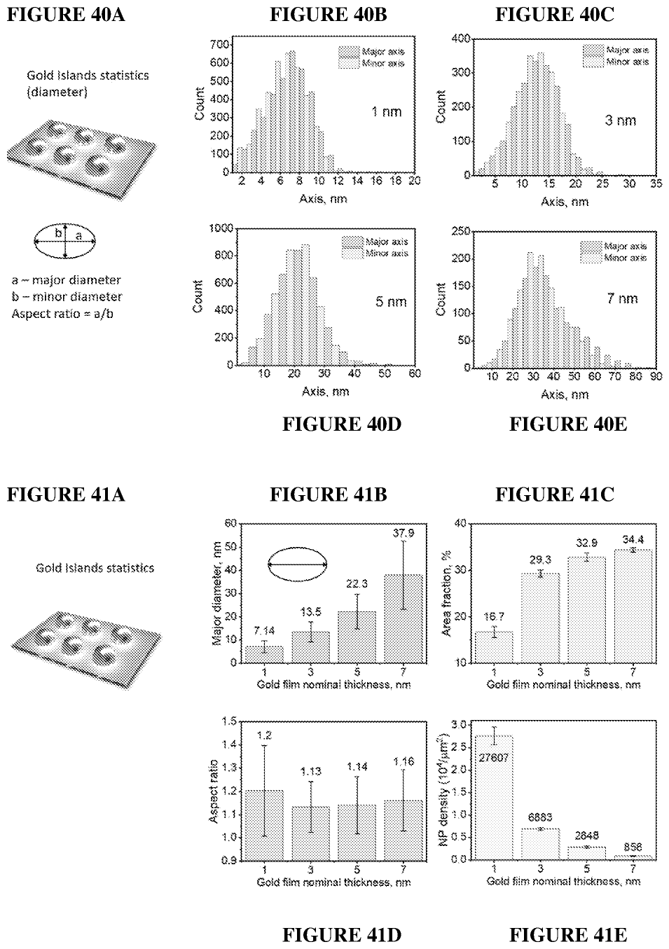

FIG. 40 shows statistical parameters (number of particles vs. minor and major diameter) distribution of gold islands with respect to the nominal gold evaporation thickness of 1, 3, 5 and 7 nm.

FIG. 41 shows a comparison of the statistical distribution parameters (major diameter, area fraction, aspect ratio and islands density) of gold islands with respect to the nominal gold evaporation thickness of 1, 3, 5 and 7 nm.

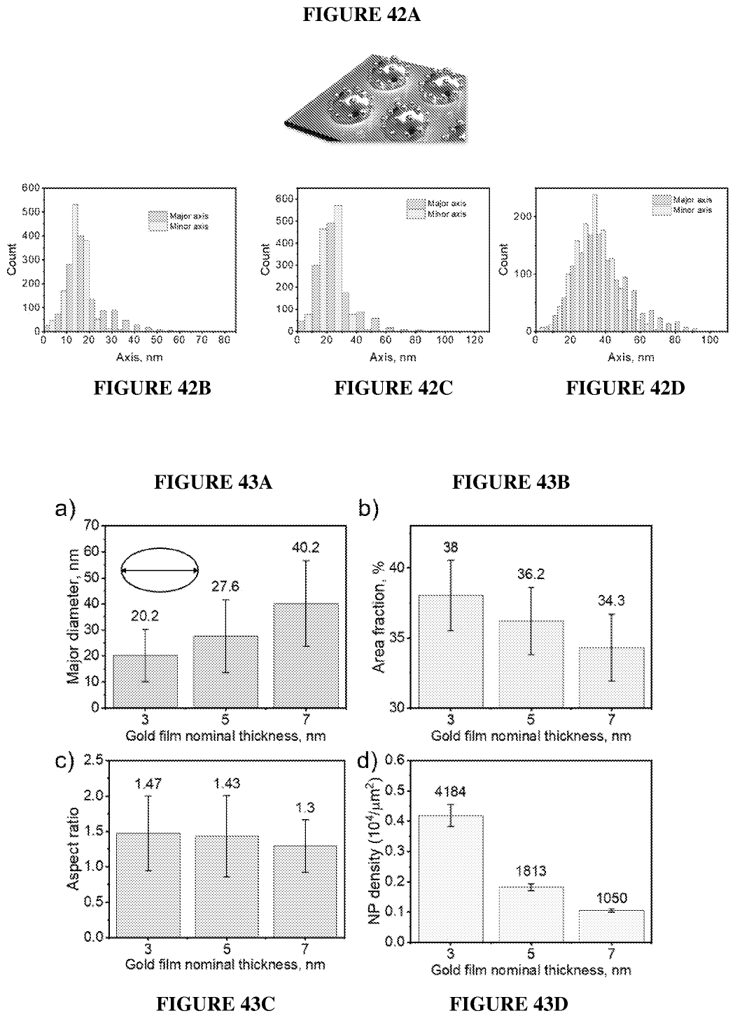

FIG. 42 shows statistical parameters (number of particles vs. minor and major diameter) distribution of gold nanostars with respect to the nominal gold evaporation thickness of 1, 3, 5 and 7 nm obtained in accordance with the protocol 2.

FIG. 43 shows a comparison of the statistical distribution parameters (major diameter, area fraction, aspect ratio and islands density) of gold nanostar with respect to the nominal gold evaporation thickness of 1, 3, 5 and 7 nm obtained in accordance with the protocol 2.

After adsorption of the lysozyme or RSF proteins and monolayers formation, the slides change their color both in case of adsorption on nanoislands (1, 3, 5 and 7 nm of gold nominal evaporation thickness), and in case of nanostars (1, 3, 5 and 7 nm of gold nominal evaporation thickness) since the plasmon of nanoparticles changes. In case of gold nanostars there is more pronounced color change that can be detected by naked eye in case of adsorption of both proteins for 3, 5 and 7 nm of gold nominal evaporation thickness compared to gold nanoislands. For RSF the color changes are more pronounced compared to lysozyme since the molecular mass of the RSF is bigger than lysozyme, hence it occupies more space in the surrounding area of nanoparticle that causes more pronounced shift of LSPR band.

LSPR spectra of gold islands/nanostars before and after either lysozyme or RSF adsorption.

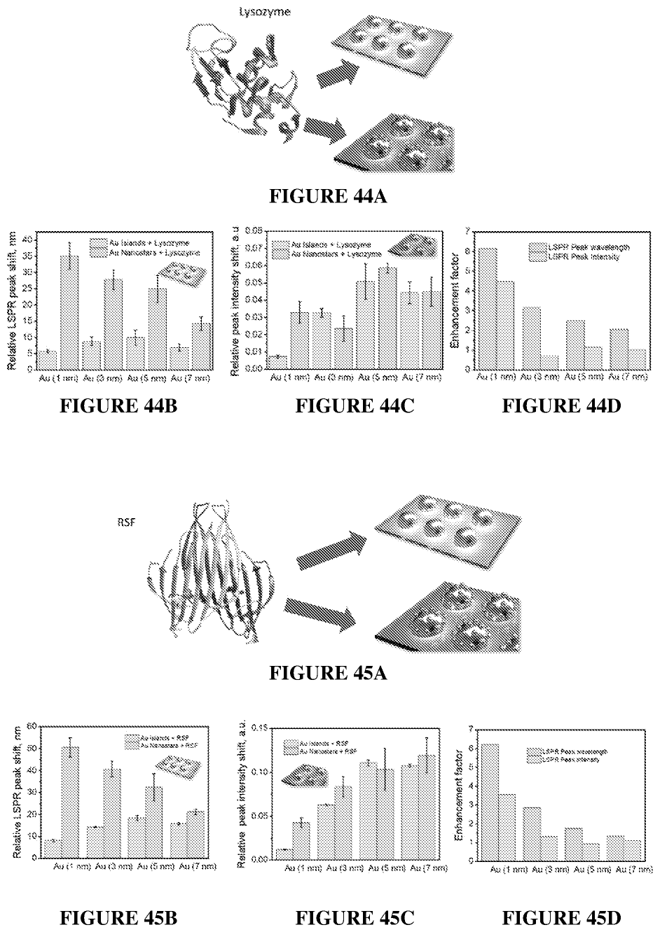

According to the spectra, in all the cases, gold nanostars show ca. 2-6 times increased LSPR wavelength shift compared to gold islands depending on substrate structure both in the case of lysozyme and in case of RSF adsorption. The more pronounced shifts of LSPR band in case of nanostars display more sensitive behavior of plasmon response with respect to analyte adsorption. This is a benefit in comparison to the spherical nanoparticles since the system sensitivity is increasing. Moreover, since the particles not dispersed in solution but located on a surface and they are the part of a slide itself, it is possible to manipulate with the spikes parameters (length, thickness, amount) as well as gap between of them to tune the system in such a way that it will allow it to be suitable for different types of applications either in different spectroscopic techniques and other industrial fields such as catalysis (since the surface density and roughness is increased), objects manipulation with plasmonic field, etc. See examples in FIGS. 44-45.

A variant of the technique is presented in which the gold nanoisland slide before spikes formation is treated with UV Ozone for 30 min and then it is placed into the spikes growing solution.

A variant of the procedure that employs surfactants to grow the spikes was presented. It was shown that Triton X family of non-ionic surfactants may initiate the formation of bridges between of gold islands and form crystalline structure with facets after 24 hours of growth in case of small islands (5 nm of nominal gold evaporation thickness), but in case of big islands (20 nm of nominal gold evaporation thickness), the application of Triton X causes formation of rather big spikes.

It was found that the spikes formation is possible not only at the common pH value of solution (7.2-7.4, that is close to HEPES pKa of 7.55) but also at higher pH values (8.5).

It was shown that application of other Good's buffers in addition to HEPES, namely (at least, maybe many other also may work!) MES, HEPPS, MOPC are also suitable systems to growth the spikes.

Application of HEPPS appears to be similar to HEPES both in case of small (5 nm of nominal gold island evaporation thickness) and big (20 nm of nominal gold island evaporation thickness) islands.

Application of MES instead of HEPES leads to formation of spherical particles on small islands (5 nm of nominal gold island evaporation thickness) instead of noticeable spikes but in case of big (20 nm of nominal gold island evaporation thickness) islands it causes formation of spikes on a 111-facets and additionally spherical particles in the space between the 111 facets.

In case of MOPC it is possible to obtain an extremely fine facet-selective uniform surface coverage of the big gold island (20 nm of nominal gold island evaporation thickness) with the spikes. Such kind of islands are suitable candidates or tip-enhanced spectroscopies.

Moreover, the method of nanostars transferring onto grid with acetyl cellulose followed by glass dissolution in hydrofluoric acid (HF) was also applicable as a method of nanostars transferring from solid glass template onto any other material that is stable for HF treatment (flexible or solid, organic or inorganic) that was drop-casted and dried or formed by other means.

Additionally, if the gold islands are partially embedded into glass substrate, this way also can be used for gold islands transfer the other material like described above and then the spikes can be potentially grown at this material.

### Example 3

### Thick Gold and Continuous Gold Films

After the successful formation of spikes on the big islands that have been obtained by evaporation and high temperature annealing of thick gold film (20 nm) the following was carried out: (1) obtain bigger structures that could be formed via evaporation and annealing of thicker gold films (thicker than 20 nm) and establish the peculiarities of the structures formed; (2) grow the spikes on the newly formed structures; (3) grow the spikes on continuous film of gold formed via evaporation only without annealing of thick films (>30 nm).

At the first set of samples, thick gold films (30, 50 and 100 nm) were evaporated on glass and annealed according to the standard procedure similar to the cases of 0.1-20 nm of gold nominal evaporation thickness that have been considered earlier. After the annealing process, the procedure of spikes grow according to protocol 1 was carried out.

For the second set of samples, the same gold films (30, 50 and 100 nm) on glass were used. However, the annealing step was skipped and spikes were directly grown on continuous gold film after evaporation. It should be noted that without annealing the gold films have poor adhesion to the glass surface that may influence on the gold film stability. A solution was to evaporate intermediate adhesive layer (2-3 nm) of chromium or titanium with further conversion onto titanium oxide on glass prior gold evaporation.

According to the obtained SEM images, after annealing of gold film of 30 nm of nominal evaporation thickness, it was observed that the gold islands were formed. Their shape is similar to those ones that have been obtained by annealing of 20 nm gold film. However, the size of the structures even reached a few micrometers and their shape was more complex. Nevertheless, almost all of the structures represent spherical, elliptical, hexagonal, or polygonal crystalline structures with clearly observable facets and flat fringes, similarly to the cases that correspond to 15 or 20 nm of gold film by nominal thickness.

SEM images of the structures obtained after annealing of 50 nm gold film represent a transition state between the formation of continuous film and discontinuous structures and consisting of a combination of extremely big islands (gold “continents”) having size bigger than 10 μm and gold islands of the shape that is similar to those ones obtained after annealing of 30 nm of gold film. On gold continents the sharp flat fringes were clearly observable.

SEM images of the gold structures obtained after evaporation and annealing of 100 nm (nominal evaporation thickness) gold show existence of a continuous film with holes (voids, cavities or wells) in its structure. The film is confirmed to be as continuous by checking its conductivity of 1 Ohm/1 cm point-to-point.

Next, the formation of the spikes on each type of structures was studied. It was found that in case of gold islands formed after evaporation and annealing of 30 nm gold film the spikes uniformly cover all the islands on a slide. It was clearly seen that the edges of the islands have remained naked (lacking presence of spikes) while top and side facets that are expected to have ‘111’ crystallographic orientation are completely and uniformly covered with the spikes, like in case of islands that obtained in after 15 or 20 nm of gold film evaporation and annealing. However, in this case the discussed effects are more pronounced. Spikes geometry is also appeared to be uniform and independent on gold island shape or size: even islands of complex geometry are covered with the spikes in a similar manner compared to the “standard: simple ones having more round geometry. According to prelaminar AFM imaging it was revealed that the spikes possess truncated conical shape with fillet edge having height of 4-8 nm and diameter of the base of 10-20 nm.

SEM images of the islands with spikes grown on the structures after evaporation of 50 nm of gold showed the same tendency of uniform spikes formation on every island across found at the surface. It is clearly shown that the spikes formation occurred even on gold continents in a similar manner like in the case of nano-islands forming a kind of “gold nanoforest”. The entire surface of gold structures was uniformly covered with the spikes while the edges are also still become naked meaning that the top and the side facets of the gold continent has the same crystallographic orientation of ‘111’-type similar to the gold islands formed after evaporation and annealing of 15 or 20 nm by nominal thickness.

According to SEM images the procedure of the spikes growth is applicable to create a continuous large gold film (at least of 22×9 mm) entirely covered with spikes supporting the hypothesis of electroless gold modification. It was shown that they completely and uniformly cover the surface of gold that formed via evaporation and annealing of 100 nm thickness film. It was noticed that there were some spots (ca. 30-50 nm in diameter) that remained uncovered with the spikes because they have different crystallographic orientation (different from ‘111’), but their appearance was prevented by increasing the annealing time. The existence of voids in the structure of the gold film itself may serve now as ‘plasmonic cavities’ for different applications.

Growth of spikes on a continuous gold surface of 30, 50 and 100 nm in thickness without application of annealing step was also successful. The SEM images show that the spikes also uniformly cover the surface of all the films like in case of previous samples when the spikes were grown on annealed structures. However, in this case, due to the gold film lacking thermal pretreatment and hence gold did not well structured in terms of facets orientation, the spikes are located less dense compared to the annealed films. The simultaneous existence of different orientation directions without the prevailing one in all the cases leads to structural similarity of the end-up structures regardless the initial film thickness. All the samples remained conductive after spikes growth like in the case of annealed 100 nm thickness film. To avoid cavities appearing in gold films after annealing is to evaporate more than 100 nm of gold (e.g. 120 nm) to increase the amount of deposited gold on the surface.

According to the results obtained, any surface (flat, curved or having complex geometry obtained by means of any suitable technique) that is covered with gold film (continuous or discontinuous) obtained by means of different deposition ways (evaporation/sputtering/adsorption from solution) and integrated into a surface in such a way that the gold is structured with a specific crystallographic orientations after thermal treatment and/or being as a part of a substrate itself providing that the substrate is quite stable to this thermal treatment procedure, can be a subject of electroless modification with spikes according to methods of this invention. This in turn leads to the demonstration that in a specific surface geometry organization, the potential enhancement effects may be transferred from “surface-enhanced” to “volume-enhanced”. The spikes growth on a continuous gold film also allowed to use a reflectance mode to observe changes that may occur on it. Moreover, it also does not change the fact that any patterns can be prepared using continuous (preferably annealed) gold film for spikes growth in combination with patterning techniques, such as lithography as shown herein above.

1. A nanostructure comprising:
an isolated metallic base;
metallic nano-protrusions comprising a first end and a second end;
wherein, said first end is attached to said base and said second end is exposed to the environment; and
wherein said metallic base and said metallic nano-protrusions comprise a noble metal or an alloy comprising a noble metal.

2. The nanostructure of claim 1, wherein the shape of said nano-protrusions is selected from spikes, rounded structures, rods, balls, domes, squares, rectangles, oval, irregular-shaped or any combination thereof.

3. The nanostructure of claim 1, wherein the length of at least one dimension of said nanostructure ranges between 1 nm and 2 mm.

4. The nanostructure of claim 2, wherein:
said base comprises a flattened shape, wherein the length of at least one lateral dimension of said shape is larger than the height of said shape; or
wherein said base comprises a non-flattened shape, wherein the length of at least one lateral dimension of said shape is smaller or equivalent to the height of said shape.

5. The nanostructure of claim 4, wherein the length of at least one lateral dimension of said base ranges between 1 nm and 100 μm.

6. The nanostructure of claim 4, wherein the height of said base ranges between 1 nm and 1 μm.

7. The nanostructure of claim 1, wherein at least a portion of said base is covered by said nano-protrusions.

8. The nanostructure of claim 7, wherein said base comprises at least one facet.

9. The nanostructure of claim 8, wherein said at least one facet is covered by said nano-protrusions.

10. The nanostructure of claim 8, wherein one portion of said at least one facet is covered by said nano-protrusions and another portion of said facet is not covered by said nano-protrusions.

11. The nanostructure of claim 10, wherein the inner area on the surface of said facet is covered by said nano-protrusions and the outer perimeter area of said facet is not covered by said nano-protrusions.

12. The nanostructure of claim 4, wherein said flattened shape assumes a hexagonal, rectangular, circular, oval, triangular or cylindrical shape.

13. The nanostructure of claim 1, wherein the number of nano-protrusions of each nanostructure ranges between 2 and 1,000,000.

14. The nanostructure of claim 1, wherein the spacing between the nano-protrusions ranges between 1 nm and 100 nm.

15. The nanostructure of claim 1, wherein said noble metal comprises any of the following selected from: Au, Ru, Rh, Pd, Ag, Re, Os, Ir and Pt.

16. The nanostructure of claim 1, wherein said alloy is a gold alloy.

17. (canceled)

18. The nanostructure of claim 1, wherein said alloy is selected from alloys comprising Ru, Rh, Pd, Ag, Re, Os, Ir and Pt.

19. The nanostructure of claim 1, wherein the length of said nano-protrusions ranges between 1 nm and 10 μm.

20. The nanostructure of claim 1, further comprising a substrate wherein said base of said nanostructure is attached to said substrate.

21. The nanostructure of claim 20, wherein said nanostructure is at least partially embedded in said substrate.

22. The nanostructure of claim 1, wherein said nanostructure is crystalline.

23. The nanostructure of claim 22, wherein said nanostructure is polycrystalline or is a single crystal.

24. An array of nanostructures attached to a substrate, said nanostructures comprise the nanostructure of claim 1.

25. The array of claim 24, wherein said substrate comprises silicon dioxide or glass.

26. (canceled)

27. The array of claim 24, wherein the spacing between adjacent nanostructures ranges between 1 nm and 10 μm.

28. A system for performing analysis of an analyte, said system comprising:
an array of nanostructures attached to a substrate, said nanostructures comprise the nanostructure of claim 1, wherein said analyte is bound directly or indirectly to said nanostructure;
a light source;
a light detector;
a processor;
wherein said light source is configured to irradiate said array, said detector is configured to detect optical signals obtained from said array and said processor is configured to process said signal to yield analytical results.

29. The system of claim 28, wherein said analyte comprises an atom, an ion, a molecule, a chemical compound, a biomolecule, a nanoparticle, a biological cell, a component of a biological cell, a toxin, a polymer, a perfluoromolecule or any combination thereof.

30-57. (canceled)
